(19) United States
(12) Patent Application Publication
(10) Pub. No.: US 2021/0356376 A1
(43) Pub. Date: Nov. 18, 2021

(54) FUSED-REFERENCE PARTICLE BASED NORMALISATION FOR IMAGING MASS SPECTROMETRY

(71) Applicants: Fluidigm Canada Inc., Markham (CA); The Governing Council of the University of Toronto, Toronto (CA)

(72) Inventors: Taunia Closson, Markham (CA); Vladimir Baranov, Richmond Hill (CA); Mitchell A. Winnik, Toronto (CA)

(73) Assignees: Fluidigm Canada Inc., Markham, ON (CA); The Governing Council of the University of Toronto, Toronto, ON (CA)

(21) Appl. No.: 17/274,252

(22) PCT Filed: Sep. 9, 2019
(86) PCT No.: PCT/US19/50203

§ 371 Date: Mar. 8, 2021

Related U.S. Application Data
(60) Provisional application No. 62/729,219, filed on Sep. 10, 2018.

(51) Int. Cl. G01N 15/10; G01N 33/68; H01J 49/00

(52) U.S. Cl. CPC G01N 15/1012; G01N 33/6848; G01N 2015/1018; H01J 49/0009; H01J 49/0004

(57) ABSTRACT

This disclosure relates to reagents and their use for elemental imaging mass spectrometry of biological samples.

### CROSS REFERENCE TO RELATED APPLICATION

This PCT application claims priority to U.S. Provisional Patent Application No. 62/729,219, filed Sep. 10, 2018, the entire contents of which are incorporated by reference for all purposes.

### FIELD OF THE INVENTION

The invention relates to an imaging mass calibrator, a method for making the same, a method for monitoring the performance of a mass imaging instrument using the same, and a method of calibrating an instrument using the same.

### BACKGROUND

In imaging mass cytometry, a biological sample is labelled with mass tags. For example, specific binding partners, such as antibodies, may be conjugated to mass tags and used to label specific proteins in a biological sample, such as a cell smear or tissue section. Elemental analysis of the labelling atoms present in the mass tags allows for target species (e.g. proteins and nucleic acids) present in the sample to be identified, and in some cases quantified. As such, quantitation of the mass tags at different locations in a biological sample can provide important insights into its biology, such as tumor oncology. Laser ablation-inductively coupled plasma-mass spectrometry (LA-ICP-MS), amongst other techniques, has been used for imaging biological samples, including those labelled with mass tags in imaging mass cytometry. Imaging mass spectrometry (i.e. without labelling with mass tags) can detect atoms normally present in samples.

Quantitation of elemental ions detected in techniques such as mass spectrometry often requires comparison to a standard of known elemental composition and amount. However, methods of quantitation, or more broadly normalization, by reference to a standard run before or after the sample do not account for instrument sensitivity drift during the imaging of the sample. Depending on the system and application, instrument sensitivity drift can be caused by a number of factors including ion optics drift, surface charging, detector drift (e.g., aging), temperature and gas flows drifts affecting diffusion, and electronics behaviour (e.g., plasma power, ion optics voltages, etc). Furthermore, such drifts in instrument sensitivity could also occur between imaging different samples. Therefore, should an accurate comparison of the signal intensities acquired during imaging of different samples be desired, absolute normalisation of the signal intensities using a quantifiable standard of known elemental composition and amount would be required.

### SUMMARY OF THE INVENTION

The inventors of the present invention have addressed a need for reliable calibrators for imaging mass cytometry and imaging mass spectrometry. The invention therefore provides methods and an apparatus for the normalisation and absolute calibration of the signal intensity detected in a mass imaging apparatus (e.g. imaging mass cytometer, imaging mass spectrometer), enabling precise and absolute quantitation of target species (also termed analytes) in samples. In particular, the inventors have found that fusing reference particles comprising a known elemental composition and known amount of reference atoms to a sample carrier provides an imaging mass calibrator that can be used in the absolute calibration of the signal intensity of a mass imaging apparatus (e.g. imaging mass cytometer, imaging mass spectrometer). In addition, the inventors have found that the calibrator of the invention can be used to monitor the signal intensity detected both during the imaging of a sample and between the imaging of different samples, thus allowing the signal intensity to be normalised to account for any drifts in instrument sensitivity that can occur.

Thus, the invention provides imaging mass calibrators and methods for making the same. The present invention also provides methods for monitoring performance of a mass imaging apparatus (e.g. imaging mass cytometer, imaging mass spectrometer) using the calibrator of the invention, methods for calibrating a mass imaging apparatus (e.g. imaging mass cytometer, imaging mass spectrometer) using the same, and methods for normalizing detector intensity of amass imaging apparatus (e.g. imaging mass cytometer, imaging mass spectrometer) using the same. The present invention further provides methods of imaging samples using the same. The calibrator of the invention may alternatively or in addition be used as a standard in imaging mass cytometry and imaging mass spectrometry.

The invention also provides a method for making an imaging mass calibrator, comprising the steps of contacting a sample carrier with a suspension comprising at least one reference particle wherein the at least one reference particle comprises at least one reference atom, and fusing the at least one reference particle onto the sample carrier.

The invention also provides a method for monitoring the performance of an instrument, the method comprising providing an imaging mass calibrator comprising a sample carrier with at least one reference particle fused thereto, wherein the at least one fused reference particle comprises at least one reference atom, and determining an average integral signal intensity per fused reference particle by sampling and detecting elemental composition and amount.

The invention also provides an imaging mass calibrator with at least one reference particle fused to the sample carrier, and where the at least one reference particle comprises at least one reference atom.

The invention further provides a method for calibrating an mass imaging apparatus (e.g. imaging mass cytometer) comprising the steps of providing a imaging mass calibrator comprising a sample carrier with at least one reference particle fused thereto, wherein the at least one reference particle comprises at least one reference atom, and determining an average integral signal intensity per fused reference particle.

The invention also provides a method of imaging a sample comprising the steps of: (i) providing an imaging mass calibrator comprising a sample carrier with at least one reference particle fused thereto, wherein the at least one fused reference particle comprises at least one reference atom and wherein the sample is on the sample carrier, (ii) contacting the sample with solution comprising at least one mass tag wherein the mass tag comprises at least one labelling atom, (iii) sampling at least one fused reference particle, determining an average integral signal intensity per fused reference particle, and (iv) performing imaging mass cytometry and/or imaging mass spectrometry on the sample to obtain an image.

The invention further provides a method of imaging a sample comprising the steps of: (i) providing a sample on a sample carrier, preparing an imaging mass calibrator wherein the imaging mass calibrator comprises the sample on the sample carrier and wherein the sample carrier has at least one reference particle fused thereto and wherein the at least one fused reference particle comprises at least one reference atom, (ii) contacting the sample with solution comprising at least one mass tag wherein the mass tag comprises at least one labelling atom, (iii) sampling at least one fused reference particle, determining an average integral signal intensity per fused reference particle, and (iv) performing imaging mass cytometry or imaging mass spectrometry on the sample to obtain an image.

### BRIEF DESCRIPTION OF THE FIGURES

FIG. 1. Before and after heating (200° C., 10 minutes) tissue images using MCD Viewer with a minimum threshold of 1 for all three channels: Red=Vimentin, Green=CD45, and Blue=DNA. The maximum signal intensity is detected before and after heating. The data shows that no significant drop in signal detected for the respective mass tags was observed as a resulting of heating the sample carrier.

FIG. 2. Average integral intensities per fused bead for EQ4 beads sampled each hour for approximately 24 hours. Four to seven beads were averaged for each data point with approximately one hour between sampling. Standard deviation across all beads over the 24 hour period are less than 15%.

FIG. 3. Optical microscopy images of EQ4 beads on a sample carrier. The samples carriers were prepared according to the method recited in Example 3. The images show how the method of the invention provides sample carriers with individually localised reference particles, facilitating individual ablation of isolated reference particles and quantitative calibration and normalisation.

FIG. 4. Optical microscopy images of the EQ4 beads fused to a sample carrier after heating for either 10, 20, or 30 minutes.

### DETAILED DESCRIPTION OF THE INVENTION

The present invention relates to calibrators for imaging mass spectrometry and imaging mass cytometry, methods of making them and their applications. As explained below in detail, the inventors have determined that fusing reference particles to sample carriers results in reliable standards that address the shortfalls of other techniques. Such other techniques include spin coating metal atoms in a solution onto a sample carrier. However, this technique suffers from a lack of reproducibility, as the preparation of these standards necessarily results in a variation in the elemental composition and amount of the reference atoms present on the calibrator.

The inventors have determined that using reference particles comprising a consistent amount of a known elemental composition (i.e. reference atoms) offers a solution to these problems and provides imaging mass calibrators that can be used in the absolute calibration and normalisation of signal intensity. Significantly, the inventors discovered that sampling at least one of these particles and detecting an integral signal intensity per particle allows for quantitative calibration of the signal intensity to the amount of reference atoms present in the particle(s). Thus, the imaging mass calibrators of the invention allow for comparison of the signal intensity detected for different samples, in addition to the signal intensity detected for different samples imaged on different cytometers or spectrometers.

The inventors discovered that fusing the particles to the sample carrier facilitated sampling of the entire particle, which would otherwise not be possible due to an interaction of laser radiation with the reference particle, which results in a lateral displacement of the reference particle on the sample carrier and no or only partial detection of the reference atoms present in the reference particle. Thus, by fusing the reference particle with the sample carrier, the inventors have enabled sampling of the whole particle and so the integral signal intensity associated with the entire reference particle can be detected. Sampling the whole particle allows for absolute comparison of the integral signal intensity with the amount of reference atoms known to be in the particle.

Mass cytometry, including imaging mass cytometry, relies on the labelling of target species (also referred to as analytes herein and in the field) on or in a sample using mass-tagged SBPs (an SBP is a member of a specific binding pair) that bind to specific analytes (proteins, nucleic acids, sugars, metabolites etc.). When the analytes are part of a cell, then the SBPs can be applied to label analytes on or in the cell.

Imaging mass spectrometry detects atoms that are naturally present in a sample, e.g. metals in enzymes. The invention enables new ways to analyse analytes naturally present in a sample in a quantitative manner.

### Imaging Mass Calibrator

The present invention provides an imaging mass calibrator. As will be described herein, the imaging mass cytometry calibrator of the invention may be used for calibrating a system, for example an imaging mass cytometer, through the comparison of the signal intensity detected from sampling a known amount of reference atoms present in reference particles. The imaging mass calibrator of the invention can therefore be used to account for the variation in the signal intensity detected that may result from flux in the detector or laser of an imaging mass cytometer. The imaging mass calibrator of the invention can be used as a standard for normalizing the detected signal intensity during and between sampling of samples. In addition, the imaging mass calibrator of the invention can be used to plot a calibration curve for the detected signal intensity and can therefore be used in the absolute quantification of the signal intensity detected from sampling a sample.

The imaging mass cytometry calibrator of the invention comprises a sample carrier with at least one reference particle fused to the sample carrier, wherein the at least one fused reference particle comprises at least one reference atom.

In some embodiments, the sample carrier comprises at least 2, such as at least 3, at least 5, at least 10, at least 50, at least 100, at least 500, at least 1000, at least 2000, at least 5000, such as at least 10000 fused reference particles. The reference particles may all be the same. In some embodiments, the reference particles differ. For instance, the reference particles may differ in their elemental composition (i.e. the reference atoms which they contain), and the amount of reference atom(s) per particle. When different reference particles are used, there will typically be multiple of each type of reference particle. The group of identical reference particles is herein termed a “set”.

The particles may be dispersed on the sample carrier such that substantially all of the particles are located individually (i.e. discretely) on the sample carrier, such that each fused reference particle can be individually identified and sampled. Those skilled in the art will appreciate that the sample carrier may further comprise some fused reference particles that have agglomerated on the sample carrier and thus these agglomerates may be unsuitable for sampling for calibration and normalisation of signal intensity. For example, up to 2%, such as up to 5%, up to 8%, up to 10%, up to 15%, or up to 20% of the fused particles may be agglomerated on the sample carrier of the imaging mass calibrator of the invention. In other words, at least 80% of the particles are individually isolated on the imaging mass calibrator, such as at least 85%, at least 90%, at least 92%, or at least 95%.

In some embodiments, the fused reference particles have a diameter of at least 1 μm, for instance at least 2 μm, at least 3 μm, at least 5 μm, at least 8 μm or at least 10 μm. In some embodiments, the fused reference particles have a diameter less than 30 μm, for instance less than 20 μm, less than 15 μm or less than 10 μm. In some embodiments, the particles have a diameter of between 1 and 20 μm, such as 1 and 15 μm, such as between 1 and 10 μm, such as between 2 and 8 μm,

Imaging mass cytometry typically employs detection of multiple different labelling atoms on different mass channels, which are used to distinguish between the different analytes on the sample that have been labelled with different mass tags. Thus in certain embodiments, the imaging mass calibrator of the invention comprises at least one fused reference particle comprising multiple different reference atoms, for example at least two, at least three, at least four, at least five, at least six, at least seven, at least eight, at least nine, at least 10, at least 20 or at least 50 different reference atoms. By different reference atoms as used herein is meant that each different reference atom has a different atomic mass (e.g. different elements and isotopes therefore). The imaging mass calibrator of the invention can therefore be used in the calibration and normalisation of multiple mass channels, including the mass channels used in the detection of the labelling atoms found in mass tagged SBPs that have labelled a sample. In addition, the imaging mass calibrator can be used to calibrate mass channels used to detect atoms naturally present in un-labelled biological samples.

Those skilled in the art will appreciate that the integral signal associated with sampling a known number of particular reference atoms may be different from that associated with sampling the same number of a different reference atom (for instances because of differences in the behaviours of the reference atoms e.g. ionisation efficiency). The amount of reference atoms present in the fused reference particles can therefore be selected to provide a substantially consistent signal intensity for each reference atom detected for each mass channel that is being normalised/calibrated. That is to say, if the same absolute number of atoms of a first reference atom provides a lower signal at the detector than a second reference atom, then a greater absolute quantity should be provided in the reference particle. Accordingly, the present invention may further provide an imaging mass calibrator comprising at least one fused reference particle comprising multiple different reference atoms, for example at least two, at least three, at least four, at least five, at least six, at least seven, at least eight, at least nine, at least 10, at least 20 or at least 50 different reference atoms, wherein the amount of each reference atom in the at least one fused reference particle may be different from the amount of the other reference atoms present in the reference particle.

In some embodiments, the imaging mass calibrator of the invention may comprise more than one fused reference particles for example the imaging mass calibrator may comprise at least two, at least three, at least four, at least five, at least six, at least seven, at least eight, or at least ten sets of at least one fused reference particle, wherein each set of at least one reference particle individually comprises a different reference atom. Isolating different sets of reference particles to discrete regions of the sample carrier allows for ease of identification of the respective set of reference particles based on their location on the sample carrier. Thus, different sets of particles comprising different specific reference atoms, combination of reference atoms, and/or amount of reference atoms can easily be identified, facilitating plotting of calibration curves for multiple different mass channels of the detector of an imaging mass cytometry. Methods for such calibration, including plotting calibrations curves, will be discussed (see page 48). Thus in certain embodiments, the imaging mass calibrator of the invention comprises more than one fused particle, for example the imaging mass calibrator may comprise at least two, three, four, five, six, seven, eight, or at least ten sets of at least one fused reference particle, wherein each set of at least one fused reference particle is located in a discrete region of the sample carrier. Thus, the invention provides an imaging mass calibrator comprising more than one set of fused reference particles, wherein the user can easily identify a specific set of at least one fused reference particle, wherein said set of at least one fused reference particle comprises a specific reference atom, combination of reference atoms, and/or amount of reference atoms.

In some embodiments, each different set of reference particles is fused to a different region on the sample carrier. The area for each set may be for instance 1 mm×1 mm, 2 mm×2 mm, 4 mm×4 mm, 6 mm×6 mm, 8 mm×8 mm, a 10 mm×10 mm or at 10 mm×20 mm. In some embodiments, each discrete area comprises at least 5, such as at least 10, at least 25, at least 50 or at least 100 reference particles of a set. In some embodiments, the imaging mass calibrator comprises a sample carrier comprising at least two discrete areas with reference particles fused thereto, for example at least 3, at least 4, at least 5, at least 6, at least 8 or at least 10 discrete areas.

As detailed below, quantitation of different labelling atoms requires the plotting of calibration curves for multiple mass channels of the detector being used to detect the labelling atoms. Thus in certain embodiments, the imaging mass calibrator of the invention comprises more than one set of at least one fused reference particle, wherein each set of at least one fused reference particle comprises multiple different reference atoms, wherein each set of at least one fused reference particle comprises a different amount of the multiple different reference atoms. For example, each set of fused reference particle may comprise the same mixture of reference atoms, but at different amounts. The imaging mass calibrator of the invention can therefore be used to plot calibration curves for an imaging mass cytometry detector for multiple mass channels.

The imaging mass calibrator of the invention may further comprise a sample, wherein the sample has been labelled with one or more mass tags. Thus the invention provides a labelled sample on an imaging mass calibrator, wherein the sample has been labelled with at least one labelling atom in at least one mass tag (e.g. at least 2, at least 5, at least 10 or at least 20 labelling atoms), wherein the imaging mass calibrator comprises at least one reference particle, fused to the sample carrier (e.g. at least 2, at least 5, at least 10 or at least 20 reference atoms), that comprises a reference atom that is the same as the labelling atom(s).

### Reference Particles

The reference particles fused to the sample carrier in the present invention comprise a known elemental composition and amount of reference atoms, to allow quantitative normalisation of detected signal intensity during imaging and absolute calibration of the detector of an imaging mass cytometer. Accordingly, the reference particle for use in the invention may comprise a variety a forms provided that the above condition is satisfied, i.e. that the reference particle comprises a known elemental composition and amount of reference atoms, and that the particle is capable of being fused to a sample carrier. As discussed below in more detail, the particle may be capable of being fused to the sample carrier by hearing. In some embodiments, the particle is capable of being fused to the sample carrier by solvent annealing. There are several methods known in the art by which a known elemental composition and amount of reference atoms can be incorporated into a reference particle. Accordingly, the present invention provides the use of at least one reference particle in a method of making an imaging mass calibrator. The invention also provides suspensions of beads (i.e. beads in a solvent), wherein the concentration of beads in the suspension is sufficiently high that they can be used to make an imaging mass calibrator.

Those skilled in the art will appreciate that the reference particles will be of a certain size to allow both the incorporation of a sufficient amount of reference atoms to ensure adequate signal detection, however the particles should not be of such a size that the time required to ablate the whole particle from the sample carrier becomes impractical. Thus, those skilled in the art will appreciate that before fusing the reference particles for use in the invention may have a diameter in the of 1 μm to 50 μm, including 1 μm to 40 μm, 1 μm to 30 μm, 1 μm to 20 μm, or 1 μm to 10 μm, or 1 μm to 5 μm. In some embodiments the particles are about 3 μm in diameter. The reference particles of the present invention comprise at least one reference atom (for discussion of the types of reference atom see page 15). In some embodiments, the reference particle comprises at least 10,000, such as at least 50,000, at least 100,000, at least 500,000, at least 1,000,000, at least 5,000,000, at least 10,000,000, at least 30,000,000, at least 50,000,000, at least 100,000,000, at least 200,000,000, at least 500,000,000, at least 1,000,000,000, at least 5,000,000,000, or at least 10,000,000,000 reference atoms. In some embodiments, the particles discussed in this paragraph have an average diameter of less than 10 μm.

The reference particles for use in the invention may comprise one type of reference atom. In some embodiments, the reference particles comprise more than one different reference atom, for example at least 2, at least 3, at least 4, at least 5, at least 6, at least 8, at least 10, at least 20 or at least 30 different reference atoms. In other words, the reference particles of the present invention may comprise mixtures of different reference atoms. In some embodiments the reference particles of the invention comprise between at least 10,000, such as at least 50,000, at least 100,000, at least 500,000, at least 1,000,000, at least 5,000,000, at least 10,000,000, at least 30,000,000, at least 50,000,000, at least 100,000,000, at least 200,000,000, at least 500,000,000, at least 1,000,000,000, at least 5,000,000,000, or at least 10,000,000,000 of each type of reference atom. In some embodiments, the particles discussed in this paragraph have an average diameter of less than 10 μm.

As discussed, the integral signal associated with sampling a particular reference atom may be different from that associated with sampling a different reference atom for the same absolute quantity of reference atoms. Therefore, the particles for use in the invention may therefore comprise a mixture of different reference atoms wherein the amount of the different reference atoms present in the fused reference particles is different and is selected to provide a substantially consistent signal intensity for each reference atom detected by each mass channel being normalised/calibrated.

In some embodiments of the invention, different sets of reference particles comprise the same mixture of different reference atoms, however each set of reference particles comprises a different amount of the different reference atoms. Thus, the particles for use in the invention can be used to make an imaging mass calibrator that can be used to plot calibration curves for multiple mass channels. E.g., the standard curve can be generated by a series, for instance a series of 2-fold differences in the number of reference atoms, a series of 3-fold differences in the number of reference atoms, a series of 5-fold differences in the number of reference atoms or a series of 10-fold differences in the number of reference atoms.

In some embodiments, a set of reference particles comprises n reference atoms of each type, where n=10,000,000-30,000,000. Sets of reference particles may comprise at least (n×2), at least (n×4), at least (n×8), at least (n×16), or at least (n×32). In some embodiments, sets of reference particles comprise at least (n×3), such as at least (n×9), at least (n×27), or at least (n×81). In some embodiments, sets of reference particles comprise at least (n/32), such as at least (n/16), at least (n/8), at least (n/4), or at least (n/2) of each type of reference atom. In some embodiments, sets of reference particles comprise at least (n/81), such as at least (n/27), at least (n/9), or at least (n/3). In some embodiments, the particles discussed in this paragraph have an average diameter of less than 10 μm.

In some embodiments, a set of reference particles comprises n reference atoms of each type, where n=10,000,000-30,000,000. Sets of reference particles may comprise at least (n×10²) of each type of reference atom, such as at least (n×10³), at least (n×10⁴), at least (n×10⁵), at least (n×10⁶), at least (n×10⁷) of each type of reference atom. In some embodiments, a set of reference particles may comprise at least (n×10⁻⁵), at least (n×10⁻⁴), at least (n×10⁻³), or at least (n×10⁻²) of each type of reference atom. In some embodiments, the particles discussed in this paragraph have an average diameter of less than 10 μm.

In some embodiments, a set of reference particles comprises n×10⁻⁵-n×10⁵ of each type of reference atom, such as n×10⁻⁴-n×10⁵ of each type of reference atom, n×10⁻³-n×10³ of each type of reference atom, n×10⁻²-n×10² of each type of reference atom, or n×10⁻¹-n×10¹ of each type of reference atom; where n=10,000,000-30,000,000.

In some embodiments, a set of reference particles comprises n/m, n and n×m of each type of reference atom; where n=10,000,000-30,000,000, where m=3, 4, 5, 6, 7, 8 or 9 or 20. In some embodiments, a set of reference particles comprises one or more of sets comprising n/m⁵ of each type of reference atom, n/m⁴ of each type of reference atom, n/m³ of each type of reference atom, n/m² of each type of reference atom, n×m² of each type of reference atom, n×m³ of each type of reference atom, n×m⁴ of each type of reference atom, n×m⁵ of each type of reference atom and n×m⁶ of each type of reference atom; where n=10,000,000-30,000,000, and wherein m=3, 4, 5, 6, 7, 8 or 9 or 20.

In some embodiments, the reference particles for use in the invention comprise at least five different types of reference atom, for example ¹⁴⁰Ce, ¹⁵¹Eu, ¹⁵³Eu, ¹⁶⁵Ho, ¹⁷⁵Lu, and each reference particle comprises at least 1000, such as at least 5000, at least 10,000, at least 250,000, at least 500,000, at least 1,000,000, at least 2,500,000, at least 5,000,000, at least 10,000,000, at least 100,000,000, at least, 200,000,000 or at least 300,000,000 reference atoms. In some embodiments the reference particles of the invention comprise at least five different types of reference atom, for example ¹⁴⁰Ce, ¹⁵¹Eu, ¹⁵³Eu, ¹⁶⁵Ho, ¹⁷⁵Lu, and each reference particle comprises between 1,000-300,000,000, 2,000-200,000,000, 5,000-175,000,000, 50,000-150,000,000, 100,000-125,000,000, 200,000-110,000,000, 1,000,000-100,000,000, 10,000,000-95,000,000, 30,000,000-90,000,000, 40,000,000-80,000,000, or 50,000,000-70,000,000 reference atoms in total. In some embodiments, the particles discussed in this paragraph have an average diameter of less than 10 μm.

In some embodiments, the reference particles for use in the invention have a diameter between 1 μm to 50 μm and between n×10⁻⁵-n×10⁷ reference atoms in total, such as a diameter between 1 μm to 40 μm and between n×10⁻⁵-n×10⁶ reference atoms in total, a dimeter between 1 μm to 30 μm and between n×10⁻⁵-n×10⁵ reference atoms in total, a dimeter between 1 μm to 20 μm and between n×10⁻⁵-n×10⁴ reference atoms in total, a diameter between 1 μm to 10 μm and between n×10⁻⁴-n×10³ reference atoms in total, or a diameter between 1 μm to 5 μm and between n×10⁻³-n×10² reference atoms in total; where n=10,000,000-30,000,000.

In some embodiments, the reference particles for use in the invention comprise at least five different types of reference atom, for example ¹⁴⁰Ce, ¹⁵¹Eu, ¹⁵³Eu, ¹⁶⁵Ho, ¹⁷⁵Lu, and have a diameter of 1 μm to 50 μm and between 1,000-300,000,000 reference atoms in total, such as a diameter of 1 μm to 40 μm and between 2,000-200,000,000 reference atoms in total, a dimeter of 1 μm to 30 μm and between 100,000-125,000,000 reference atoms in total, a dimeter of 1 μm to 20 μm and between 1,000,000-100,000,000 reference atoms in total, a diameter of 1 μm to 10 μm and between 30,000,000-90,000,000 reference atoms in total, or a diameter of 1 μm to 5 μm and between 50,000,000-70,000,000 reference atoms in total. In some embodiments the particles are about 3 μm in diameter and comprise about 60,000,000 reference atoms in total.

In some embodiments, the reference particles for use in the invention comprise at least five different types of reference atom, for example ¹⁴⁰Ce, ¹⁵¹Eu, ¹⁵³Eu, ¹⁶⁵Ho, ¹⁷⁵Lu, and have a diameter of 1 μm to 50 μm and between 1,000-100,000,000 of each type of reference atom, for example a diameter of 1 μm to 40 μm and between 5,000-50,000,000 of each type of reference atom, a dimeter of 1 μm to 30 μm and between 100,000-30,000,000 of each type of reference atom, a dimeter of 1 μm to 20 μm and between 200,000-20,000,000 of each type of reference atom, a diameter of 1 μm to 10 μm and between 1,000,000-20,000,000 of each type of reference atom, or a diameter of 1 μm to 5 μm and between 10,000,000-20,000,000 of each type of reference atom. In some embodiments the particles are about 3 μm in diameter and comprise about 15,000,000 of each type of reference atom.

Sets of reference particles comprising different amounts of reference atoms can therefore be provided in combination to afford a “calibration series” of reference particles. A calibration series can comprise at least 2 sets, such as at least 3, at least 4, at least 5, at least 6, at least 7, at least 8, at least 9, or 10 or more sets of reference particles comprising different amounts of reference atoms.

In some embodiments, a calibration series comprises at least three sets of reference particles, having n/m, n and n×m of each type of reference atom; where n=10,000,000-30,000,000, where m=4, 5, 6, 7, 8 or 9 or 20. In some embodiments, the calibration series further comprises one or more of sets comprising n/m⁵ of each type of reference atom, n/m⁴ of each type of reference atom, n/m³ of each type of reference atom, n/m² of each type of reference atom, n×m² of each type of reference atom, n×m³ of each type of reference atom, n×m⁴ of each type of reference atom, n×m⁵ of each type of reference atom and n×m⁶ of each type of reference atom; where n=10,000,000-30,000,000, and wherein m equals the value of m in the n/m, n and n×m series.

In some embodiments, the calibration series comprises sets of reference particles comprising at least five different types of reference atom, for example ¹⁴⁰Ce, ¹⁵¹Eu, ¹⁵³Eu, ¹⁶⁵Ho, ¹⁷⁵Lu, and comprise between 1,000,000-3,000,000 reference atoms, 3,000,000-5,000,000 reference atoms, 5,000,000-10,000,000 reference atoms, 10,000,000-20,000,000 reference atoms, 20,000,000-40,000,000 reference atoms, 40,000,000-60,000,000 reference atoms, 60,000,000-80,000,000 reference atoms, 80,000,000-100,000,000 reference atoms, 100,000,000-140,000,000 reference atoms, and/or 140,000,000-200,000,000 reference atoms in total. For example, a calibration series may comprise sets of reference particles comprising about 2,000,000 reference atoms, about 4,000,000 reference atoms, about 7,500,000 reference atoms, about 15,000,000 reference atoms, about 30,000,000 reference atoms, about 50,000,000 reference atoms, about 70,000,000 reference atoms, about 90,000,000 reference atoms, about 120,000,000 reference atoms, and/or about 160,000,000 reference atoms in total. In some embodiments, the particles discussed in this paragraph have an average diameter of less than 10 μm.

In some embodiments, the calibration series comprises sets of reference particles comprising at least five different types of reference atom, for example ¹⁴⁰Ce, ¹⁵¹Eu, ¹⁵³Eu, ¹⁶⁵Ho, ¹⁷⁵Lu, and comprise between 250,000-750,000, 750,000-1,250,000, 1,250,000-3,000,000, 3,000,000-5,000,000, 5,000,000-9,000,000, 9,000,000-14,000,000, 14,000,000-18,000,000, 18,000,000-22,000,000, 22,000,000-34,000,000, and/or 34,000,000-44,000,000 of each reference atom. For example, a calibration series may comprise sets of reference particles comprising about 500,000, about 1,000,000, about 2,000,000, about 4,000,000, about 7,000,000, about 12,000,000, about 16,000,000, about 20,000,000, about 28,000,000, and/or about 39,000,000 of each reference atom. In some embodiments, the particles discussed in this paragraph have an average diameter of less than 10 μm.

In some embodiments, the calibration series comprises sets of reference particles comprising at least five different types of reference atom, for example ¹⁴⁰Ce, ¹⁵¹Eu, ¹⁵³Eu, ¹⁶⁵Ho, ¹⁷⁵Lu, and comprise between 250,000-750,000, 750,000-1,250,000, 1,250,000-3,000,000, 3,000,000-5,000,000, 5,000,000-9,000,000, 9,000,000-14,000,000, 14,000,000-18,000,000, 18,000,000-22,000,000, 22,000,000-34,000,000, and/or 34,000,000-44,000,000 of each reference atom. For example, a calibration series may comprise sets of reference particles comprising about 500,000, about 1,000,000, about 2,000,000, about 4,000,000, about 7,000,000, about 12,000,000, about 16,000,000, about 20,000,000, about 28,000,000, and/or about 39,000,000 of each reference atom.

### Composition of Reference Atoms in the Reference Particles

Different sets of reference particles may comprise different reference atoms/isotopes, different combinations of reference atoms/isotopes, different amounts of the same reference atoms/isotopes, and even different ratios of different reference atoms/isotopes. Accordingly, in some embodiments, all reference atoms in a set of reference particles are of the same atomic mass. Alternatively, a set of reference particles can comprise reference atoms of differing atomic mass but comprise the same amount of each different reference atom. Accordingly, in some instances, a set of reference particles may be formed from reference particles each of which comprises just a single type of reference atom.

In addition, in some instances, a set of reference particles can comprise reference atoms of the same atomic mass and the same amount of said reference atom in each reference particle. Alternatively, in some instances, a set of reference particles may be formed from reference particles each of which comprises more than one different reference atom, for example at least two, at least three, at least four, at least five, at least six, at least seven, at least eight, or at least ten different reference atoms. Sets of reference particles find particular application in the methods detailed herein. In some instances, a set of reference particles may be formed from reference particles comprising different amounts of the same reference atom.

As discussed, the integral signal associated with sampling a particular reference atom may be different from that associated with sampling a different reference atom. Therefore, the particles for use in the invention may therefore comprise a mixture of different reference atoms wherein the amount of the different reference atoms present in the fused reference particles is different and is selected to provide a substantially consistent signal intensity for each reference atom detected by each mass channel being normalised/calibrated. In some embodiments, the reference particles for use in the invention comprise 15,000,000-20,000,000 ¹⁴⁰Ce reference atoms, for example 17,500,000-22,500,000, or 19,000,000-21,000,000 ¹⁴⁰Ce reference atoms; 6,000,000-16,000,000 ¹⁵¹Eu reference atoms, for example 8,500,000-13,500,000, 10,000,000-12,000,000, or about 11,000,000 ¹⁵¹Eu reference atoms; 8,000,000-17,000,000 ¹⁵³Eu reference atoms, for example 9,500,000-14,500,000, 11,000,000-13,000,000, or about 12,000,000 ¹⁵³Eu reference atoms; 2,000,000-12,000,000 ¹⁶⁵Ho reference atoms, for example 4,500,000-9,500,000, 6,000,000-8,000,000, or about 7,000,000 ¹⁶⁵Ho reference atoms; 5,000,000-15,000,000 ¹⁷⁵Lu reference atoms, for example 7,500,000-12,500,000 ¹⁷⁵Lu reference atoms, 9,000,000-11,000,000, or about 10,000,000 ¹⁷⁵Lu reference atoms.

Accordingly, in some embodiments of the invention, different sets of reference particles comprise the same mixture of different reference atoms, however each set of reference particles comprises a different amount of the different reference atoms. Thus, the particles can be used to make an imaging mass calibrator that can be plot calibration curves for multiple mass channels. Sets of reference particles comprising different amounts of reference atoms can therefore be provided in combination to afford a “calibration series” of reference particles. In some embodiments, a calibration series comprises at least 2 sets, such as at least 3, at least 4, at least 5, at least 6, at least 7, at least 8, at least 9, or 10 or more sets of reference particles comprising different amounts of reference atoms.

In some embodiments, a calibration series comprises at least three sets of reference particles, having n×10⁻¹, n and n×10⁻² of each type of reference atom; where n=10,000,000-30,000,000. In some embodiments, the calibration series further comprises one or more of sets comprising n×10⁻⁵-n×10⁻⁴ of each type of reference atom, n×10⁻⁴-n×10⁻³ of each type of reference atom, n×10⁻³-n×10⁻² of each type of reference atom, n×10⁻²-n×10⁻¹, n×10²-n×10³ of each type of reference atom, n×10³-n×10⁴ of each type of reference atom, n×10⁴-n×10⁵ of each type of reference atom, and n×10⁵-n×10⁶ of each type of reference atom; where n=10,000,000-30,000,000.

In some embodiments, a calibration series comprises at least three sets of reference particles, having n/2, n and n×2 of each type of reference atom; where n=10,000,000-30,000,000. In some embodiments, the calibration series further comprises one or more of sets comprising n/64 of each type of reference atom, n/32 of each type of reference atom, n/16 of each type of reference atom, n/8 of each type of reference atom, n/4 of each type of reference atom, n×4 of each type of reference atom, n×8 of each type of reference atom, n×16 of each type of reference atom, n×32 of each type of reference atom and n×64 of each type of reference atom; where n=10,000,000-30,000,000.

In some embodiments, a calibration series comprises sets of reference particles having between 300,000-1,000,000 ¹⁴⁰Ce reference atoms, 1,000,000-1,500,000 ¹⁴⁰Ce reference atoms, 1,500,000-3,500,000 ¹⁴⁰Ce reference atoms, 3,500,000-7,000,000 ¹⁴⁰Ce reference atoms, 7,000,000-11,000,000 ¹⁴⁰Ce reference atoms, 11,000,000-19,000,000 ¹⁴⁰Ce reference atoms, 19,000,000-23,000,000 ¹⁴⁰Ce reference atoms, 23,000,000-30,000,000 ¹⁴⁰Ce reference atoms, 30,000,000-44,000,000 ¹⁴⁰Ce reference atoms, and/or 44,000,000-56,000,000 ¹⁴⁰Ce reference atoms. In some embodiments a calibration series comprises sets of reference particles having about 700,000, about 1,300,000, about 2,500,000, about 5,500,000, about 9,000,000, about 16,000,000, about 21,000,000, about 26,000,000, about 37,000,000, and/or about 51,000,000 ¹⁴⁰Ce reference atoms.

In some embodiments, a calibration series comprises sets of reference particles having between 200,000-500,000 ¹⁵¹Eu reference atoms, 500,000-1,000,000 ¹⁵¹Eu reference atoms, 1,000,000-2,000,000 ¹⁵¹Eu reference atoms, 2,000,000-4,000,000 ¹⁵¹Eu reference atoms, 4,000,000-6,000,000 ¹⁵¹Eu reference atoms, 6,000,000-11,000,000 ¹⁵¹Eu reference atoms, 11,000,000-13,000,000 ¹⁵¹Eu reference atoms, 13,000,000-18,000,000 ¹⁵¹Eu reference atoms, 18,000,000-23,000,000 ¹⁵¹Eu reference atoms, and/or 23,000,000-37,000,000 ¹⁵¹Eu reference atoms. In some embodiments a calibration series comprises sets of reference particles having about 400,000, about 750,000, about 1,500,000, about 3,000,000, about 5,000,000, about 9,000,000, about 12,000,000, about 15,000,000, about 21,000,000, and/or about 29,000,000 ¹⁵¹Eu reference atoms.

In some embodiments, a calibration series comprises sets of reference particles having between 200,000-600,000 ¹⁵³Eu reference atoms, 600,000-1,000,000 ¹⁵³Eu reference atoms, 1,000,000-2,000,000 ¹⁵³Eu reference atoms, 2,000,000-4,000,000 ¹⁵³Eu reference atoms, 4,000,000-7,000,000 ¹⁵³Eu reference atoms, 7,000,000-11,000,000 ¹⁵³Eu reference atoms, 11,000,000-14,000,000 ¹⁵³Eu reference atoms, 14,000,000-18,000,000 ¹⁵³Eu reference atoms, 18,000,000-26,000,000 ¹⁵³Eu reference atoms, and/or 26,000,000-36,000,000 ¹⁵³Eu reference atoms. In some embodiments, a calibration series comprises sets of reference particles having about 400,000, about 800,000, about 1,600,000, about 3,000,000, about 5,500,000, about 10,000,000, about 13,000,000, about 16,000,000, about 22,000,000, and/or about 31,000,000 ¹⁵³Eu reference atoms.

In some embodiments, a calibration series comprises sets of reference particles having between 200-000-300,000, ¹⁶⁵Ho reference atoms, 300-000-700,000 ¹⁶⁵Ho reference atoms, 700-000-1,300,000 ¹⁶⁵Ho reference atoms, 1,300,000-2,800,000 ¹⁶⁵Ho reference atoms, 2,800,000-3,500,000 ¹⁶⁵Ho reference atoms, 3,500,000-7,500,000 ¹⁶⁵Ho reference atoms, 7,500,000-9,000,000 ¹⁶⁵Ho reference atoms, 9,000,000-11,000,000 ¹⁶⁵Ho reference atoms, 11,000,000-17,000,000 ¹⁶⁵Ho reference atoms, and/or 17,000,000-23,000,000 ¹⁶⁵Ho reference atoms. In some embodiments, a calibration series comprises sets of reference particles having about 250,000, about 500,000, about 1,000,000, about 2,000,000, about 3,500,000, about 6,000,000, about 8,000,000, about 10,000,000, about 14,000,000, and/or about 20,000,000 ¹⁶⁵Ho reference atoms.

In some embodiments, a calibration series comprises sets of reference particles having between 200,000-400,000 ¹⁷⁵Lu reference atoms; 400,000-1,000,000 ¹⁷⁵Lu reference atoms; 1,000,000-1,500,000 ¹⁷⁵Lu reference atoms; 1,500,000-3,500,000 ¹⁷⁵Lu reference atoms; 3,500,000-5,500,000 ¹⁷⁵Lu reference atoms; 5,500,000-9,000,000 ¹⁷⁵Lu reference atoms; 9,000,000-11,000,000 ¹⁷⁵Lu reference atoms; 11,000,000-15,000,000 ¹⁷⁵Lu reference atoms; 15,000,000-21,000,000 ¹⁷⁵Lu reference atoms; and/or 21,000,000-31,000,000 ¹⁷⁵Lu reference atoms. In some embodiments, a calibration series comprises sets of reference particles having about 300,000, about 700,000, about 1,300,000, about 2,500,000, about 4,500,000, about 8,000,000, about 10,500,000, about 13,000,000, about 19,000,000, and/or about 26,000,000 ¹⁷⁵Lu reference atoms.

One or more elements or isotopes in the reference particles for use in the invention may be of identical mass to target elements in the sample. In other embodiments, none of the elements or isotopes in the reference particles for use in the invention have masses identical to any of the target elements. Alternatively or in addition to, certain elements or isotopes in the reference particles for use in the invention may be of higher mass than some target elements, or lower mass than other target elements. In certain embodiments, one or more elements or isotopes in the reference particles for use in the invention may be of different mass from any of the target elements. In some instances, as well as the reference atoms, the reference particle may also comprise one or more coding atoms. The coding atom is an atom unique to a set of particles. When the coding atom is detected, it is therefore indicative that a particle from a specific set is being sampled. For instance, this finds particular utility in sets of particles which together form a calibration curve, because in such a series the atomic composition remains the same, merely the amount of each reference atom in the particle difference. Accordingly each point in the curve can be coded with a specific coding atom (or combination thereof forming a barcode), thereby identifying what quantity of the reference atoms that particle contains.

In certain embodiments, the reference particles for use in the invention comprise a fluorescence moiety specific to the amount and identity of the reference atom(s) present in the reference particle. Accordingly, in certain embodiments, the identity and amount of the reference atom(s) present in the reference particle can be identified through fluorescence spectroscopy. Fluorescent moieties which may be employed include Alexa Fluor 350, Alexa Fluor 647, Oregon Green, Alexa Fluor 405, Alexa Fluor 680, Fluorescein (FITC), Alexa Fluor 488, Alexa Fluor 750, Cy3, Alexa Fluor 532, Pacific Blue, Pacific Orange, Alexa Fluor 546, Coumarin, Tetramethylrhodamine (TRITC), Alexa Fluor 555, BODIPY FL, Texas Red, Alexa Fluor 568, Pacific Green, Cy5, and Alexa Fluor 594. the method further comprising the steps of, before wherein the fluorescent tag identifies the reference particle, for example wherein fluorescence microscopy is used to identify the specific set of at least one reference particle.

### Types of Reference Atom

Reference atoms that can be incorporated into the reference particles include any species that are detectable by MS or OES. The reference atoms can be atoms chosen as labelling atoms in line with the present disclosure. However, reference atoms may include those atoms which would not work as labelling atoms. As noted below, typically labelling atoms are chosen based on their absence or very low level presence in the biological sample being analysed. As such, detection of their signal in a labelled sample indicates the presence of the target of a mass-tagged SBP. In some instances, however, the reference atoms include those which are naturally present in the sample. This thus enables quantification of e.g. metals in the active sites of enzymes, or other co-ordinated metals, such as iron in haem or magnesium in chlorophyll, inter alia.

Often the reference atom is a metal. In preferred embodiments, however, the reference atoms are transition metals, such as the rare earth metals (the 15 lanthanides, plus scandium and yttrium). These 17 elements (which can be distinguished by OES and MS) provide many different isotopes which can be easily distinguished (by MS). A wide variety of these elements are available in the form of enriched isotopes e.g. samarium has 6 stable isotopes, and neodymium has 7 stable isotopes, all of which are available in enriched form. The 15 lanthanide elements provide at least 37 isotopes that have non-redundantly unique masses. Examples of elements that are suitable for use as reference atoms include Lanthanum (La), Cerium (Ce), Praseodymium (Pr), Neodymium (Nd), Promethium (Pm), Samarium (Sm), Europium (Eu), Gadolinium, (Gd), Terbium (Tb), Dysprosium (Dy), Holmium (Ho), Erbium (Er), Thulium (Tm), Ytterbium (Yb), Lutetium (Lu), Scandium (Sc), and Yttrium (Y). In addition to rare earth metals, other metal atoms are suitable for detection e.g. gold (Au), platinum (Pt), iridium (Ir), rhodium (Rh), bismuth (Bi), etc. The use of radioactive isotopes is not preferred as they are less convenient to handle and are unstable e.g. Pm is not a preferred reference atom among the lanthanides.

Accordingly, in some embodiments, the reference particles comprise reference atoms corresponding to the labelling atoms present in the mass tags used to label the sample being imaged. Thus allowing the mass channels used during imaging mass cytometry to be normalised and calibrated. However, in some embodiments the reference particles comprise reference atoms corresponding to metals that are naturally occurring in the sample. In these embodiments, the imaging mass calibrator of the invention can be used to calibrate and normalise the signal intensity associated with the mass channels used in imaging mass spectrometry of metal atoms/ions native to the sample, for example when the sample is a biological sample, the reference particles may comprise Selenium (Se), Cobalt (Co), Iron (Fe), Copper (Cu), Nickel (Ni), Arsenic (As), Vanadium (V), Manganese (Mn), Chromium (Cr), Zinc (Zn), and Molybdenum (Mo).

Various numbers of reference atoms can be incorporated into a reference particle depending upon the reference particle used. Greater sensitivity can be achieved when more reference atoms are incorporated into a reference particle. For example, in some embodiments a reference particle comprises at least 1000, such as at least 5000, at least 10,000, at least 250,000, at least 500,000, at least 1,000,000, at least 2,500,000, at least 5,000,000, or at least 10,000,000 reference atoms.

In some embodiments, a reference particle comprises at least five different types of reference atom, for example ¹⁴⁰Ce, ¹⁵¹Eu, ¹⁵³Eu, ¹⁶⁵Ho, ¹⁷⁵Lu, and comprises between 1,000-100,000,000, 5,000-50,000,000, 50,000-40,000,000, 100,000-30,000,000 200,000-20,000,000, 1,000,000-20,000,000, 10,000,000-20,000,000 or 12,000,000-18,000,000 of each type of reference atom. In some embodiments the reference particles of the invention comprise between 1,000-300,000,000, 2,000-200,000,000, 5,000-175,000,000, 50,000-150,000,000, 100,000-125,000,000, 200,000-110,000,000, 1,000,000-100,000,000, 10,000,000-95,000,000, 30,000,000-90,000,000, 40,000,000-80,000,000, or 50,000,000-70,000,000 reference atoms in total. For instance, each different type of reference atom may be present in a copy number of 1,000-100,000,000, 5,000-50,000,000, 50,000-40,000,000, 100,000-30,000,000 200,000-20,000,000, 1,000,000-20,000,000, 5,000,000-20,000,000 or 8,000,000-18,000,000 of each reference atom per particle. As noted below, polymers with narrow molecular weight distributions containing multiple monomer units may be used, each containing a chelator such as diethylenetriaminepentaacetic acid (DTPA) or DOTA.

### Metal Doped Beads

One such means of incorporating reference atoms in known elemental compositions and amounts is via the use of doped beads. Accordingly, reference particles for use in the invention may be metal-doped beads, such as metal-doped polymer beads, for example metal-doped polystyrene beads, such as EQ4 or DM7 beads available from Fluidigm Canada, Inc.

The polymer bead may be doped so as to contain one or more different reference atoms (reference atoms are discussed in more detail below herein, at page 15). Methods of making the doped beads for use in the invention include employing chelated lanthanide (or other metal) ions in miniemulsion polymerizations to create polymer reference particles with the chelated lanthanide ions embedded in the polymer. The chelating groups are chosen, as is known to those skilled in the art, in such a way that the metal chelate will have negligible solubility in water but reasonable solubility in the monomer for miniemulsion polymerization. Typical monomers that one can employ are styrene, methylstyrene, various acrylates and methacrylates, among others as is known to those skilled in the art. For mechanical robustness, the metal-tagged reference particles have a glass transition temperature (T_{g}) above room temperature. Winnik, M. A., et. al., J. Am. Chem. Soc., 2009, 131, 15276 discloses such a dispersion polymerisation methodology for the preparation of doped polymer beads.

In addition, metal doped beads for use in the invention can be prepared by Pickering emulsion polymerisation, where solid particles are added to an emulsion to stabilise the emulsion and prevent coalescence of the dispersed phase. Similarly to mini-emulsion polymerisation, oil-soluble metal chelates can be introduced into Pickering emulsion polymerisations, such that the metal chelate is incorporated into the dispersed monomer phase and thus into the polymer bead upon polymerisation. The particles introduced to stabilise the dispersed phase may be metal-containing nanoparticles, such that the particles formed during the polymerisation reaction further comprise metal-containing nanoparticles.

The polymer bead can be made from a polymer selected from the group consisting of linear polymers, copolymers, branched polymers, graft copolymers, block polymers, star polymers, and hyperbranched polymers. The backbone of the polymer can derived from polyolefins, polymethacrylates, polyacrylates, polymethacrylamides, polyN-alkyl acrylamides, polyN,N-dialkyl acrylamides, polyN-aryl acrylamides, polyN-alkyl methacrylamides, polyN,N-dialkyl methacrylamides, polyN-aryl methacrylamides, polymethacrylate esters, polyacrylate esters and functional equivalents thereof. Polymers beads for use in the invention may also be made from poly(vinylidene fluoride), poly(tetrafluoroethylene), polylactic acid, poly(methylmethacrylate), polystyrene, poly(vinylpyridine), combinations thereof and the like. The polymer can be substituted. In certain embodiments where the polymer bead is a polystyrene bead, the bead is made from a polystyrene homopolymer, or a copolymer comprising monomer units derived from polystyrene, for example a random or block copolymer.

### Metal-Doped Core-Shell Polymer Reference Particles

In some instances, core-shell reference particles are used, in which the metal-doped reference particles prepared by miniemulsion polymerization are used as seed reference particles for a seeded emulsion polymerization to control the nature of the surface functionality. Surface functionality can be introduced through the choice of appropriate monomers for this second-stage polymerization. Additionally, acrylate polymers are advantageous over polystyrene reference particles because the ester groups can bind to or stabilize the unsatisfied ligand sites on the lanthanide complexes. An exemplary method for making such doped beads is: (a) combining at least one reference atom-containing complex in a solvent mixture comprising at least one organic monomer (such as styrene and/or methyl methacrylate) in which the at least one reference atom-containing complex is soluble and at least one different solvent in which said organic monomer and said at least one reference atom-containing complex are less soluble, (b) emulsifying the mixture of step (a) for a period of time sufficient to provide a uniform emulsion; (c) initiating polymerization and continuing reaction until a substantial portion of monomer is converted to polymer; and (d) incubating the product of step (c) for a period of time sufficient to obtain a latex suspension of polymeric reference particles with the at least one reference atom-containing complex incorporated in or on the reference particles therein, wherein said at least one reference atom-containing complex is selected such that upon interrogation of the polymeric reference particle, a distinct mass signal is obtained from said at least one reference atom. By the use of two or more complexes comprising different reference atoms, doped beads can be made comprising two or more different reference atoms. Furthermore, controlling the ratio of the complexes comprising different reference atoms, allows the production of doped beads with different ratios of the reference atoms. In core-shell beads, this may be achieved by incorporating a first reference atom-containing complex into the core, and a second reference atom-containing complex into the shell.

### Polymer-Coated Metal Nanoparticles

Another means of making the reference particles for use in the present invention is to generate particles, such as nanoparticles of metal which have been coated in a polymer. Here, the metal is sequestered and shielded from the environment by the polymer, and does not react when the polymer shell is fused to the sample carrier by heating.

Grafting-to and grafting-from are the two principle mechanism for generating polymer brushes around a nanoparticle. In grafting to, the polymers are synthesised separately, and so synthesis is not constrained by the need to keep the nanoparticle colloidally stable. Here reversible addition-fragmentation chain transfer (RAFT) synthesis has excelled due to a large variety of monomers and easy functionalization. The chain transfer agent (CTA) can be readily used as functional group itself, a functionalized CTA can be used or the polymer chains can be post-functionalized. A chemical reaction or physisorption is used to attach the polymers to the nanoparticle. One drawback of grafting-to is the usually lower grafting density, due to the steric repulsion of the coiled polymer chains during attachment to the reference particle surface. All grafting-to methods suffer from the drawback that a rigorous workup is necessary to remove the excess of free ligand from the functionalized nanocomposite reference particle. This is typically achieved by selective precipitation and centrifugation. In the grafting-from approach molecules, like initiators for atomic transfer radical polymerization (ATRP) or CTAs for (RAFT) polymerizations, are immobilized on the reference particle surface. The drawbacks of this method are the development of new initiator coupling reactions. Moreover, contrary to grafting-to, the reference particles have to be colloidally stable under the polymerization conditions.

Reference Particles Comprising Polymers with Metal-Chelating Groups

Another means for generating reference particles for use in the invention is to use a polymer particle wherein the polymer comprises metal-chelating ligands attached to at least one subunit of the polymer. The number of metal-chelating groups capable of binding at least one metal atom in the polymer can be between approximately 1 and 10,000, such as 5-100, 10-250, 250-5,000, 500-2,500, or 500-1,000. At least one metal atom can be bound to at least one of the metal-chelating groups. The polymer can have a degree of polymerization of between approximately 1 and 10,000, such as 5-100, 10-250, 250-5,000, 500-2,500, or 500-1,000.

The metal-chelating group that is capable of binding at least one metal atom can comprise at least four acetic acid groups. For instance, the metal-chelating group can be a diethylenetriaminepentaacetate (DTPA) group or a 1,4,7,10-tetraazacyclododecane-1,4,7,10-tetraacetic acid (DOTA) group. Alternative groups include Ethylenediaminetetraacetic acid (EDTA) and ethylene glycol-bis((3-aminoethyl ether)-N,N,N′,N′-tetraacetic acid (EGTA).

In certain embodiments, the polystyrene of the polystyrene beads for use in the invention may further comprises metal-chelating groups. For example, the polystyrene bead may comprise a polystyrene-polyacrylate copolymer, a polystyrene-polyacrylamide copolymer, a polystyrene-polymethacrylate copolymer, or a polystyrene-polymethacrylamide copolymer; wherein each metal chelating group is attached to a polymer subunit derived from the polyacrylamide, polymethacrylate, or polymethacrylamide.

In certain embodiments, the metal-chelating group can be attached to the polymer through an ester or through an amide. Examples of suitable metal-chelating polymers include the X8 and DM3 polymers available from Fluidigm Canada, Inc. A strategy of preparing polymers containing functional pendant groups in the repeat unit to which the ligated transition metal unit (for example a Ln unit) can be attached in a later step can be adopted. This embodiment has several advantages. It avoids complications that might arise from carrying out polymerizations of ligand containing monomers.

In certain embodiments, the metal-chelating polymers for use in the invention may comprise polycyclopropanes accessed through the ROP of cyclopropane. The cyclopropane monomer can comprise substituents capable of substitution with metal-chelating groups. For example, ROP of cyclopropane-1,1-dicarboxylates, reported by Illy, N. et al., Macromol. Rapid Comm., 2009, 30, 1731-1735, provides polycyclopropane bearing two carboxylate groups per repeat unit.

In certain embodiments, the metal-chelating polymers for use in the invention may comprise polynorbornene accessed via the ring-opening metathesis polymerisation (ROMP) of norbornene, for example using a Grubb's catalyst as known by those skilled in the art. The norbornene monomer may be substituted with substituents capable of substitution with metal-chelating groups. For example, 5-norbornene-2-carboxylate can undergo ROMP to provide polynorbornene bearing a carboxylate group on each repeat unit. In addition, other substituent cyclic ene and diene monomers can also be used to prepare metal-chelating polymers for use in the present invention. For example, 1,5-cyclooctadiene.

The metal-chelating groups can be attached to the polymer by methods known to those skilled in the art, for example, the pendant group may be attached through an ester or through an amide. For instance, to a methylacrylate based polymer, polycyclopropane, or polynorbornene bearing carboxylate groups, the metal-chelating group can be attached to the polymer backbone first by reaction of the polymer with ethylenediamine in methanol, followed by subsequent reaction of an excess of DTPA with a moderate excess of 4-(4,6-dimethoxy-1,3,5-triazin-2-yl)-4-methyl-morpholinium chloride (DMTMM). Such a methodology is discussed in Majonis, D., et al. Anal Chem., 2010, 82, 8961-9. Those skilled in the art will appreciate that any polymer bearing a reactive moiety which can be substituted with a diamine, for example ethylenediamine, can be functionalised with DOTA or DTPA using these methods. Such moieties include carboxylates, alkyl chlorides, acyl chlorides, or epoxides for example glycidyl groups.

In certain embodiments, the metal-chelating polymers for use in the invention may comprise polysiloxanes, obtained for example from the ring-opening polymerisation (ROP) of cyclic trisiloxanes, as known by those skilled in the art. ROP of cyclic trisiloxanes bearing vinyl substituents on silicon, for example the methylvinylsiloxane cyclic trimer, provides polysiloxanes comprising vinyl groups on every silicon atom in the backbone. Metal chelating groups can then be substituted onto the backbone of the polymer using click chemistry with reagents comprising thiol groups, as known by those skilled in the art.

In certain embodiments, the metal-chelating polymers for use in the invention may comprise polyphosphazenes, obtained, for example, from the ring-opening polymerisation (ROP) of cyclic triphosphazenes, as known by those skilled in the art. ROP of cyclic triphosphazenes bearing two chloro substituents on each phosphorus, for example the hexachlorophosphazene, provides polyphosphazenes comprising two chloro groups on every phosphorus atom in the backbone. Metal chelating groups can then be substituted onto the backbone of the polymer by substituting the chloro substituents with metal-chelating reagents comprising a nucleophilic atom capable of substituting the chloro substituent, e.g. an alcohol or amine. Alternatively, the chloro substituents could be substituted with a nucleophilic reagent comprising a vinyl group, for example an allyl alcohol or allyl amine, to provide polyphosphazenes bearing vinyl groups on each phosphorus atom in the backbone of the polymer. Metal chelating groups can then be substituted onto the backbone of the polymer using click chemistry with reagents comprising thiol groups, as known by those skilled in the art. In some embodiments, polypeptides are functionalised with metal chelating groups such as DOTA, DPTA, or EDTA using the methods discussed herein, or are polypeptides or proteins which are otherwise capable of binding to metals (e.g. naturally occurring or engineered metal-binding peptides, polypeptides and proteins). For example, the terminal alkyl amine group present on each repeat unit of a polylysine can be functionalised with one of the metal-chelating groups, to provide a polypeptide capable of binding a lanthanide. Examples of such functionalisation maybe found in Haung, Z., et al., RSC Adv. 2018, 8, 5005-5012. In addition, functionalisation of polyglutamide with DTPA has been reported in Lu, Y., et al., Biomacromolecules, 2014, 15, 2027-2037.

The degree of substitution of the polymer backbone with the metal-chelating groups can be carefully controlled through the selection of appropriate reagent stoichiometry, e.g. when using click chemistry to attach the metal chelating groups to vinyl groups on the backbone of a polymer. Thus, in some embodiments at least 20% of the repeat units of the metal-chelating polymers for use in the invention have metal-chelating groups bound thereto, for example at least 30, at least 50, at least 70, at least 80, at least 90, at least 99%, or substantially all of the repeat units have metal-chelating groups bound thereto.

The metal-chelating polymers may be incorporated into reference particles for use in the invention. In some embodiments, the metal-chelating polymers are incorporated onto the surface of particles, in other words the surface of a particle is functionalised with a metal-chelating polymer to create a 3D polymer brush. The polymers can either by grown directly from the surface of the particles (grafting from), alternatively pre-formed metal-chelating polymers may be attached to the surface of the particles (grafting to).

The particles will be of a size such that surface functionalisation with a metal-chelating polymer provides a sufficient number of reference atoms for accurate detection, and subsequent normalisation and calibration of the detected signal intensity. Accordingly, in some embodiments, the particles from which an SAM comprising metal-chelating or metal-containing polymers will have a longest diameter between 0.2 μm to 20 μm, including 0.5 μm to 10 μm, 1 μm to 5 μm, 1 μm to 3 μm, or 1 μm to 2 μm, or about 1 μm.

### Reference Particles Comprising Metal-Containing Polymers

A yet further means of making reference particles for use in the invention is to generate polymers that include the reference atom in the backbone of the polymer rather than as a co-ordinated metal ligand. For instance, Carerra and Seferos (Macromolecules 2015, 48, 297-308) disclose the inclusion of tellurium into the backbone of a polymer. Other polymers incorporating atoms capable as functioning as reference atoms tin-, antimony- and bismuth-incorporating polymers. Such molecules are discussed inter alia in Priegert et al., 2016 (Chem. Soc. Rev., 45, 922-953).

To conclude, reference particles for use in the invention can comprise at least two components: the reference atoms, and a polymer, which either chelates, contains or is doped with at least one reference atom.

Polymers for use in the invention may be amenable to synthesis by a route that leads to a relatively narrow polymer dispersity. For example, the polymer can be synthesised from the group consisting of reversible addition fragmentation polymerization (RAFT), atom transfer radical polymerization (ATRP), nitroxide-mediated polymerisation (NMP), and photoiniferter-mediated polymerisation (PIMP), which should lead to values of Mw (weight average molecular weight)/Mn (number average molecular weight) in the range of 1.1 to 1.2. In addition single electron living radical polymerisation where polymers with Mw/Mn of approximately 1.02 to 1.05 are obtainable. These methods permit control over end groups, through a choice of initiating or terminating agents. This allows synthesizing polymers to which linkers can be attached. Furthermore, these methods permit the synthesis of block copolymers through sequential monomer addition.

Polymers that be used to make the reference particles for use in the invention also include:
random copolymer poly(DMA-co-NAS): The synthesis of a 75/25 mole ratio random copolymer of N-acryloxysuccinimide (NAS) with N,N-dimethyl acrylamide (DMA) by RAFT with high conversion, excellent molar mass control in the range of 5000 to 130,000, and with Mw/Mn=1.1 is reported in Relógio et al. (2004) (Polymer, 45, 8639-49). The active NHS ester is reacted with a metal-chelating group bearing a reactive amino group to yield the metal-chelating copolymer synthesised by RAFT polymerization.
poly(NMAS): NMAS can be polymerised by ATRP, obtaining polymers with a mean molar mass ranging from 12 to 40 KDa with Mw/Mn of approximately 1.1 (see e.g. Godwin et al., 2001; Angew. Chem. Int. Ed, 40: 594-97).
poly(MAA): polymethacrylic acid (PMAA) can be prepared by anionic polymerization of its t-butyl or trimethylsilyl (TMS) ester.
poly(DMAEMA): poly(dimethylaminoethyl methacrylate) (PDMAEMA) can be prepared by ATRP (see Wang et al, 2004, J. Am. Chem. Soc, 126, 7784-85). This is a well-known polymer that is conveniently prepared with mean Mn values ranging from 2 to 35 KDa with Mw/Mn of approximately 1.2 This polymer can also be synthesized by anionic polymerization with a narrower size distribution.
polyacrylamide, or polymethacrylamide.

The metal-chelating groups can be attached to the polymer by methods known to those skilled in the art, for example, the pendant group may be attached through an ester or through an amide. For instance, to a methylacrylate based polymer, the metal-chelating group can be attached to the polymer backbone first by reaction of the polymer with ethylenediamine in methanol, followed by subsequent reaction of an excess of DTPA with a moderate excess of 4-(4,6-dimethoxy-1,3,5-triazin-2-yl)-4-methyl-morpholinium chloride (DMTMM). Such a methodology is discussed in Majonis, D., et al. Anal Chem., 2010, 82, 8961-9. Alternatively, the polymer functionalised with ethylenediamine can be subsequent reacted with DTPA anhydride under alkaline conditions in a carbonate buffer.

### Sample Carrier Substrates

When reference particles are to be fused to a sample carrier by heating, the sample carrier substrate can be any solid phase that retains its structural integrity at the temperatures required to fuse the reference particles to it. In other words, whilst different combinations of sample carrier and reference particle can be used in the present invention, it will be understood by one of skill in the art that the sample carrier substrate cannot be made of any material with a glass transition or melt temperature less than the maximum temperature employed to fuse the particulates to the surface of the sample carrier. Provided this criterion is met, sample carriers used in the present invention can be modified as discussed herein.

When reference particles are to be fused to a sample carrier by the use of a solvent to partially solvate or swell the reference particles, the sample carrier substrate can be any solid phase that retains its structural integrity in the solvents used to fuse the reference particles to it. In other words, whilst different combinations of sample carrier and reference particle can be used in the present invention, it will be understood by one of skill in the art that the sample carrier substrate cannot be made of any material with a Hildebrand solubility parameter closer to the solvent being used for fusion than the reference particles. Provided this criterion is met, sample carriers used in the present invention can be modified as discussed herein.

Examples of materials for sample carriers that may be used in the present invention include glass, silica, aluminium, cellulose, chitosan, Indium Tin Oxide (ITO), Aluminium oxide (Al2O3), Magnetite (Fe₃O₄), CuOx, Hematite (c-Fe₂O₃), Manganese spiral Ferrite (MnFe₂O₄), Magnesium hydroxide (Mg(OH)₂), Zinc oxide (ZnO), zirconium phosphonate, halloysite, montmorillonite, steel, sapphire, Cadmium selenide (CdSe), Cadmium sulphide (CdS), Gallium Arsenide (GaAs), mica, carbon black, diamond, single walled carbon nanotubes, multiwalled carbon nanotubes, graphene, and encompass planar surfaces in the form, e.g., of microscope slides.

Where the sample carrier is planar, then it may be optically transparent, for example made of glass. Where the sample carrier is optically transparent, it enables ablation of the sample material through the support. For example, the solid support may include a tissue slide. Through-carrier ablation is discussed for example in WO2014169394. Planar sample carriers may also contain elemental coding in the substrate.

### Methods of Making an Imaging Mass Calibrator

The imaging mass calibrator is generated by fusion of particles to a sample carrier. First the particles are dispersed onto the sample carrier and then they are fused to the carrier.

Contacting the Sample Carrier with a Suspension of Reference Particles

The first step in the method of the present invention comprises contacting the sample carrier with a suspension of at least one reference particle wherein the at least one reference particle comprises at least one reference atom.

Any solvent can be used that suspends the reference particles can be used in the methods of the present invention, i.e. not solvents that dissolve the reference particles. Those skilled in the art will appreciate that the nature of the reference particles will dictate which solvents can be employed to form the suspension used in the method of the present invention. For example, wherein the reference particles are metal-doped polystyrene beads, the reference particles may be suspended in water, ethanol, other alcohols and solvents, for example, methanol, propanol, butanol, acetone, acetic acid, mixtures thereof and the like.

Normalisation of the signal intensity and/or calibration of an imaging mass cytometer may be best performed when an entire reference particle of known elemental composition and amount can be ablated from the sample carrier and the integral signal intensity for each reference atom associated with the reference particle determined and compared to the expected integral signal intensity for the reference particle based on the amount of the reference atoms known to be present (normalisation and calibration of the detector using the imaging sample carrier of the invention is discussed further on the section starting of page 44). Thus, it is desirable that individual reference particles be fused onto the sample carrier discretely, i.e. they do not aggregate on the surface of the sample carrier, therefore allowing accurate determination of an average integral signal intensity per reference particle. Reference particles must therefore be contacted with the sample carrier in a way that prevents significant agglomeration of the reference particles and thus allows discrete reference particles to be fused onto the sample carrier. For example, contacting the sample carrier with a suspension of reference particles could allow up to 2%, 5%, 8%, 10%, 15%, or 20% of the reference particles to be agglomerated on the sample carrier of the imaging mass calibrator of the invention.

Accordingly, in some embodiments the method of the present invention further comprises contacting the sample carrier with a suspension of at least one reference particle, wherein the reference particles are present in a concentration that ensures both adequate coverage on the sample carrier, such that the user may easily locate reference particles for ablation during normalisation and calibration, and that individual reference particles can be fused to the sample carrier discreetly. Furthermore, that the particles are present in sufficient number to allow for the sampling of the number of particles necessary for the calibration and normalisation of the detected signal intensity during the imaging of the sample. Adequate coverage of the sample carrier is that required to ensure that the above conditions are met.

In some embodiments, after contacting the sample carrier with a suspension comprising the reference particles, the method of the invention comprises spreading the suspension across an areas of the sample carrier, for example a 1 mm×1 mm, 2 mm×2 mm, 4 mm×4 mm, 6 mm×6 mm, 8 mm×8 mm, a 10 mm×10 mm or at 10 mm×20 mm area of the sample carrier. Spreading the suspension of reference particles over an area of the sample carrier will increase the rate of evaporation of the solvent, in addition may assist in providing a sufficient number of the reference particles isolated discretely on the sample carrier, i.e. reduce the amount of agglomeration of the reference particles.

Those skilled in the art will appreciate that the concentration of reference particles in the suspension that is required to ensure both adequate coverage of the at least one reference particle on the sample carrier and fusing of individual reference particles in isolation will be dependent on the nature of both the reference particles and the solvent employed, and can be easily determined through routine experimentation. For example, when the particles are metal-doped polystyrene beads of 3 μm diameter in a suspension of water or EtOH, covering an area of the sample carrier, for example 1 mm×1 mm, 2 mm×2 mm, 4 mm×4 mm, 6 mm×6 mm, 8 mm×8 mm, a 10 mm×10 mm or at 10 mm×20 mm area of the sample carrier with a suspension of particles at a concentration of between 1×10³ to 1×10¹⁵ particles per ml, for example from 1×10⁶ to 1×10⁸ particles per ml, 1×10⁵ to 1×10⁹ particles per ml, 1×10⁶ to 1×10⁸ particles per ml, 1×10⁶ to 1×10⁸ particles per ml, or about 9×10⁷ particles per ml provides a concentration of at least 1, at least 2, at least 3, at least 5, at least 8, at least 10, at least 15, at least 20 particles per 100 μm×100 μm area of sample carrier. For example, wherein the reference particles are metal-doped polystyrene EQ4 beads, pipetting 2 μl of a suspension of the beads at a concentration of 1×10⁶ to 1×10⁸ particles per ml in both EtOH and water was found to provide an adequate coverage of the sample carrier with individually isolated beads, e.g. about 12 particles per 100 μm×100 μm area of the sample carrier, and about 1.7×10⁵ beads in a 1×1 cm area of the sample carrier.

The suspension of the particles may be pipetted onto the sample carrier. In some embodiments at least 1 μl, at least 2 μl, at least 3 μl, at least 4 μl, at least 5 μl, at least 8 μl, or at least 10 μl of the suspension of the particles is pipetted onto the slides. In some embodiments, the suspension of the reference particles is pipetted substantially away from the edge of the sample carrier. For example, the suspension of the reference particles is pipetted at least 1 mm, at least 2 mm, at least 3 mm, at least 4 mm, at least 5 mm, from the edge of the slide. Those skilled in the art will appreciate that positioning the particles away from the edge of the slide will reduce variation in the integral signal intensity detected that may result from the particles being located near the edge of the slide (i.e. “edge effects”).

In some embodiments, the method comprises contacting the sample carrier with a suspension comprising at least one reference particle, wherein the at least one reference particle comprises more than one different reference atom, for example at least two, three, four, five, six, seven, eight, or at ten different labelling atoms. For example, each at least one reference particle comprises a mixture of different reference atoms. Thus, the method may provide an imaging mass calibrator that can be used to calibrate and normalise the signal intensity for multiple mass channels of the detector of an imaging mass cytometer.

In some embodiments, the method comprises contacting the sample carrier with a suspension comprising more than one reference particle, for example at least two, three, four, five, six, seven, eight, or at least ten sets of at least one reference particle, wherein each set of at least one reference particle comprises a different reference atom. Thus, the method may provide an imaging mass calibrator comprising different sets of at least one particle located in discrete regions of the sample carrier, which can be used to easily identify the different mass channels of the detector of an imaging mass cytometer for calibration and normalisation of the signal intensity. Accordingly, in some embodiments, the method comprises contacting the sample carrier with a suspension comprising more than one reference particle, for example at least two, three, four, five, six, seven, eight, or at least ten sets of at least one reference particle, wherein each set of at least one reference particle comprises a different reference atom and wherein each set of reference particles is contacted with a different discrete region of the sample carrier.

In some embodiments, the method comprises contacting the sample carrier with a suspension comprising more than one reference particle, for example at least two, three, four, five, six, seven, eight, or at least ten sets of at least one reference particle, wherein each set of at least one reference particle comprises a different amount of the same reference atom. Thus, the method may provide an imaging mass calibrator that can be used to plot a calibration curve fora mass channel of the detector of an imaging mass cytometry (see page 48 for discussion of calibration curves). Accordingly, in some embodiments, the method comprises contacting the sample carrier with a suspension comprising more than one reference particle, for example at least two, three, four, five, six, seven, eight, or at least ten sets of at least one reference particle, wherein each set of at least one reference particle comprises a different amount of the same reference atom, and wherein each set of reference particles is contacted with a different discrete region of the sample carrier.

In some embodiments, the method comprises contacting the sample carrier with a suspension comprising more than one reference particle, for example at least two, three, four, five, six, seven, eight, or at least ten sets of at least one reference particle, wherein each set of reference particles comprises multiple different reference atoms, wherein each set of at least one fused reference particle comprises a different amount of the multiple different reference atoms. For example, each set of reference particles may comprise the same mixture of reference atoms, but at different amounts. Thus, the method may provide an imaging mass calibrator that can be used to plot calibration curves for multiple different mass channels of the detector of an imaging mass cytometry (see page 48 for discussion of calibration curves). Again, the different sets of reference particles may be located in discrete regions. Accordingly, in some embodiments, the method comprises contacting the sample carrier with a suspension comprising more than one reference particle, for example at least two, three, four, five, six, seven, eight, or at least ten sets of at least one reference particle, wherein each set of reference particles comprises multiple different reference atoms, wherein each set of at least one fused reference particle comprises a different amount of the multiple different reference atoms, and wherein each set of reference particles is contacted with a different discrete region of the sample carrier.

As discussed, isolating different sets of reference particles to discrete regions of the sample carrier allows for ease of identification of the respective set of reference particles based on their location on the sample carrier. Thus, different sets of particles comprising different specific reference atoms, combination of reference atoms, and/or amount of reference atoms can easily be identified, facilitating plotting of calibration curves for multiple different mass channels of the detector of an imaging mass cytometry. Methods for plotting calibration curves are discussed herein (see page 48)

In some embodiments, the method of the invention further comprises the step of, after contacting the sample carrier with a suspension comprising at least one reference particle, drying the sample carrier. The sample carrier can be left to dry in air at room temperature. Alternatively, the sample carrier can be dried by heating the sample carrier at a temperature below the boiling point of the solvent. For example the sample carrier may be heated to 10° C., 20° C., 30° C., 40° C., 50° C. below the boiling point of the solvent. Heating the sample carrier to evaporate the solvent should be conducted at a temperature which does not result in bubbling or boiling of the solvent which may result in the agglomeration of the particles on the slide. The sample carrier may be inspected by an optical microscope to determine that the solvent has evaporated.

### Fusing Reference Particles to a Sample Carrier

The method of making the imaging mass calibrator of the invention further comprises the step of, once the sample carrier has been contacted with a suspension comprising at least one reference particle, fusing the at least one reference particle with the sample carrier. The reference particles can be fused to the sample carriers by a variety of methods.

As noted above, in some instances, multiple sets of particles can be contacted with different discrete areas of the sample carrier. In some embodiments of the invention, all of the different sets of reference particles are contacted with the different discrete areas before all reference particles are fused to the sample carrier in a single fusion step.

### Fusion by Heating

The step of fusing the at least one reference particle to sample carrier may comprise heating the sample carrier. The method of the invention may further comprise the additional step of, before fusing the at least one particle with the sample carrier, drying the sample carrier. In some embodiments, the step of fusing the at least one reference particle with the sample carrier comprises heating the sample carrier at a temperature above the glass transition temperature of the reference particle and subsequently cooling the sample carrier below the glass transition temperature of the reference particle. In other words the fusing of the at least one reference particle to the sample carrier can occur by vitrification. In some embodiments of the invention, the at least one reference particle may be crystalline such that the at least one reference particle is fused to the sample carrier by heating the sample carrier above the melt temperature of the at least one reference particle. In other words, the at least one reference particle is melted onto the sample carrier.

In some embodiments of the invention, the at least one reference particle has a glass transition temperature of at least 80° C., such as at least 100° C., at least 120° C., at least 140° C., at least 160° C., at least 180° C., or at least 200° C., such that heating of the sample carrier to fuse the at least one reference particle thereto is performed above said temperatures. In some embodiments, the sample carrier is heated to a maximum of up to 300° C., for example up to 275° C., up to 250° C., up to 225° C. or up to 200° C. Heating the at least one reference particle above its glass transition temperature changes the at least one reference particle from its glass state to a viscous, rubbery state. Once the at least one reference particle is in the viscous, rubbery state it can fuse to the sample carrier. For example, wherein the at least one reference particle is a polymer bead, heating the at least one reference particle above its glass transition temperature provides sufficient energy to the polymer chains to overcome the energy barrier to conformational rotation such that the chains can slide past each other and adopt new conformations, thus adhering to the sample carrier.

The extent of the fusing of the particles to the sample carrier can be assessed by optical microscopy. Thus, following heating of the sample carrier, the sample carrier can be inspected under an optical microscope and the diameter of the fused particles compared to the diameter of the particles before heating. Without being bound by theory, when the particles are heated above their T_{g} and become viscous and rubbery, the particles will lose their spherical shape and become fused to the sample carrier. When in the viscous, rubbery state, the spherical reference particles will become flatter (e.g. a domed shape). The particles are considered to be sufficiently fused to the sample carrier when they are observed to increase in size from the size of the unfused particle. Inspection of the fused particles can be performed by optical microscopy. In some embodiments, the sample carrier is heated until the diameter of the particle is at least 5%, such at least 10%, at least 15%, at least 20%, at least 30%, at least 50%, at least 75%, at least 100%, or at least 150% greater in size than unfused reference particles.

It will be apparent to those skilled in the art that whilst heating the at least one reference particle slightly above its T_{g} (for example 5° C.) will facilitate the conformational changes in the reference particle required to fuse the at least one reference particle to the sample carrier, should the at least one reference particle be heated at temperatures in excess of the T_{g}, of the reference particle, more energy will be provided (e.g. to the polymer chains), facilitating faster conformational rearrangement (e.g. of the chains) and thus faster fusing of the at least one reference particle to the slide. Thus, in some embodiments the step of fusing the at least one reference particle to the sample carrier in the method of the present invention comprises heating the sample carrier and reference particle at temperatures in excess of the T_{g} of the reference particle, for example at least 10° C., at least 20° C., at least 30° C., at least 40° C., at least 50° C., at least 60° C., at least 80° C., or at least 100° C. in excess of the T_{g} of the reference particle. Furthermore, it will also be apparent to those skilled in the art that the time required for fusing the reference particles can be reduced when the temperature at which the reference particle is heated is increased in excess of the T_{g} of the reference particle. For example, those skilled in the art will appreciate that the higher the temperature at which the reference particle is heated above its T_{g}, the shorter the duration of time that will required to fuse the reference particle to the slide.

For example, in some embodiments, the method of the invention comprises heating the sample carrier and reference particle at least 20° C. above the T_{g} for 60 minutes, at least 50° C. above the T_{g} for 30 minutes, at least 60° C. above the T_{g} for 10 minutes, at least 70° C. above the T_{g} for 30 minutes, at least 70° C. above the T_{g} for 20 minutes, at least 100° C. above the T_{g} for 10 minutes, at least 100° C. above the T_{g} for 5 minutes. It will be appreciated by those skilled in the art that the selection of the temperature of heating in excess of the T_{g} of the reference particle, in addition to the duration of heating, will be made to both ensure that thermally-induced damage to the sample does not occur and that the fusion of the reference particles to the sample carrier occurs within a time which is not impractical to the user.

In some embodiments, the method of the present invention comprises fusing a set of at least one reference particle to the sample carrier to a discrete area of the sample carrier, before contacting the sample carrier with an additional suspension comprising a further set of at least one reference particle, wherein each set of at least one reference particle can comprise a different reference atom, a different amount of the same reference atom, or the same mixture of reference atoms but at different amounts. Said method of contacting the sample carrier with a set of at least one reference particle and fusing said at least one reference particle to the sample carrier could be repeated until two, three, four, five, six, seven, eight, or at least ten distinct areas of sample carrier have been formed, wherein each area may comprise a different reference atom or a different amount of the same reference atom.

In some embodiments, the method of the invention comprises fusing more than one set of reference particles to the sample carrier, wherein each set of reference particle has a different T_{g}. Thus, in some embodiments the method comprises contacting the sample carrier with a suspension comprising a first set of at least one reference particle, heating the first set of at least one reference particle above its T_{g}, contacting the sample carrier with at least one additional set of at least one particle, wherein the additional set of at least one particle has a different T_{g} to the first set of at least one particle, and heating the at least one additional set of at least one particle above its T_{g}. As will be appreciated by one of skill in the art, the steps of serial additions should start with the reference particles of the highest T_{g}, followed by the next highest and so on, so that progressively lower temperatures can be used to fuse the second and subsequent particles to the sample carrier. The method may further comprise washing the sample between each heating step.

It will also be apparent to those skilled in the art that adaptation of the method of fusing the at least one reference particle to the slide may be required depending on the nature of the reference particle and the sample being imaged. For example, those skilled in the art will appreciate that the reference particle cannot be heated at a temperature at which the sample carrier itself will lose structural integrity, for example above the melt temperature of material of the sample carrier. Accordingly, the fusing step in the method of the invention is performed at a temperature below the T_{g} of the sample carrier.

### Solvent-Based Fusion

The inventors have discovered an alternative method for fusing the reference particles to the imaging mass calibrator of the invention that can be performed be employing a solvent to partially solvate or swell the reference particles. Such a technique will find particular utility when a user requires the fusing of reference particles to sample carriers that already comprise thermally-sensitive samples, that would be adversely affected should the reference particles be fused to the sample carrier using heat.

The inventors have thus discovered that partial solvation of the polymer reference particles facilitates fusion of the particles to the sample carrier. Without wishing to be bound by theory, it is believed that solvents or mixtures of solvents capable of at least partially solvating the polymer particles (i.e. “fusion” solvents) permeate into the matrix of polymer chains and plasticize the polymer chains to reduce the energy barrier for conformational change within the polymer matrix. Thus, the solvent effectively lowers the T_{g} of the reference particles to sub-ambient temperatures, for example below 25° C., such that the polymer chains can slide past each other and fuse to the surface of the slide. Once the solvent has evaporated, the reference particle is therefore fused to the sample carrier.

The amount and choice of “fusion” solvent or mixture of solvents comprising a “fusion” solvent for use in the method of the invention may be selected to ensure permeation of the solvent into the reference particles, in order to plasticize the polymer chains and induce fusion to the sample carrier, but not to solvate the reference particles to an extent that leaching of the reference atoms from the reference particles occurs. Absolute calibration and normalisation of the signal intensity detected in a mass cytometry/spectroscopy necessitates that each reference particle fused to the sample carrier has an identical and known elemental composition and amount. Leaching of the reference atoms from the reference particles obviously prohibits such absolute methods.

Solvents or mixtures of solvents may be selected based on their capacity to at least partially solvate the particular reference particle being used. One method of determining solvents suitable for use in the method of the invention is through a comparison of the Hildebrand solubility parameters for the polymer comprising the reference particles and the solvent. Should the Hildebrand solubility parameter for the solvent be similar to the reference particle, i.e. be a “fusion” solvent for the reference particle, this particular solvent will readily solvate the reference particle. Alternatively, should the Hildebrand solubility parameter of the solvent be dissimilar to the reference particle, i.e. be a “suspension” solvent for the reference particle, this particular solvent will not solvate or significantly permeate the reference particle.

Thus, in some embodiments, a solvent with a similar Hildebrand solubility parameter to the reference particle has a value of the parameter within at least 2 J^{1/2} m^{-3/2}, at least 1 J^{1/2} m^{-3/2}, at least 0.6 J^{1/2} m^{-3/2}, at least 0.4 J^{1/2} m^{-3/2}, at least 0.2 J^{1/2} m^{-3/2}, at least 0.1 J^{1/2} m^{-3/2}, or substantially the same Hildebrand solubility parameter. In some embodiments, a solvent with a dissimilar Hildebrand solubility parameter to the reference particle has a value of the parameter at least 2 J^{1/2} m^{-3/2}, at least 2.4 J^{1/2} m^{-3/2}, at least 3 J^{1/2} m^{-3/2}, at least 4 J^{1/2} m^{-3/2}, at least 6 J^{1/2} m^{-3/2}, or at least 10 J^{1/2} m^{-3/2} different to the value of reference particle.

In some embodiments of the invention, the “fusion” solvent or mixtures of solvent comprising a “fusion” solvent will have a Hildebrand solubility parameter between 0.02 and 10 J^{1/2} m^{-3/2} away from the Hildebrand solubility parameter of the reference particle, for example between 0.1 and 8 J^{1/2} m^{-3/2}, 0.2 and 6 J^{1/2} m^{-3/2}, 0.4 and 4 J^{1/2} m^{-3/2}, 0.2 and 2 J^{1/2} m^{-3/2}, 0.6 and 2 J^{1/2} m^{-3/2}, 0.8 and 1.6 J^{1/2} m^{-3/2} away from the Hildebrand solubility parameter of the reference particle,

For example, wherein the reference particles are metal-doped polystyrene beads (Hildebrand solubility parameter=18.68 J^{1/2} m^{-3/2}) a suitable solvent for partially solvating the reference particles is acetone (Hildebrand solubility parameter=19.9 J^{1/2} m^{-3/2}), whereas ethanol (Hildebrand solubility parameter=26.5 J^{1/2} m^{-3/2}) does not partially solvate the reference particles and ethyl acetate (Hildebrand solubility parameter=18.2 J^{1/2} m^{-3/2}) is a good solvent for the polystyrene reference particle and therefore its use may result in leaching of the reference atoms from the reference particles.

Solvents for use in the method of the invention include pentane, hexane, cyclohexane, heptane, octane, diethyl ether, ethyl acetate, chloroform, dichloromethane, acetone, toluene, methanol, ethanol, propanol, butanol, acetonitrile, tetrahydrofuran, xylene, dimethyl sulfoxide, acetone, acetic acid, water, mixtures thereof and the like. Volatile solvents for us in the method of the invention include pentane, hexane, cyclohexane, diethyl ether, chloroform, dichloromethane, mixtures thereof and the like.

In addition, mixtures of solvents may also be used to partially solvate the reference particles. For example, a mixture of at least two solvents may be used, wherein one solvent has a Hildebrand solubility parameter similar to the reference particle (i.e. a “fusion” solvent) and the other has a Hildebrand solubility parameter dissimilar to the reference particle (i.e. a “suspension” solvent). For example, wherein the reference particles are polystyrene reference particles, a mixture of ethyl acetate and ethanol may be used. The resulting mixture may partially solvate the reference particle, without significant leaching of the reference atoms. Those skilled in the art will appreciate that the specific ratio of the two solvents that can be used in the method of the invention will depend on the polymers comprising the reference particles and the solvents that form the mixture. Thus, in some embodiments, the mixture comprises 99 to 1% by weight of a “suspension” solvent to a “fusion” solvent, for example 98 to 2, 95 to 5, 90 to 10, 80 to 20, 70, to 30, 60 to 40, 60 to 50, 40 to 60, 30 to 70, 20 to 80, or 10 to 90% by weight of a “suspension” solvent to a “fusion” solvent.

Limiting the amount of time the particles spend in the presence of a “fusion” solvent reduces the likelihood of leaching of the reference atoms from the reference particles. In addition, methods that requires that the reference particles be suspended in a solution comprising a good solvent for a long period of time are not desirable as complete solvation of the reference particles may occur, such that the user obtains a solution comprising solvated polymer chains and solvated reference atoms, which cannot be used in the invention. It is therefore desirable to limit the amount of time the particles spend in a “fusion” solvent or mixtures of solvent comprising a “fusion” solvent, such that reference atoms will not leach from the particles to an extent that accurate absolute calibration and normalisation is no longer possible. Accordingly, the methods of the invention all limit the amount of time the reference particles spend in the presence of a “fusion” solvent.

A first method of solvent-fusing the reference particles to the imaging mass calibrator of the invention involves dispersing the reference particles onto the sample carrier by contacting the sample carrier with a suspension of the reference particles in a solvent that does not solvate the particles, i.e. a poor solvent, in an identical method to the first method described above. Once the reference particles have been dispersed onto the sample carrier and dried, the reference particles are then exposed to a solvent vapour. The solvent or mixtures of solvents that have been vaporised and introduced to the reference particles comprise a solvent capable of at least partially solvating the reference particles, i.e. a “fusion” solvent. In the method of the invention, the solvent vapour permeates the polymer matrix of the reference particles, plasticizes the polymer chains such that polymer chains can fuse to the sample carrier. The solvent vapour that has permeated the reference particle then evaporates. In other words, solvent annealing may be used to fuse the reference particles onto the sample carrier.

It will be appreciated by those skilled in the art that solvents will be selected according to both their relative Hildebrand solvent parameter (i.e. relative to the reference particle in question) and their volatility. Highly volatile solvents will vaporize at ambient temperatures to provide a solvent vapour without heating of the sample. For example, wherein the reference particles comprise polystyrene, polymethylmethacrylate, or copolymers thereof, dichloromethane (DCM) (vapour pressure at 25° C.=58 kPa) or chloroform (vapour pressure at 25° C.=30 kPa) may be used to form a solvent vapour which permeates the particles and fuses them to the sample carrier. In some embodiments the solvent used to solvent anneal the reference particles has a vapour pressure at 25° C. of at least 10 kPa, for example at least 20, at least 30, at least 40, at least 50 kPa at 25° C.

This method reduces the possibility of any leaching of the reference atoms from the reference particles, as the solvent vapour can permeate the particles but does not provide a medium for the leaching of the reference atoms out of the reference particles onto other areas of the sample carrier.

A second method of solvent-fusing the reference particles to the imaging mass calibrator of the invention involves dispersing the reference particles onto the sample carrier by contacting the sample carrier with a suspension of the reference particles in a solvent that does not solvate the particles, i.e. a “suspension” solvent. For example, if the reference particles are metal-doped polystyrene beads, any of the solvents used to disperse the reference particles on the slide described in the section being on page 23 can be employed, e.g. water or ethanol, or mixtures thereof. Once the particles have been dispersed onto the slide and the sample carrier dried, an amount of “fusion” solvent or mixture of solvents comprising a “fusion” solvent is added to the dispersed reference particles on the sample carrier. The reference particles are then plasticised and fuse to the sample carrier as the solvent or mixture of solvent first permeates the reference particles then evaporates. Although the sample carrier may be heated to aid evaporation of the solvent, it will be appreciated that this may not be desirable should the sample carrier comprise a thermally-sensitive sample.

The solvent can be selected according to its volatility. Solvents of higher volatility will evaporate faster from the sample carrier, thus limiting the time the reference particle is in contact with said solvent. Thus, solvents or mixtures of solvents comprising a solvent with a Hildebrand solubility parameter more similar to the reference particles may be used if the solvents or mixtures of solvents are highly volatile. Solvents or mixtures of solvents of higher volatility can also be used in greater quantities.

Thus, in some embodiments of the method of the invention, at least 2 μl, at least 3 μl, at least 4 μl, at least 5 μl, at least 10 μl, at least 15 μl, at least 20 μl, at least 50 μl, at least 100 μl of a “fusion” solvent or mixture of solvents comprising a “fusion” solvent is added to the reference particles dispersed on the sample carrier.

A third method of solvent-fusing the reference particles to the imaging mass calibrator of the invention involves using a solvent comprises the step of adding an amount of a “fusion” solvent, i.e. a solvent with a similar Hildebrand solubility parameter to the reference particles, to a suspension of the particles in a “suspension” solvent, i.e. a solvent with a dissimilar Hildebrand solubility parameter to the reference particles, before contacting the sample carrier with the suspension. The selection of the identity and amount of the “fusion” solvent to be added to the “suspension” solvent is made depending on the respective identities of the reference particle and the solvents. Thus, in some embodiments the resulting mixture of solvents comprise 99 to 1% by weight of a “suspension” solvent (dissimilar Hildebrand solubility parameter) to a “fusion” solvent (similar Hildebrand solubility parameter), for example 98 to 2, 95 to 5, 90 to 10, 80 to 20, 70, to 30, 60 to 40, 60 to 50, 40 to 60, 30 to 70, 20 to 80, or 10 to 90% by weight of a “suspension” solvent to a “fusion” solvent. The method of contacting the sample carrier with the suspension comprising the reference particles can then be performed as detailed in the section beginning on page 23.

As stated, more volatile solvents evaporate more quickly from the sample carrier and thus are in contact with the reference particles for a shorter period of time, thus a more volatile mixture of the solvents may comprise more of the “fusion” solvent.

Any of the methods described above can be used to solvent-fuse reference particles such as those discussed in the section beginning on page 7. An additional type of reference particle particularly amenable to solvent-fusion is crosslinked polymer particles. Such particles comprise a crosslinked matrix of polymer chains. Thus, the addition of a solvent, mixtures of solvent, or solvent vapour, may not solvate the polymer chains, no matter how much of a “fusion” solvent is added and for what period. Instead, the addition of “fusion” solvent or mixture of solvents comprising a “fusion” solvent swells the cross-linked reference particles, allowing the particles to fuse to the sample carrier as the solvent evaporates and the particles contract.

Thus, cross-linked polymer particles will not be fully solvated either when in a suspension, e.g. if the reference particles are introduced into a “fusion” solvent or mixture of solvents comprising a “fusion” solvent prior to contacting the sample carrier (i.e. the second method described above)), or on the sample carrier itself, e.g. should a “fusion” solvent or mixture of solvents comprising a “fusion” solvent contact the reference particles once dispersed on the slide (i.e. the first and third methods described above). Use of crosslinked particles may therefore reduce leaching of the reference atoms from the reference particles.

Whilst the method of the invention reduces leaching of reference atoms, it will be appreciated that contacting cross-linked polymer reference particles with a “fusion” solvent or mixture of solvents comprising a “fusion” solvent for long periods of time may still result in leaching of the reference atoms. Thus, the methods of solvent-fusing the cross-linked polymer reference particles to the sample carrier preferably still limit the time the reference particle is in the presence of the “fusion” solvent or mixture of solvents comprising the “fusion” solvent.

### Sample Preparation for Imaging Mass Calibrators

Should the sample carrier used in the method of making an imaging mass calibrator of the invention already comprise a sample, for example a biological sample, it will be apparent to those skilled in the art that optimisation of the conditions for fusing the reference particle may be necessary to minimise thermal damage to the sample that could occur. Those skilled in the art will appreciated that said optimisation will involve finding a compromise between the temperature at which the at least one reference particle is heated and the duration of time for which the at least one reference particle is heated. In other words, those skilled in the art will optimise the method of the invention, employing a heating temperature that ensures rapid fusing of the reference particles to the sample carrier, such that time the sample is heated can be reduced, but that does not thermally damage the sample or affect the signal intensity associated with the sample that is detected during imaging, or any damage is limited to an acceptable level.

In some embodiments, the method of the invention further comprises, before contacting the sample carrier with a suspension comprising at least one reference particle, the additional steps of loading a sample on the sample carrier, and labelling the sample with a labelling solution comprising at least one mass-tagged SBP. In some embodiments, the method further comprises washing the sample, and drying the sample. The sample may be a biological sample that may include the other half (or halves) of the at least one specific binding pairs, wherein said half of a specific binding pair is complementary to a half of a specific binding pair in the staining solution.

Biological samples used in imaging mass cytometry and imaging mass spectrometry may comprise materials from prior stages in sample processing. For example, to generate tissue sections, often the tissue is fixed in paraffin wax. Said materials require removal before the sample can be labelled with mass-tagged SBPs and the imaged. For example, biological samples may be covered with a layer of paraffin wax which is removed through the use of a suitable solvent (e.g. xylene). The solvents employed in the removal of the materials from prior stages in sample processing may also dissolve the reference particles, which can result in both leeching of the reference atoms onto the sample, which contaminates the sample and affects the resulting image, in addition the amount of reference atoms present in the reference particles may be reduced, affecting the accuracy of the absolute normalisation and calibration of the calibrator. Accordingly, in some embodiments, the sample is loaded onto the sample carrier and the materials from prior stages in sample processing removed with a suitable solvent, before sample carrier is contacted with the suspension of at least one reference particle and the reference particles fused to the sample carrier.

In some embodiments of the invention, the reference particles comprise more than one component, for example the reference particles may be core-shell particles comprising a metal core surrounded by a polymer shell. Those skilled in the art will appreciate that such reference particles will need to be heated above the T_{g} of the polymer shell (i.e. and not the melt temperature of the metal core) as this is the material that fuses to the sample carrier. In addition, the T_{g} of such a polymer shell is more amenable for use with the method of the invention than the melt temperature of the metal, for the reasons stated above.

### Kits for Preparing Imaging Mass Calibrators

The invention also provides a series of kits of use in performing methods as disclosed herein. For instance, the kits may comprise a suspension comprising at least one set of reference particles comprising at least one reference atom. The kits may comprise a suspension comprising at least one set of reference particles, wherein the at least one set of reference particles comprises more than one different reference atom, for example at least two, at least three, at least four, at least five, at least six, at least seven, at least eight, or at least ten different reference atoms (i.e. different reference atoms being atoms of differing atomic mass). The kits may also comprise a suspension comprising more than one set of reference particles wherein each set of reference particles comprises a different reference atom, the different reference atom may be present in the same amount in each particle within a set of particles. The kits may also comprise a suspension comprising more than one set of the reference particles described above, for example wherein the suspension comprises more than one set of reference particles and each set of reference particles comprises a different amount of the same reference atom and/or a different referencing atom. The kits may also comprise a suspension comprising at least one set of reference particles wherein each set of reference particles comprises an elemental coding distinct from the other sets of reference particles (the elemental coding being formed of reference atoms, and combinations thereof, as described above). The particle suspensions provided by the invention include those composed of materials suitable for fusion to sample carriers by heating. In some embodiments, the particle suspensions provided by the invention include those composed of materials suitable for fusion to sample carriers by solvent annealing. The suspensions include those with a concentration of particles high enough that they can be used in methods of making an imaging mass calibrator of the invention (e.g. a concentration of particles 1×10⁸ or higher). In some embodiments, the kit of the invention comprises a set of reference particles, suspension of reference particles or calibration series of reference particles and instructions for fusing the reference particles to a sample carrier to make an imaging mass calibrator of the invention. Likewise, in some embodiments, the kit of the invention comprises a set of reference particles, suspension of reference particles or calibration series of reference particles which are for fusing (by heating or solvent annealing) to a sample carrier to make an imaging mass calibrator of the invention.

In some embodiments, the kits comprise a set of reference particles (e.g. a suspension) comprising n reference atoms of each type, where n=10,000,000-30,000,000. A set of reference particles may comprise at least (n×2), at least (n×4), at least (n×8), at least (n×16), or at least (n×32) reference atoms. In some embodiments, a set of reference particles comprise at least (n×3), such as at least (n×9), at least (n×27), or at least (n×81) reference atoms. In some embodiments, a set of reference particles comprising two or more types of reference atom may comprise at least (n/32), such as at least (n/16), at least (n/8), at least (n/4), or at least (n/2) of each type of reference atom. In some embodiments, a set of reference particles comprising two or more types of reference atom may comprise at least (n/81), such as at least (n/27), at least (n/9), or at least (n/3) of each type of reference atom. In some embodiments, the particles discussed in this paragraph have an average diameter of less than 10 μm.

In some embodiments, the kits comprise a set of reference particles e.g. a suspension) comprising more than one type of reference atom, wherein the particle comprises n reference atoms of each type, where n=10,000,000-30,000,000. A set of reference particles may comprise at least (n×10²) of each type of reference atom, such as at least (n×10³), at least (n×10⁴), at least (n×10⁵), at least (n×10⁶), at least (n×10⁷) of each type of reference atom. In some embodiments, sets of reference particles comprise at least (n×10⁻⁵), at least (n×10⁻⁴), at least (n×10⁻³), or at least (n×10⁻²) of each type of reference atom. In some embodiments, the particles discussed in this paragraph have an average diameter of less than 10 μm.

In some embodiments, the kits comprise a set of the reference particles (e.g. a suspension) comprising n×10⁻⁵-n×10⁷ of each type of reference atom, such as n×10⁻⁵-n×10⁶ of each type of reference atom, n×10⁻⁵-n×10⁵ of each type of reference atom, n×10⁻⁵-n×10⁴ of each type of reference atom, n×10⁻⁴-n×10³ of each type of reference atom, n×10⁻³-n×10² of each type of reference atom, or n×10⁻²-n×10¹ of each type of reference atom; where n=10,000,000-30,000,000.

In some embodiments, the kits may comprise a suspension comprising reference particles comprise at least five different types of reference atom, including ¹⁴⁰Ce, ¹⁵¹Eu, ¹⁵³Eu, ¹⁶⁵Ho, ¹⁷⁵Lu, and comprising 15,000,000-20,000,000 ¹⁴⁰Ce reference atoms, for example 17,500,000 to 22,500,000, or 19,000,000-21,000,000 ¹⁴⁰Ce reference atoms; 6,000,000-16,000,000 ¹⁵¹Eu reference atoms, for example 8,500,000-13,500,000, 10,000,000-12,000,000, or about 11,000,000 ¹⁵¹Eu reference atoms; 8,000,000-17,000,000 ¹⁵³Eu reference atoms, for example 9,500,000-14,500,000, 11,000,000-13,000,000, or about 12,000,000 ¹⁵³Eu reference atoms; 2,000,000-12,000,000 ¹⁶⁵Ho reference atoms, for example 4,500,000-9,500,000, 6,000,000-8,000,000, or about 7,000,000 ¹⁶⁵Ho reference atoms; and/or 5,000,000-15,000,000 ¹⁷⁵Lu reference atoms, for example 7,500,000-12,500,000 ¹⁷⁵Lu reference atoms, 9,000,000-11,000,000, or about 10,000,000 ¹⁷⁵Lu reference atoms.

In certain embodiments, the kits may comprise a suspension comprising more than one set of reference particles, wherein each set of reference particles comprises multiple different reference atoms, i.e. a mixture of different reference atoms, with the same amount of each different reference atom in each particle in the set. In addition, the different sets of reference particles in the suspension comprise different amounts of each different reference atom. In said embodiment, the amount of each different reference atom in each set of reference particles is known, thus facilitating construction of calibration curves for multiple mass channels.

### Calibration Series

The kits can comprise sets of reference particles comprising different amounts of reference atoms can therefore be provided in combination to afford a “calibration series” of reference particles. A calibration series can comprise at least 2 sets, such as at least 3, at least 4, at least 5, at least 6, at least 7, at least 8, at least 9, or 10 or more sets of reference particles comprising different amounts of reference atoms.

In some embodiments, the kits of the invention comprise a number of containers, for example at least 2, such as at least 3, at least 4, at least 5, at least 6, at least 7, at least 8, at least 9, or 10 containers, wherein each container comprises a different set of reference particles of a calibration series.

In some embodiments, the kits comprise a calibration series comprising at least three sets of reference particles, having n/m, n and n×m of each type of reference atom; where n=10,000,000-30,000,000, where m=3, 4, 5, 6, 7, 8 or 9 or 20. In some embodiments, the calibration series further comprises one or more of sets comprising n/m⁵ of each type of reference atom, n/m⁴ of each type of reference atom, n/m³ of each type of reference atom, n/m² of each type of reference atom, n× m² of each type of reference atom, n×m³ of each type of reference atom, n×m⁴ of each type of reference atom, n×m⁶ of each type of reference atom and n×m⁶ of each type of reference atom; where n=10,000,000-30,000,000, and wherein m equals the value of m in the n/m, n and n×m series.

In some embodiments, the kits comprise a calibration series comprising at least three sets of reference particles, having n×10⁻¹, n and n×10⁻² of each type of reference atom; where n=10,000,000-30,000,000. In some embodiments, the calibration series further comprises one or more of sets comprising n×10⁻⁵-n×10⁻⁴ of each type of reference atom, n×10⁻⁴-n×10⁻³ of each type of reference atom, n×10⁻³-n×10⁻² of each type of reference atom, n×10⁻²-n×10⁻¹, n×10²-n×10³ of each type of reference atom, n×10³-n×10⁴ of each type of reference atom, n×10⁴-n×10⁵ of each type of reference atom, and n×10⁵-n×10⁶ of each type of reference atom; where n=10,000,000-30,000,000.

In some embodiments, the kits comprise a calibration series comprising at least three sets of reference particles, having n/2, n and n×2 of each type of reference atom; where n=10,000,000-30,000,000. In some embodiments, the calibration series further comprises one or more of sets comprising n/64 of each type of reference atom, n/32 of each type of reference atom, n/16 of each type of reference atom, n/8 of each type of reference atom, n/4 of each type of reference atom, n×4 of each type of reference atom, n×8 of each type of reference atom, n×16 of each type of reference atom, n×32 of each type of reference atom and n×64 of each type of reference atom; where n=10,000,000-30,000,000.

In some embodiments, the kits comprise a calibration series comprising at least five different types of reference atom, for example ¹⁴⁰Ce, ¹⁵¹Eu, ¹⁵³Eu, ¹⁶⁵Ho, ¹⁷⁵Lu, and comprise sets of reference particles comprising between 1,000,000-3,000,000 reference atoms, 3,000,000-5,000,000 reference atoms, 5,000,000-10,000,000 reference atoms, 10,000,000-20,000,000 reference atoms, 20,000,000-40,000,000 reference atoms, 40,000,000-60,000,000 reference atoms, 60,000,000-80,000,000 reference atoms, 80,000,000-100,000,000 reference atoms, 100,000,000-140,000,000 reference atoms, and/or 140,000,000-200,000,000 reference atoms in total. For example, a calibration series may comprise sets of reference particles comprising about 2,000,000 reference atoms, about 4,000,000 reference atoms, about 7,500,000 reference atoms, about 15,000,000 reference atoms, about 30,000,000 reference atoms, about 50,000,000 reference atoms, about 70,000,000 reference atoms, about 90,000,000 reference atoms, about 120,000,000 reference atoms, and/or about 160,000,000 reference atoms in total.

In some embodiments, the kits comprise a calibration series comprising at least five different types of reference atom, for example ¹⁴⁰Ce, ¹⁵¹Eu, ¹⁵³Eu, ¹⁶⁵Ho, ¹⁷⁵Lu, and comprise sets of reference particles comprising between 250,000-750,000, 750,000-1,250,000, 1,250,000-3,000,000, 3,000,000-5,000,000, 5,000,000-9,000,000, 9,000,000-14,000,000, 14,000,000-18,000,000, 18,000,000-22,000,000, 22,000,000-34,000,000, and/or 34,000,000-44,000,000 of each reference atom. For example, a calibration series may comprise sets of reference particles comprising about 500,000, about 1,000,000, about 2,000,000, about 4,000,000, about 7,000,000, about 12,000,000, about 16,000,000, about 20,000,000, about 28,000,000, and/or about 39,000,000 of each reference atom.

In some embodiments, the kits comprise a calibration series comprising sets of reference particles having between 300,000-1,000,000 ¹⁴⁰Ce reference atoms, 1,000,000-1,500,000 ¹⁴⁰Ce reference atoms, 1,500,000-3,500,000 ¹⁴⁰Ce reference atoms, 3,500,000-7,000,000 ¹⁴⁰Ce reference atoms, 7,000,000-11,000,000 ¹⁴⁰Ce reference atoms, 11,000,000-19,000,000 ¹⁴⁰Ce reference atoms, 19,000,000-23,000,000 ¹⁴⁰Ce reference atoms, 23,000,000-30,000,000 ¹⁴⁰Ce reference atoms, 30,000,000-44,000,000 ¹⁴⁰Ce reference atoms, and/or 44,000,000-56,000,000 ¹⁴⁰Ce reference atoms. In some embodiments a calibration series comprises sets of reference particles having about 700,000, about 1,300,000, about 2,500,000, about 5,500,000, about 9,000,000, about 16,000,000, about 21,000,000, about 26,000,000, about 37,000,000, and/or about 51,000,000 ¹⁴⁰Ce reference atoms.

In some embodiments, the kits comprise a calibration series comprising sets of reference particles having between 200,000-500,000 ¹⁵¹Eu reference atoms, 500,000-1,000,000 ¹⁵¹Eu reference atoms, 1,000,000-2,000,000 ¹⁵¹Eu reference atoms, 2,000,000-4,000,000 ¹⁵¹Eu reference atoms, 4,000,000-6,000,000 ¹⁵¹Eu reference atoms, 6,000,000-11,000,000 ¹⁵¹Eu reference atoms, 11,000,000-13,000,000 ¹⁵¹Eu reference atoms, 13,000,000-18,000,000 ¹⁵¹Eu reference atoms, 18,000,000-23,000,000 ¹⁵¹Eu reference atoms, and/or 23,000,000-37,000,000 ¹⁵¹Eu reference atoms. In some embodiments a calibration series comprises sets of reference particles having about 400,000, about 750,000, about 1,500,000, about 3,000,000, about 5,000,000, about 9,000,000, about 12,000,000, about 15,000,000, about 21,000,000, and/or about 29,000,000 ¹⁵¹Eu reference atoms.

In some embodiments, the kits comprise a calibration series comprising sets of reference particles having between 200,000-600,000 ¹⁵³Eu reference atoms, 600,000-1,000,000 ¹⁵³Eu reference atoms, 1,000,000-2,000,000 ¹⁵³Eu reference atoms, 2,000,000-4,000,000 ¹⁵³Eu reference atoms, 4,000,000-7,000,000 ¹⁵³Eu reference atoms, 7,000,000-11,000,000 ¹⁵³Eu reference atoms, 11,000,000-14,000,000 ¹⁵³Eu reference atoms, 14,000,000-18,000,000 ¹⁵³Eu reference atoms, 18,000,000-26,000,000 ¹⁵³Eu reference atoms, and/or 26,000,000-36,000,000 ¹⁵³Eu reference atoms. In some embodiments, a calibration series comprises sets of reference particles having about 400,000, about 800,000, about 1,600,000, about 3,000,000, about 5,500,000, about 10,000,000, about 13,000,000, about 16,000,000, about 22,000,000, and/or about 31,000,000 ¹⁵³Eu reference atoms.

In some embodiments, the kits comprise a calibration series comprising sets of reference particles having between 200-000-300,000, ¹⁶⁵Ho reference atoms, 300-000-700,000 ¹⁶⁵Ho reference atoms, 700-000-1,300,000 ¹⁶⁵Ho reference atoms, 1,300,000-2,800,000 ¹⁶⁵Ho reference atoms, 2,800,000-3,500,000 ¹⁶⁵Ho reference atoms, 3,500,000-7,500,000 ¹⁶⁵Ho reference atoms, 7,500,000-9,000,000 ¹⁶⁵Ho reference atoms, 9,000,000-11,000,000 ¹⁶⁵Ho reference atoms, 11,000,000-17,000,000 ¹⁶⁵Ho reference atoms, and/or 17,000,000-23,000,000 ¹⁶⁵Ho reference atoms. In some embodiments, a calibration series comprises sets of reference particles having about 250,000, about 500,000, about 1,000,000, about 2,000,000, about 3,500,000, about 6,000,000, about 8,000,000, about 10,000,000, about 14,000,000, and/or about 20,000,000 ¹⁶⁵Ho reference atoms.

In some embodiments, the kits comprise a calibration series comprising sets of reference particles having between 200,000-400,000 ¹⁷⁵Lu reference atoms; 400,000-1,000,000 ¹⁷⁵Lu reference atoms; 1,000,000-1,500,000 ¹⁷⁵Lu reference atoms; 1,500,000-3,500,000 ¹⁷⁵Lu reference atoms; 3,500,000-5,500,000 ¹⁷⁵Lu reference atoms; 5,500,000-9,000,000 ¹⁷⁵Lu reference atoms; 9,000,000-11,000,000 ¹⁷⁵Lu reference atoms; 11,000,000-15,000,000 ¹⁷⁵Lu reference atoms; 15,000,000-21,000,000 ¹⁷⁵Lu reference atoms; and/or 21,000,000-31,000,000 ¹⁷⁵Lu reference atoms. In some embodiments, a calibration series comprises sets of reference particles having about 300,000, about 700,000, about 1,300,000, about 2,500,000, about 4,500,000, about 8,000,000, about 10,500,000, about 13,000,000, about 19,000,000, and/or about 26,000,000 ¹⁷⁵Lu reference atoms.

### Particle Diameter

In some embodiments, the kits of the invention comprise a suspension (e.g. more than one suspension) of beads of a diameter of between 1 μm to 50 μm, for example between 1 μm to 40 μm, 1 μm to 30 μm, 1 μm to 20 μm, or 1 μm to 10 μm, or between 1 μm to 5 μm.

In some embodiments, the kits comprise reference particles with a diameter between 1 μm to 50 μm and between n×10⁻⁵-n×10⁵ reference atoms in total, such as a diameter between 1 μm to 40 μm and between n×10⁻⁴-n×10⁴ reference atoms in total, a dimeter between 1 μm to 30 μm and between n×10⁻³-n×10³ reference atoms in total, a dimeter between 1 μm to 20 μm and between n×10⁻²-n×10² reference atoms in total, a diameter between 1 μm to 10 μm and between n×10¹-n×10¹ reference atoms in total, or a diameter between 1 μm to 5 μm and between n×10⁻¹-n×10¹ reference atoms in total; where n=10,000,000-30,000,000.

In some embodiments, the kits comprise reference particles comprising at least five different types of reference atom, for example ¹⁴⁰Ce, ¹⁵¹Eu, ¹⁵³Eu, ¹⁶⁵Ho, ¹⁷⁵Lu, and have a diameter between 1 μm to 50 μm and between 1,000-300,000,000 reference atoms in total, for example a diameter between 1 μm to 40 μm and between 2,000-200,000,000 reference atoms in total, a dimeter between 1 μm to 30 μm and between 100,000-125,000,000 reference atoms in total, a dimeter between 1 μm to 20 μm and between 1,000,000-100,000,000 reference atoms in total, a diameter between 1 μm to 10 μm and between 30,000,000-90,000,000 reference atoms in total, or a diameter between 1 μm to 5 μm and between 50,000,000-70,000,000 reference atoms in total. In some embodiments, the particles are about 3 μm in diameter and comprise about 60,000,000 reference atoms in total.

In some embodiments, the kits comprise suspensions comprising reference particles comprising at least five different types of reference atom, for example ¹⁴⁰Ce, ¹⁵¹Eu, ¹⁵³Eu, ¹⁶⁵Ho, ¹⁷⁵Lu, and comprise reference particles having a diameter of 1 μm to 50 μm and between 1,000-100,000,000 of each type of reference atom, for example a diameter between 1 μm to 40 μm and between 5,000-50,000,000 of each type of reference atom, a dimeter between 1 μm to 30 μm and between 100,000-30,000,000 of each type of reference atom, a dimeter between 1 μm to 20 μm and between 200,000-20,000,000 of each type of reference atom, a diameter between 1 μm to 10 μm and between 1,000,000-20,000,000 of each type of reference atom, or a diameter between 1 μm to 5 μm and between 10,000,000-20,000,000 of each type of reference atom. In some embodiments the kits comprising suspensions comprise reference particles about 3 μm in diameter and that comprise about 15,000,000 of each type of reference atom.

### Suspension Concentrations

In some embodiments, the kits of the invention comprise a suspension (e.g. more than one suspension) of reference particles at a concentration between 1×10⁶ to 1×10¹⁵ particles per ml, for example from 1×10⁷ to 1×10¹³ particles per ml, 1×10⁸ to 1×10¹³ particles per ml, 1×10⁹ to 1×10¹² particles per ml, 1×10⁹ to 1×10¹¹ particles per ml, or about 1×10¹⁰ particles per ml. In some embodiments, the kits of the invention comprise a suspension of the beads at between 1-50% solids content, for example between 10-40%, 10-30%, 15-25%, 15-20%, or about 18% solids content. Concentrated suspensions of the beads (e.g. above 1×10⁹ particles per ml or 10% solids content), typically require diluting by the user in order to obtain concentrations of the particles such that when the suspension is pipetted onto the sample carrier, an adequate distribution of the particles on the sample carrier is achieved. It will be recognised that such concentrated suspensions are convenient as the kits can be supplied to the user without the need to transport large volumes of solvent.

In some embodiments, the kits of the invention comprise a suspension (e.g. more than one suspension) of reference particles at a concentration for use in the methods of the invention, for example between 1×10⁶ to 1×10¹⁵ particles per ml, for example from 1×10⁷ to 1×10¹³ particles per ml, 1×10⁷ to 1×10¹² particles per ml, 1×10⁷ to 1×10¹⁰ particles per ml, 1×10⁷ to 1×10⁹ particles per ml, or about 1×10⁸ particles per ml.

In some embodiments, the kits of the invention comprise a suspension (e.g. more than one suspension) of reference particles having a diameter between 1 μm to 50 μm wherein the particle concentration is 1×10⁶ to 1×10¹⁵ particles per ml, having a diameter between 1 μm to 40 μm wherein the particle concentration is 1×10⁷ to 1×10¹³ particles per ml, having a diameter between 1 μm to 30 μm wherein the particle concentration is 1×10⁸ to 1×10¹³ particles per ml, having a diameter between 1 μm to 20 μm wherein the particle concentration is 1×10⁹ to 1×10¹² particles per ml, having a diameter between 1 μm to 10 μm wherein the particle concentration is 1×10⁹ to 1×10¹¹ particles per ml, having a diameter between 1 μm to 5 μm wherein the particle concentration is 1×10⁹ to 1×10¹⁰ particles per ml, or having a diameter between about 3 μm wherein the particle concentration is about 1×10¹⁰ particles per ml.

In some embodiments, the kits of the invention comprise a suspension (e.g. more than one suspension) of reference particles at a concentration between 1×10⁶ to 1×10¹⁵ particles per ml and have between n×10⁻⁵-n×10⁷ of each type of reference atom, for example between 1×10⁷ to 1×10¹³ particles per ml and n×10⁻⁵-n×10⁶ of each type of reference atom, between 1×10⁸ to 1×10¹³ particles per ml and n×10⁻⁵-n×10⁵ of each type of reference atom, between 1×10⁹ to 1×10¹² particles per ml and n×10⁻⁴-n×10⁴ of each type of reference atom, between 1×10⁹ to 1×10¹⁰ particles per ml and n×10⁻³-n×10³ of each type of reference atom, between 1×10⁹ to 1×10¹⁰ particles per ml and n×10⁻²-n×10² of each type of reference atom, between 1×10⁹ to 1×10¹⁰ particles per ml and n×10⁻¹-n×10¹ of each type of reference atom; where n=10,000,000-30,000,000.

In some embodiments, the kits of the invention comprise a suspension of reference particles comprising at least five different types of reference atom, for example ¹⁴⁰Ce, ¹⁵¹Eu, ¹⁵³Eu, ¹⁶⁵Ho, ¹⁷⁵Lu, at a concentration between 1×10⁶ to 1×10¹⁵ particles per ml wherein the beads comprise between 1,000-100,000,000 of each type of reference atom, for example a concentration between 1×10⁷ to 1×10¹³ particles per ml and between 5,000-50,000,000 of each type of reference atom, a concentration between 1×10⁸ to 1×10¹³ particles per ml and between 100,000-30,000,000 of each type of reference atom, a concentration between 1×10⁹ to 1×10¹² particles per ml and between 200,000-25,000,000 of each type of reference atom, a concentration between 1×10⁹ to 1×10¹¹ particles per ml and between 1,000,000-25,000,000 of each type of reference atom, or a concentration between 1×10⁹ to 1×10¹⁰ particles per ml and between 10,000,000-25,000,000 of each type of reference atom.

In some embodiments, the kits of the invention comprise a pipetting tool, configured to pipette multiple suspensions/solutions onto discrete areas of the sample carrier. For example, in some embodiments the kits comprise pipettes configured to pipette at least 2, at least 3, at least 4, at least 5, at least 6, at least 7, at least 8, or at least 10 suspensions onto discrete areas of the sample carrier. The pipettes may be configured to pipette about 1 μm, about 2 μm, about 3 μm, about 4 μm, about 5 μm, about 8 μm, about 10 μm, about 15 μm, or about 20 μm of suspension onto the slides. The pipetting tool may be configured to pipette multiple suspensions onto the sample carrier in neighbouring areas of the sample carrier. The pipetting tool may be configured to pipette suspensions/solutions onto discrete 1 mm×1 mm, 2 mm×2 mm, 4 mm×4 mm, 6 mm×6 mm, 8 mm×8 mm, 10 mm×10 mm or 10 mm×20 mm areas of the sample carrier.

Alternatively, instead of pipetting, the particles may be transferred by a pin replicator, to pattern the particles onto the sample carrier in a regular array pattern. Accordingly, in some instances the kit may comprise a replicator.

In some embodiments, kits of the invention comprise a reference particle comprising a reference atom, and a mass-tagging reagent comprising a labelling atom, wherein the reference atom is the same as the labelling atom. In some embodiments, kits comprise reference particles that contains at least two, e.g. at least 3, at least 4, at least 5, at least 10, at least 20 or at least 50 different reference atoms, together with at least two, e.g. at least 3, at least 4, at least 5, at least 10, at least 20 or at least 50 mass-tagging reagents comprising a labelling atom, respectively, wherein each reference atom is a labelling atom in a mass tagging reagent.

### Sampling, Normalisation and Calibration

The present invention provides a method of monitoring the performance of an mass imaging apparatus (imaging mass cytometer/imaging mass spectrometer), comprising the steps of providing an imaging mass calibrator comprising a sample carrier with at least one reference particle fused thereto, wherein the at least one fused reference particle comprises at least one reference atom, determining an average integral signal intensity per fused reference particle, and monitoring the average integral signal intensity per fused reference particle. In other words, by detecting the elemental ions present in reference particles fused to the surface of the sample carrier at certain points during the imaging of a sample, or between samples, the methods of the present invention facilitate the normalisation of the signal intensity detected when imaging a sample and can thus observe and account for drift in instrument sensitivity that occurs during the imaging of the sample. Typically multiple fused reference particles are on the sample carrier, to facilitate monitoring of the average integral signal intensity per fused reference particle over time. As detailed below, where different samples are on sample carriers to which the same reference particles have been fused, performance can be monitored across multiple samples, and even across different apparatus.

Sampling can be achieved by the ablation of material from the sample carrier using the procedure typically applied in imaging mass cytometry.

In general terms, the sample on imaging mass calibrator is placed in a sample chamber, which is the component of the mass imaging apparatus in which the sample is placed when it is subjected to analysis. The sample chamber comprises a stage, which holds the mass imaging calibrator, and so, in operation, the sample. The sampling and ionisation system acts to remove material from the sample in the sample chamber which is converted into ions, either as part of the process that causes the removal of the material from the sample or via a separate ionisation system downstream of the sampling system. The different types of apparatus are discussed in more detail below.

The ionised material is then analysed by the second system which is the detector system. The detector system can take different forms depending upon the particular characteristic of the ionised sample material being determined, for example a mass detector in mass spectrometry-based apparatus.

Thus, in operation, the sample is taken into the apparatus, is sampled to generate ionised material using a laser system (sampling may generate vaporous/particular material, which is subsequently ionised by the ionisation system), and the ions of the sample material are passed into the detector system. Although the detector system can detect many ions, most of these will be ions of the atoms that naturally make up the sample. By labelling the sample with atoms not present in the material being analysed under normal conditions, or at least not present in significant amounts (for example certain transition metal atoms, such as rare earth metals; see section on labelling below for further detail), specific characteristics of the sample can be determined.

### Sampling the Fused Reference Particles

The average integral signal intensity per fused reference particle is determined in the method of the present invention by sampling at least one whole fused reference particle, and determining the integral signal intensity associated with said reference particle(s). The integral signal intensity is the signal intensity of the whole particle. Accordingly, this may be achieved by sampling the whole particles from the sample carrier in one event (e.g., when laser spot size is >the diameter of the fused particle), or in multiple events (e.g. where the laser spot size is smaller than the diameter, and multiple laser shots are required to ablate all of the reference particle) and then summing the signals resulting from the individual shots to generate the integral signal from the whole particle.

Thus, sampling may include ablating discrete reference particles by laser ablation. In certain embodiments, ionizing and atomizing reference particles may be performed inductively coupled plasma. In other embodiments, ionizing and atomizing reference particles may include laser desorption ionisation to form sample ions (as will be appreciated by one of skill in the art, the apparatus required for his can be based on sampling and ionization systems as described herein.

The average integral signal intensity per fused reference particle may be determined by sampling at least one, at least two, at least three, at least four, at least five, at least six, at least seven, at least eight, at least ten, or at least twenty reference particles; and calculating an average integral intensity thereof. For instance, in some embodiments, three reference particles are sampled in generating the average integral signal intensity per fused reference particle. In some embodiments, five reference particles are sampled in generating the average integral signal intensity per fused reference particle.

Accordingly, in some embodiments of the invention, identification of individual reference particles is performed, such that individual reference particles may be ablated. Alternatively, it will be apparent to those skilled in the art that should the reference particles agglomerate on the sample carrier, provided the total number of reference particles can be identified, ablation and sampling of all the agglomerated reference particle can be performed to obtain an integral signal intensity for all reference particles present in the agglomeration, such that an average integral signal intensity per fused reference particle can still be calculated.

Those skilled in the art will appreciate that the reference particles will be of a certain size to allow both the incorporation of a sufficient amount of reference atoms to ensure adequate signal detection, however the particles should not be of such a size that following fusion to the sample carrier, the particle spreads to such an extent that the time required to ablate the whole particle from the sample carrier becomes impractical. E.g. for ablation of a 2 μm diameter reference particle with a laser having a spot size of 1 μm will require 4 laser shots to ablate the whole particle and so determine the integral signal intensity. With greater fused particle diameter, more and more shots with a laser having a spot size of 1 μm will be required, which imposed a time cost. Thus, those skilled in the art will appreciate that fused reference particles in the range of 1 μm to 50 μm, including 1 μm to 40 μm, 1 μm to 30 μm, 1 μm to 20 μm, or 1 μm to 10 μm following fusion are suitable for use in the present invention.

In certain embodiments, a camera is used to identity the reference particle for sampling. Accordingly, the reference particle may be individually resolvable if the individual reference particles can be identified (e.g., by light microscopy or elemental imaging) with a high degree of confidence, such as 90%, 80% or 70% confidence that the reference particle is isolated. Thus, elemental ions from individual reference particles may be detected without simultaneously detecting elemental ions from another reference particle. Accordingly, in some embodiments the method comprises the steps of using a camera to identify individual particles fused to the sample carrier, prior to sampling the individual particle to determine an integral signal intensity.

As discussed on page 42, reference particles may be individually detected by distributing said reference particles across the sample carrier at a low enough density such that the reference particles can be ablated and sampled individually such that elemental ions from multiple reference particles are rarely sampled and detected simultaneously. The method for depositing the reference particles may also reduce aggregation of reference particles, as described herein. In certain embodiments, less than 50%, less than 40%, less than 30%, less than 20%, less than 10%, or less than 5% of the elemental ions detected from reference particles are detected simultaneously with another reference particle.

In certain embodiments, reference particles may each contain coding atoms that can be used to distinguish reference particles based on their elemental composition. Elemental ions pertaining to 2 or more codes that are detected in the same location would indicate the presence of 2 or more different reference particles. The elemental ions detected in the sampling of that location would therefore not be used for normalization. Coding atoms in a reference particle may indicate additional properties of the reference particle, such as the number of certain elements or isotopes in the reference particle, as noted above.

In embodiments where the reference particle is smaller than the sampling spot size (e.g., a laser ablation spot size or a primary beam spot size), the reference particle elemental ions that are detected in close spatial proximity (such as in neighbouring pixels) may not be used in normalization as they may originate from two or more reference particles.

### Normalizing Signal Intensity

Mass imaging apparatus (imaging mass cytometers and imaging mass spectrometers) suffer from instrument drift, whereby the factors including ion optics drift, surface charging, detector drift (e.g., aging), temperature and gas flows drifts affecting diffusion, and electronics behaviour (e.g., plasma power, ion optics voltages, etc) can cause instrument sensitivity drift both during the imaging of a single sample and between imaging different samples. Thus, normalizing signal intensity is desirable as it allows for a consistent image to be obtained, in addition allows the comparison of images of different samples. Recording the signal intensity detected from sampling reference particles before, during and/or after imaging of a sample, or samples, using the imaging mass calibrator and methods of the invention ensures that any drift in the instrument sensitivity does not affect the resulting image. Accordingly, when the imaging mass calibrator of the present invention further comprises a sample, the method of the invention may further comprise normalizing the signal detected during the imaging of the sample using the calibrator of the invention. In addition, the method may further comprise normalizing the signal detected between different samples using the calibrator of the invention.

The imaging mass calibrator and methods of the invention can be used to normalise signal intensity detected during mass imaging (e.g. imaging mass cytometry). Thus, an initial average integral signal intensity can be determined for a set of reference particles (i.e. before imaging the sample). Subsequently, different reference particles in the same set of reference particles can be sampled again after a period of time has passed and an average integral signal intensity per reference particle determined at this point (i.e. during and after the imaging of the sample). According to the invention, an average integral intensity per reference particle can be calculated at t=0 (t=time). Subsequently, an average integral signal intensity per fused reference particle can be calculated at t=nx (x=sampling interval; n=integer), for user defined periods as easily determined by the skilled person based on the imaging being performed, for example the total time required to image the sample.

The average integral signal intensities per reference particle can be compared from different time points, for example before imaging a sample and at various time points throughout the imaging of the sample. For example, in the method of normalisation of the invention, at least one reference particle is sampled at least every 10 minutes (i.e x=10), at least every 20 minutes (i.e x=20), at least every 30 minutes (i.e x=30), at least every 40 minutes (i.e x=40), at least every 50 minutes (i.e x=50), at least every 60 minutes (i.e x=60), at least every 90 minutes (i.e x=90), at least every 120 minutes (i.e x=120), or at least every 300 minutes (i.e x=300). The number of times the reference particles can be sampled (i.e n in the above equation) can be varied depending on the time required to image the sample. For example, reference particles can be sampled over a period of at least 5 hours, at least 10 hours, at least 15 hours, at least 20 hours, at least 24 hours, at least 48 hours, at least 72 hours, or at least 96 hours.

Comparison of the average integral signal intensities per reference particle at t=0 and each of the t=nx series is then performed in the method of normalisation of the present invention. If a variation in average integral signal intensity is detected, the signal intensity associated with the mass tags on the sample can therefore be adjusted.

According to the invention, normalisation of the signal intensity detected during imaging at t=nx can be achieved by using the following equation:

$$Normalised Sample Signal Intensity at {t}_{n x} = S ample Signal Intensity at {t}_{n x} \times \frac{\begin{matrix}Average Integral Signal Intensity per \\ Fused Reference Particle at {t}_{n x}\end{matrix} }{\begin{matrix}Average Integral Signal Intensity per \\ Fused Reference Particle at {t}_{0}\end{matrix} }$$

The method of the fusing reference particles of the invention also enables a method by which a standard of known elemental composition and amount can be fused onto multiple samples. Thus, the method of normalization of the invention can also be applied across multiple samples.

The average integral signal intensity can be compared and normalized absolutely.

The variation in the integral average signal intensity may be expressed as a percentage of the absolute initial average integral signal intensity. Those skilled in the art will recognize that such an absolute technique will facilitate comparison and thus normalization of the signal intensities detected for different samples, including samples imaged on different days.

Accordingly, the present invention provides a method of normalizing the signal intensity detected in an imaging mass cytometer during the imaging of a sample. In some embodiments the method therefore comprises preparing an imaging mass calibrator comprising a sample carrier comprising a sample and at least two fused reference particles, sampling at least one fused reference particle, determining an average integral signal intensity per fused particle for the at least one fused reference particle, imaging a portion of the sample, sampling at least one fused reference particle, and imaging a further portion of the sample. The steps of sampling at least one fused reference particle and imaging a further portion of the sample may be continued until the whole sample is imaged. For instance, reference particles may be sampled at least two times during the imaging of the sample, such as at least three times, at least four times, at least five times, at least ten times or more than ten times. The signal intensity detected during imaging of the sample is normalized using the above equation.

In addition, the present invention provides a method of normalizing the signal intensity detected in an imaging mass cytometer when imaging different samples, including those imaged on different days. Accordingly, the invention provides a method of imaging multiple samples using mass imaging apparatus (e.g. imaging mass cytometer), comprising the steps of: (i) providing a first imaging mass calibrator comprising a sample carrier comprising a first sample and at least one fused reference particle, (ii) sampling at least one fused reference particle on the first imaging mass calibrator, (iii) determining an average integral signal intensity per fused particle for the at least one fused reference particle, (iv) imaging the first sample, (v) providing a second imaging mass calibrator comprising a second sample and the same at least one fused reference particle as the first calibrator, (vi) sampling the at least one fused reference particle on the second imaging mass calibrator, (vii) determining an average integral signal intensity per fused particle for the at least one fused particle, (viii) imaging the second sample, (ix) comparing the absolute intensities of the average integral signal intensities per fused particle detected for the fused particles on the first and second calibrators, and (x) normalizing the signal intensity detected during imaging of the second sample using the equation listed above. The steps of sampling at least one fused reference particle and imaging a further portion of each sample may be continued until the whole sample is imaged (e.g. repeating steps (ii)-(iv) and/or steps (vi)-(viii)). For instance, reference particles may be sampled at least two times during the imaging of a sample, such as at least three times, at least four times, at least five times, at least ten times or more than ten times. In some embodiments, the method further comprises imaging at least a third, for example, at least a fourth, fifth, sixth, seventh, eighth, ninth, tenth, 20th, 30th, 40th or 50th sample, comprising repeating steps (v)-(x) the respective number of times for imaging and normalisation of further samples.

Normalisation of the detector may be performed for a specific mass channel, for example a lanthanide, such as Cerium, Europium, Holmium, and/or Lutetium, or for any of the reference atoms discussed on page 15.

In some embodiments of the invention, normalizing the signal intensity during the imaging of a sample comprises normalizing the signal intensity detected for a specific mass channel relative to the mass channel closest in atomic mass to the at least one labelling atom (i.e. the specific mass channel) in the mass tag being sampled and ionized from the sample. For example, the signal intensity can be normalized for the same mass channel as the mass channel being sampled and ionized from the mass tags on the sample. In some embodiments, the signal intensity detected for a specific mass channel is normalized relative to a mass channel within 10 atomic units, within 20 atomic units, within 30 atomic units, within 40 atomic units, within 50 atomic units, or within 60 atomic units, of the atomic mass of the at least one labelling atom being sampled in the mass tag present on the sample.

In some embodiments of the invention, normalizing the signal intensity during the imaging of a sample comprises normalizing the mass channel closest in intensity to the at least one labelling atom in the mass tag that is being sampled and ionized from the sample. For example, the signal intensity can be normalized for a mass channel with substantially the same intensity as the mass channel being sampled and ionized from the at least one labelling atom in the mass tags on the sample.

### Calibration

Accordingly, in some embodiments, the method of the invention comprises providing an imaging mass calibrator comprising at least one fused reference particle, sampling at least one fused reference particle, determining an average integral signal intensity per fused particle for the at least one particle, and calibrating the average integral signal intensity to the known amount of reference atom present in the at least one reference particle and corresponding to the mass channel of the detector being calibrated.

Those skilled in the art will appreciate that care is required when subjecting the area of the sample carrier comprising the fused reference particle to the same treatment steps used to place the sample onto the sample carrier. As stated above, such treatment steps can remove reference atoms from the fused reference particles. However, if the specific treatment steps used for the reference particles contaminate the sample, this may result in the leeching of reference atoms from the fused reference particles onto the sample, thus affecting the image of the sample obtained. For example, when preparing the sample carrier, should both the area comprising the sample and the area comprising the fused reference particle be treated with the same wash solution in the same wash step, then some of solution used to wash the reference particles may contact the sample and vice versa, resulting in the contamination of the sample. Thus, those skilled in the art will appreciate that in order to assess whether variation in sample preparation is affecting the signal intensity detected will required both the sample and the fused reference particles to be subjected to the same treatment steps, they will also appreciate that said treatment steps should ideally be conducted in isolation such that contamination of the sample does not occur.

### Calibration Curve Generation

As noted above, elemental analysis, including elemental mass spectrometry such as mass cytometry and imaging mass cytometry, because of its extreme selectivity and sensitivity, has become a powerful tool for the quantitation of a broad range of bioanalytes including pharmaceuticals, metabolites, peptides and proteins. However the signal generated by the compound can vary between runs due to differences in sample introduction, ionization process, ion acceleration, ion separation, and ion detection.

In some embodiments, to enable quantitation of the analytes in the sample, the measurement from the sample is compared to known standards. Standards can be used to form a calibration curve for the ion counts of reference atoms, and from that absolute quantitation of an analyte can be calculated. In some embodiments, the calibration curve comprises at least 2 points, such as at least 3, at least 4, at least 5, at least 6, at least 7, at least 8, at least 9, or 10 or more points. Typically, at least 3 repeats of each point are performed.

In some embodiments of the invention, the sample carrier may comprise more than one fused reference particle, for example the sample carrier may comprise two, three, four, five, six, seven, or eight sets of at least one fused reference particle wherein each set of at least one fused reference particle comprises a different amount of the same reference atom. The method of the calibrating an imaging mass cytometer of the invention may therefore comprise the steps of sampling each set of at least one fused reference particle, determining an average integral signal intensity per fused reference particle for each set and plotting a calibration curve. In some embodiments of the invention, the sample carrier comprises more than one discrete area, for example wherein the sample carrier comprises at least two, at least three, at least four, at least five, at least six, at least seven or at least eight discrete areas and wherein the fused reference particles in each discrete area comprise a different amount of the same at least one reference atom. In other words, a calibration curve is generated correlating the known level with the ion count.

The calibration curve may be determined prior to analysis of the sample, during the analysis of the sample, or after analysis of the sample. In some instances, calibration may be performed before and after, before and during, during and after or before, during and after the analysis of the sample.

In some embodiments, each different mass channel is calibrated with a different particle (i.e. each set of reference particles comprises a different reference atom). In some embodiments, each reference particle comprises more than one reference atom, such that calibration curves can be generated for multiple mass channels at the same time, to maximise procedural efficiency. In some instances, all mass channels in an experiment can be obtained from the same set of doped beads.

Upon generation of a calibration, the amount of analyte present in a sample can be quantified through comparison of the signal intensity detected for the reference particle analyte with the calibration curve plotted for the reference particle mass channel corresponding to the analyte. As noted above, the integral signal associated with sampling a known number of particular reference atoms may be different from that associated with sampling the same number of a different reference atom (for instances because of differences in the behaviours of the reference atoms e.g. ionisation efficiency). The amount of the reference atoms present in the fused reference particles can therefore be selected to provide a substantially consistent signal intensity for each reference atom detected for each mass channel that is being normalised/calibrated. That is to say, if the same absolute number of atoms of a first reference atom provides a lower signal at the detector than a second reference atom, then a greater absolute quantity should be provided in the reference particle. Accordingly, in some embodiments, when different reference atoms are present in a reference particle, a comparable signal is detected for each of the different reference atoms when the reference particle is sampled.

### Applications of the Quantitation Methods of the Invention

The invention can quantify one or more analytes of interest even from samples and mixtures containing a high number of other biomolecules. As such the invention is particularly useful in quantitation of analytes from biological sample which typically contain a multitude of other species; such as the validation or quantitation of biomarkers from biological samples.

As used herein “biomarker” refers to a protein or polypeptide which is differentially present in samples from subjects having a genotype or phenotype of interest and/or who have been exposed to a condition of interest, as compared to equivalent samples from control subjects not having said genotype or phenotype and/or not having been exposed to said condition.

Particularly relevant phenotypes may be pathological conditions in patients, such as, e.g., cancer, an inflammatory disease, autoimmune disease, metabolic disease, CNS disease, ocular disease, cardiac disease, pulmonary disease, hepatic disease, gastrointestinal disease, neurodegenerative disease, genetic disease, infectious disease or viral infection; vis-à-vis the absence thereof in healthy controls. Other comparisons may be envisaged between samples from, e.g., stressed vs. non-stressed conditions/subjects, drug-treated vs. non drug-treated conditions/subjects, benign vs. malignant diseases, adherent vs. non-adherent conditions, infected vs. uninfected conditions/subjects, transformed vs. untransformed cells or tissues, different stages of development, conditions of overexpression vs. normal expression of one or more genes, conditions of silencing or knock-out vs. normal expression of one or more genes, and so on.

For example, a protein may be denoted as differentially present between two samples or between two sample groups if the protein's quantity in one sample or one sample group is at least about 1.2-fold, at least about 1.3-fold, at least about 1.5-fold, at least about 1.8-fold, at least about 2-fold, at least about 3-fold, at least about 5-fold, at least about 7-fold, at least about 9-fold or at least about 10-fold of its quantity in the other sample or the other sample group; or if the protein is detectable in one sample or one sample group but not detectable in the other sample or the other sample group.

Accordingly, the invention provides a method of diagnosing a condition or disease in a subject, comprising the steps of:

a. providing a sample, on an imaging mass calibrator of the invention, obtained from the subject, wherein the sample has been labelled with one or more mass-tagged SBPs specific for one or more biomarkers of the disease;
b. performing mass cytometry on the sample to determine the level of the one or more labelling atoms in the one or more mass tag;
c. comparing the level of the one or more labelling atoms with the level determined from a healthy control,
wherein a difference in the level of one or more labelling atoms between the subject and control indicates that the subject is suffering from the disease or condition. In some embodiments, the method further comprises sampling a fused particle on the imaging mass calibrator to normalise and/or calibrate the level of the one or more labelling atoms. In some instances, the disease state is indicated by an increase in the level of biomarker vis-à-vis the healthy control. In some instances, the disease state is indicated by a decrease in the level of biomarker vis-à-vis the healthy control. As a person of skill in the art will appreciate, the specific difference will vary (increase or decrease) and will depend both on the biomarker and the disease. In some instances, a conclusion will be drawn on the basis of the relative levels of at least 3, such as at least 5 biomarkers.

Accordingly, the invention provides a method of predicting the likelihood that a treatment for a disease will be successful in a subject, comprising the steps of:

a. providing a sample, on an imaging mass calibrator of the invention, obtained from the subject, wherein the sample has been labelled with one or more mass-tagged SBP specific for one or more biomarkers of the disease;
b. performing mass cytometry on the sample to determine the level of the one or more labelling atoms in the mass tag;
c. comparing the level of the one or more labelling atoms with the level determined from a treatment responsive control,
wherein a difference in the level of one or more labelling atoms between the subject and control indicates that the subject is unlikely to respond to the treatment. In some embodiments, the method further comprises sampling a fused particle on the imaging mass calibrator to normalise and/or calibrate the level of the one or more labelling atoms. In some instances, the level between the sample and the control can differ, with the control level setting an upper or lower limit which is used to determine the likelihood of an effective treatment. For instance, in some embodiments, the sample may be deemed to indicate that the treatment would be effective in the subject if the level of a biomarker is the same as or below the level of the responsive control. In some embodiments, the sample may be deemed to indicate that the treatment would be effective in the subject if the level of a biomarker is the same as or above the level of the responsive control. In some instances, a conclusion will be drawn on the basis of the relative levels of at least 3, such as at least 5 biomarkers.

The invention also provides a method of determining the efficacy of therapy in the treatment of a disease or condition in a subject, comprising the steps of:

a. providing a sample, on an imaging mass calibrator, obtained from the subject, wherein the sample has been labelled with a mass-tagged SBP specific for one or more biomarkers of the disease;
b. performing mass cytometry on the sample to determine the level of the one or more labelling atoms in the mass tag;
c. comparing the level of the one or more labelling atoms with the level determined from an earlier time in the therapy, such as prior to initiation of therapy,
wherein a difference in the level of one or more labelling atoms over time indicates the response of the subject to the therapy. In some embodiments, the method further comprises sampling a fused particle on the imaging mass calibrator to normalise and/or calibrate the level of the one or more labelling atoms. In some instances, response to therapy is indicated by an increase in the level of biomarker over time. In some instances, the disease state is indicated by a decrease in the level of biomarker over time. As the person of skill in the art will appreciate, the specific difference will vary (increase or decrease) and will depend both on the treatment and the disease. In some instances, a conclusion will be drawn on the basis of the relative levels of at least 3, such as at least 5 biomarkers.

### Mass Tagged Detection Reagents

Mass-tagged detection reagents as used herein comprise a number of components. The first is the SBP. The second is the mass tag. The mass tag and the SBP are joined by a linker, formed at least in part of by the conjugation of the mass tag and the SBP. The linkage between the SBP and the mass tag may also comprise a spacer. The mass tag and the SBP can be conjugated together by a range of reaction chemistries. Exemplary conjugation reaction chemistries include thiol maleimide, NHS ester and amine, or click chemistry reactivities (preferably Cu(I)-free chemistries), such as strained alkyne and azide, strained alkyne and nitrone and strained alkene and tetrazine.

### Mass Tags

The mass tag used in the present invention can take a number of forms. Typically, the tag comprises at least one labelling atom. A labelling atom is discussed herein below.

Accordingly, in its simplest form, the mass tag may comprise a metal-chelating moiety which is a metal-chelating group with a metal labelling atom co-ordinated in the ligand. In some instances, detecting only a single metal atom per mass tag may be sufficient. However, in other instances, it may be desirable of each mass tag to contain more than one labelling atom. This can be achieved in a number of ways, as discussed below.

A first means to generate a mass tag that can contain more than one labelling atom is the use of a polymer comprising metal-chelating ligands attached to more than one subunit of the polymer. The number of metal-chelating groups capable of binding at least one metal atom in the polymer can be between approximately 1 and 10,000, such as 5-100, 10-250, 250-5,000, 500-2,500, or 500-1,000. At least one metal atom can be bound to at least one of the metal-chelating groups. The polymer can have a degree of polymerization of between approximately 1 and 10,000, such as 5-100, 10-250, 250-5,000, 500-2,500, or 500-1,000. Accordingly, a polymer based mass tag can comprise between approximately 1 and 10,000, such as 5-100, 10-250, 250-5,000, 500-2,500, or 500-1,000 labelling atoms.

The polymer can be selected from the group consisting of linear polymers, copolymers, branched polymers, graft copolymers, block polymers, star polymers, and hyperbranched polymers. The backbone of the polymer can be derived from substituted polyacrylamide, polymethacrylate, or polymethacrylamide and can be a substituted derivative of a homopolymer or copolymer of acrylamides, methacrylamides, acrylate esters, methacrylate esters, acrylic acid or methacrylic acid. The polymer can be synthesised from the group consisting of reversible addition fragmentation polymerization (RAFT), atom transfer radical polymerization (ATRP), anionic polymerization (including single electron living radical polymerisation), nitroxide-mediated polymerisation (NMP), and photoiniferter-mediated polymerisation (PIMP). The step of providing the polymer can comprise synthesis of the polymer from compounds selected from the group consisting of N-alkyl acrylamides, N,N-dialkyl acrylamides, N-aryl acrylamides, N-alkyl methacrylamides, N,N-dialkyl methacrylamides, N-aryl methacrylamides, methacrylate esters, acrylate esters and functional equivalents thereof.

The polymer can be water soluble. This moiety is not limited by chemical content. However, it simplifies analysis if the skeleton has a relatively reproducible size (for example, length, number of tag atoms, reproducible dendrimer character, etc.). The requirements for stability, solubility, and non-toxicity are also taken into consideration. Thus, the preparation and characterization of a functional water soluble polymer by a synthetic strategy that places many functional groups along the backbone plus a different reactive group (the linking group), that can be used to attach the polymer to a molecule (for example, an SBP), through a linker and optionally a spacer. The size of the polymer is controllable by controlling the polymerisation reaction. Typically the size of the polymer will be chosen so as the radiation of gyration of the polymer is as small as possible, such as between 2 and 11 nanometres. The length of an IgG antibody, an exemplary SBP, is approximately 10 nanometres, and therefore an excessively large polymer tag in relation to the size of the SBP may sterically interfere with SBP binding to its target.

The metal-chelating group that is capable of binding at least one metal atom can comprise at least four acetic acid groups. For instance, the metal-chelating group can be a diethylenetriaminepentaacetate (DTPA) group or a 1,4,7,10-tetraazacyclododecane-1,4,7,10-tetraacetic acid (DOTA) group. Alternative groups include Ethylenediaminetetraacetic acid (EDTA) and ethylene glycol-bis(β-aminoethyl ether)-N,N,N′,N′-tetraacetic acid (EGTA)

The metal-chelating group can be attached to the polymer through an ester or through an amide. Examples of suitable metal-chelating polymers include the X8 and DM3 polymers available from Fluidigm Canada, Inc.

The polymer can be water soluble. Because of their hydrolytic stability, N-alkyl acrylamides, N-alkyl methacrylamides, and methacrylate esters or functional equivalents can be used. A degree of polymerization (DP) of approximately 1 to 1000 (1 to 2000 backbone atoms) encompasses most of the polymers of interest. Larger polymers are in the scope of the invention with the same functionality and are possible as would be understood by practitioners skilled in the art. Typically the degree of polymerization will be between 1 and 10,000, such as 5-100, 10-250, 250-5,000, 500-2,500, or 500-1,000. The polymers may be amenable to synthesis by a route that leads to a relatively narrow polymer dispersity. The polymer may be synthesized by atom transfer radical polymerization (ATRP) or reversible addition-fragmentation (RAFT) polymerization, which should lead to values of Mw (weight average molecular weight)/Mn (number average molecular weight) in the range of 1.1 to 1.2. An alternative strategy involving living anionic polymerization, where polymers with Mw/Mn of approximately 1.02 to 1.05 are obtainable. Both methods permit control over end groups, through a choice of initiating or terminating agents. This allows synthesizing polymers to which the linker can be attached. A strategy of preparing polymers containing functional pendant groups in the repeat unit to which the ligated transition metal unit (for example a Ln unit) can be attached in a later step can be adopted. This embodiment has several advantages. It avoids complications that might arise from carrying out polymerizations of ligand containing monomers.

To minimize charge repulsion between pendant groups, the target ligands for (M³+) should confer a net charge of −1 on the chelate.

Polymers that be used in the invention include:
random copolymer poly(DMA-co-NAS): The synthesis of a 75/25 mole ratio random copolymer of N-acryloxysuccinimide (NAS) with N,N-dimethyl acrylamide (DMA) by RAFT with high conversion, excellent molar mass control in the range of 5000 to 130,000, and with Mw/Mn≈1.1 is reported in Relógio et al. (2004) (Polymer, 45, 8639-49). The active NHS ester is reacted with a metal-chelating group bearing a reactive amino group to yield the metal-chelating copolymer synthesised by RAFT polymerization.
poly(NMAS): NMAS can be polymerised by ATRP, obtaining polymers with a mean molar mass ranging from 12 to 40 KDa with Mw/Mn of approximately 1.1 (see e.g. Godwin et al., 2001; Angew. Chem. Int. Ed, 40: 594-97).
poly(MAA): polymethacrylic acid (PMAA) can be prepared by anionic polymerization of its t-butyl or trimethylsilyl (TMS) ester.
poly(DMAEMA): poly(dimethylaminoethyl methacrylate) (PDMAEMA) can be prepared by ATRP (see Wang et al, 2004, J. Am. Chem. Soc, 126, 7784-85). This is a well-known polymer that is conveniently prepared with mean Mn values ranging from 2 to 35 KDa with Mw/Mn of approximately 1.2 This polymer can also be synthesized by anionic polymerization with a narrower size distribution.
polyacrylamide, or polymethacrylamide.

The metal-chelating groups can be attached to the polymer by methods known to those skilled in the art, for example, the pendant group may be attached through an ester or through an amide. For instance, to a methylacrylate based polymer, the metal-chelating group can be attached to the polymer backbone first by reaction of the polymer with ethylenediamine in methanol, followed by subsequent reaction of DTPA anhydride under alkaline conditions in a carbonate buffer.

A second means is to generate nanoparticles which can act as mass tags. A first pathway to generating such mass tags is the use of nanoscale particles of the metal which have been coated in a polymer. Here, the metal is sequestered and shielded from the environment by the polymer, and does not react when the polymer shell can be made to react e.g. by functional groups incorporated into the polymer shell. The functional groups can be reacted with linker components (optionally incorporating a spacer) to attach click chemistry reagents, so allowing this type of mass tag to plug in to the synthetics strategies discussed above in a simple, modular fashion.

Grafting-to and grafting-from are the two principle mechanism for generating polymer brushes around a nanoparticle. In grafting to, the polymers are synthesised separately, and so synthesis is not constrained by the need to keep the nanoparticle colloidally stable. Here reversible addition-fragmentation chain transfer (RAFT) synthesis has excelled due to a large variety of monomers and easy functionalization. The chain transfer agent (CTA) can be readily used as functional group itself, a functionalized CTA can be used or the polymer chains can be post-functionalized. A chemical reaction or physisorption is used to attach the polymers to the nanoparticle. One drawback of grafting-to is the usually lower grafting density, due to the steric repulsion of the coiled polymer chains during attachment to the particle surface. All grafting-to methods suffer from the drawback that a rigorous workup is necessary to remove the excess of free ligand from the functionalized nanocomposite particle. This is typically achieved by selective precipitation and centrifugation. In the grafting-from approach molecules, like initiators for atomic transfer radical polymerization (ATRP) or CTAs for (RAFT) polymerizations, are immobilized on the particle surface. The drawbacks of this method are the development of new initiator coupling reactions. Moreover, contrary to grafting-to, the particles have to be colloidally stable under the polymerization conditions.

An additional means of generating a mass tag is via the use of doped beads. Chelated lanthanide (or other metal) ions can be employed in miniemulsion polymerization to create polymer particles with the chelated lanthanide ions embedded in the polymer. The chelating groups are chosen, as is known to those skilled in the art, in such a way that the metal chelate will have negligible solubility in water but reasonable solubility in the monomer for miniemulsion polymerization. Typical monomers that one can employ are styrene, methylstyrene, various acrylates and methacrylates, among others as is known to those skilled in the art. For mechanical robustness, the metal-tagged particles have a glass transition temperature (Tg) above room temperature. In some instances, core-shell particles are used, in which the metal-containing particles prepared by miniemulsion polymerization are used as seed particles for a seeded emulsion polymerization to control the nature of the surface functionality. Surface functionality can be introduced through the choice of appropriate monomers for this second-stage polymerization. Additionally, acrylate (and possible methacrylate) polymers are advantageous over polystyrene particles because the ester groups can bind to or stabilize the unsatisfied ligand sites on the lanthanide complexes. An exemplary method for making such doped beads is: (a) combining at least one labelling atom-containing complex in a solvent mixture comprising at least one organic monomer (such as styrene and/or methyl methacrylate in one embodiment) in which the at least one labelling atom-containing complex is soluble and at least one different solvent in which said organic monomer and said at least one labelling atom-containing complex are less soluble, (b) emulsifying the mixture of step (a) for a period of time sufficient to provide a uniform emulsion; (c) initiating polymerization and continuing reaction until a substantial portion of monomer is converted to polymer; and (d) incubating the product of step (c) for a period of time sufficient to obtain a latex suspension of polymeric particles with the at least one labelling atom-containing complex incorporated in or on the particles therein, wherein said at least one labelling atom-containing complex is selected such that upon interrogation of the polymeric mass tag, a distinct mass signal is obtained from said at least one labelling atom. By the use of two or more complexes comprising different labelling atoms, doped beads can be made comprising two or more different labelling atoms. Furthermore, controlling the ratio of the complexes comprising different labelling atoms, allows the production of doped beads with different ratios of the labelling atoms. By use of multiple labelling atoms, and in different radios, the number of distinctively identifiable mass tags is increased. In core-shell beads, this may be achieved by incorporating a first labelling atom-containing complex into the core, and a second labelling atom-containing complex into the shell.

A yet further means is the generation of a polymer that include the labelling atom in the backbone of the polymer rather than as a co-ordinated metal ligand. For instance, Carerra and Seferos (Macromolecules 2015, 48, 297-308) disclose the inclusion of tellurium into the backbone of a polymer. Other polymers incorporating atoms capable as functioning as labelling atoms tin-, antimony- and bismuth-incorporating polymers. Such molecules are discussed inter alia in Priegert et al., 2016 (Chem. Soc. Rev., 45, 922-953).

Thus the mass tag can comprise at least two components: the labelling atoms, and a polymer, which either chelates, contains or is doped with the labelling atom. In addition, the mass tag comprises an attachment group (when not-conjugated to the SBP), which forms part of the chemical linkage between the mass tag and the SBP following reaction of the two components, in a click chemistry reaction in line with the discussion above.

### Labelling Atom

Labelling atoms that can be used with the disclosure include any species that are detectable by MS or OES and that are substantially absent from the unlabelled tissue sample. Thus, for instance, ¹²C atoms would be unsuitable as labelling atoms because they are naturally abundant, whereas ¹¹C could in theory be used for MS because it is an artificial isotope which does not occur naturally. Often the labelling atom is a metal. In preferred embodiments, however, the labelling atoms are transition metals, such as the rare earth metals (the 15 lanthanides, plus scandium and yttrium). These 17 elements (which can be distinguished by OES and MS) provide many different isotopes which can be easily distinguished (by MS). A wide variety of these elements are available in the form of enriched isotopes e.g. samarium has 6 stable isotopes, and neodymium has 7 stable isotopes, all of which are available in enriched form. The 15 lanthanide elements provide at least 37 isotopes that have non-redundantly unique masses. Examples of elements that are suitable for use as labelling atoms include Lanthanum (La), Cerium (Ce), Praseodymium (Pr), Neodymium (Nd), Promethium (Pm), Samarium (Sm), Europium (Eu), Gadolinium, (Gd), Terbium (Tb), Dysprosium (Dy), Holmium (Ho), Erbium (Er), Thulium (Tm), Ytterbium (Yb), Lutetium (Lu), Scandium (Sc), and Yttrium (Y). In addition to rare earth metals, other metal atoms are suitable for detection e.g. gold (Au), platinum (Pt), iridium (Ir), rhodium (Rh), bismuth (Bi), etc. The use of radioactive isotopes is not preferred as they are less convenient to handle and are unstable e.g. Pm is not a preferred labelling atom among the lanthanides.

In order to facilitate time-of-flight (TOF) analysis (as discussed herein) it is helpful to use labelling atoms with an atomic mass within the range 80-250 e.g. within the range 80-210, or within the range 100-200. This range includes all of the lanthanides, but excludes Sc and Y. The range of 100-200 permits a theoretical 101-plex analysis by using different labelling atoms, while taking advantage of the high spectral scan rate of TOF MS. As mentioned above, by choosing labelling atoms whose masses lie in a window above those seen in an unlabelled sample (e.g. within the range of 100-200), TOF detection can be used to provide rapid imaging at biologically significant levels.

Various numbers of labelling atoms can be attached to a single SBP member dependent upon the mass tag used (and so the number of labelling atoms per mass tag) and the number of mass tags that are attached to each SBP). Greater sensitivity can be achieved when more labelling atoms are attached to any SBP member. For example, greater than 10, 20, 30, 40, 50, 60, 70, 80, 90 or 100 labelling atoms can be attached to a SBP member, such as up to 10,000, for instance as 5-100, 10-250, 250-5,000, 500-2,500, or 500-1,000 labelling atoms. As noted above, polymers with a narrow molecular weight distribution containing multiple monomer units may be used, each containing a chelator such as diethylenetriaminepentaacetic acid (DTPA) or DOTA. DTPA, for example, binds 3+ lanthanide ions with a dissociation constant of around 10⁻⁶ M. These polymers can terminate in a thiol which can be used for attaching to a SBP via reaction of that with a maleimide to attach a click chemistry reactivity in line with those discussed above. Other functional groups can also be used for conjugation of these polymers e.g. amine-reactive groups such as N-hydroxy succinimide esters, or groups reactive against carboxyls or against an antibody's glycosylation. Any number of polymers may bind to each SBP. Specific examples of polymers that may be used include straight-chain (“X8”) polymers or third-generation dendritic (“DN3”) polymers, both available as MaxPar™ reagents. Use of metal nanoparticles can also be used to increase the number of atoms in a label, as also discussed above.

In some embodiments, all labelling atoms in a mass tag are of the same atomic mass. Alternatively, a mass tag can comprise labelling atoms of differing atomic mass. Accordingly, in some instances, a labelled sample may be labelled with a series of mass-tagged SBPs each of which comprises just a single type of labelling atom (wherein each SBP binds its cognate target and so each kind of mass tag is localised on the sample to a specific e.g. antigen). Alternatively, in some instance, a labelled sample may be labelled with a series of mass-tagged SBPs each of which comprises a mixture of labelling atoms. In some instances, the mass-tagged SBPs used to label the sample may comprise a mix of those with single labelling atom mass tags and mixes of labelling atoms in their mass tags.

### Spacer

As noted above, in some instances, the SBP is conjugated to a mass tag through a linker which comprises a spacer. There may be a spacer between the SBP and the click chemistry reagent (e.g. between the SBP and the strained cycloalkyne (or azide); strained cycloalkene (or tetrazine); etc.). There may be a spacer between the between the mass tag and the click chemistry reagent (e.g. between the mass tag and the azide (or strained cycloalkyne); tetrazine (or strained cycloalkene); etc.). In some instances there may be a spacer both between the SNP and the click chemistry reagent, and the click chemistry reagent and the mass tag.

The spacer might be a polyethylene glycol (PEG) spacer, a poly(N-vinylpyrolide) (PVP) spacer, a polyglycerol (PG) spacer, poly(N-(2-hydroxylpropyl)methacrylamide) spacer, or a polyoxazoline (POZ, such as polymethyloxazoline, polyethyloxazoline or polypropyloxazoline) or a C5-C20 non-cyclic alkyl spacer. For example, the spacer may be a PEG spacer with 3 or more, 4 or more, 5 or more, 6 or more, 7 or more, 8 or more, 9 or more, 10 or more, 11 or more, 12 or more, 15 or more of 20 or more EG (ethylene glycol) units. The PEG linker may have from 3 to 12 EG units, from 4 to 10, or may have 4, 5, 6, 7, 8, 9, or 10 EG units. The linker may include cystamine or derivatives thereof, may include one or more disulfide groups, or may be any other suitable linker known to one of skill in the art.

Spacers may be beneficial to minimize the steric effect of the mass tag on the SBP to which is conjugated. Hydrophilic spacers, such as PEG based spacers, may also act to improve the solubility of the mass-tagged SBP and act to prevent aggregation.

### SBPs (Specific Binding Pair Members)

Mass cytometry, including imaging mass cytometry is based on the principle of specific binding between members of specific binding pairs. The mass tag is linked to a specific binding pair member, and this localises the mass tag to the target/analyte which is the other member of the pair. Specific binding does not require binding to just one molecular species to the exclusion of others, however. Rather it defines that the binding is not-nonspecific, i.e. not a random interaction. An example of an SBP that binds to multiple targets would therefore be an antibody which recognises an epitope that is common between a number of different proteins. Here, binding would be specific, and mediated by the complementary determining regions (CDRs) of the antibody, but multiple different proteins would be detected by the antibody. The common epitopes may be naturally occurring, or the common epitope could be an artificial tag, such as a FLAG tag. Similarly, for nucleic acids, a nucleic acid of defined sequence may not bind exclusively to a fully complementary sequence, but varying tolerances of mismatch can be introduced under the use of hybridisation conditions of a differing stringencies, as would be appreciated by one of skill in the art. Nonetheless, this hybridisation is not non-specific, because it is mediated by homology between the SBP nucleic acid and the target analyte. Similarly, ligands can bind specifically to multiple receptors, a facile example being TNFα which binds to both TNFR1 and TNFR2.

The SBP may comprise any of the following: a nucleic acid duplex; an antibody/antigen complex; a receptor/ligand pair; or an aptamer/target pair. Thus a labelling atom can be attached to a nucleic acid probe which is then contacted with a tissue sample so that the probe can hybridise to complementary nucleic acid(s) therein e.g. to form a DNA/DNA duplex, a DNA/RNA duplex, or a RNA/RNA duplex. Similarly, a labelling atom can be attached to an antibody which is then contacted with a tissue sample so that it can bind to its antigen. A labelling atom can be attached to a ligand which is then contacted with a tissue sample so that it can bind to its receptor. A labelling atom can be attached to an aptamer ligand which is then contacted with a tissue sample so that it can bind to its target. Thus, labelled SBP members can be used to detect a variety of targets in a sample, including DNA sequences, RNA sequences, proteins, sugars, lipids, or metabolites.

The mass-tagged SBP therefore can be a protein or peptide, or a polynucleotide or oligonucleotide.

Examples of protein SBPs include an antibody or antigen binding fragment thereof, a monoclonal antibody, a polyclonal antibody, a bispecific antibody, a multispecific antibody, an antibody fusion protein, scFv, antibody mimetic, avidin, streptavidin, neutravidin, biotin, or a combination thereof, wherein optionally the antibody mimetic comprises a nanobody, affibody, affilin, affimer, affitin, alphabody, anticalin, avimer, DARPin, Fynomer, kunitz domain peptide, monobody, or any combination thereof, a receptor, such as a receptor-Fc fusion, a ligand, such as a ligand-Fc fusion, a lectin, for example an agglutinin such as wheat germ agglutinin.

The peptide may be a linear peptide, or a cyclical peptide, such as a bicyclic peptide. One example of a peptide that can be used is Phalloidin.

A polynucleotide or oligonucleotide generally refers to a single- or double-stranded polymer of nucleotides containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), yPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding.

### Antibody SBP Members

In a typical embodiment, the labelled SBP member is an antibody. Labelling of the antibody can be achieved through conjugation of one or more labelling atom binding molecules to the antibody, by attachment of a mass tag using e.g. NHS-amine chemistry, sulfhydryl-maleimide chemistry, or the click chemistry (such as strained alkyne and azide, strained alkyne and nitrone, strained alkene and tetrazine etc.). Antibodies which recognise cellular proteins that are useful for imaging are already widely available for IHC usage, and by using labelling atoms instead of current labelling techniques (e.g. fluorescence) these known antibodies can be readily adapted for use in methods disclosure herein, but with the benefit of increasing multiplexing capability. Antibodies can recognise targets on the cell surface or targets within a cell. Antibodies can recognise a variety of targets e.g. they can specifically recognise individual proteins, or can recognise multiple related proteins which share common epitopes, or can recognise specific post-translational modifications on proteins (e.g. to distinguish between tyrosine and phosphor-tyrosine on a protein of interest, to distinguish between lysine and acetyl-lysine, to detect ubiquitination, etc.). After binding to its target, labelling atom(s) conjugated to an antibody can be detected to reveal the location of that target in a sample.

The labelled SBP member will usually interact directly with a target SBP member in the sample. In some embodiments, however, it is possible for the labelled SBP member to interact with a target SBP member indirectly e.g. a primary antibody may bind to the target SBP member, and a labelled secondary antibody can then bind to the primary antibody, in the manner of a sandwich assay. Usually, however, the method relies on direct interactions, as this can be achieved more easily and permits higher multiplexing. In both cases, however, a sample is contacted with a SBP member which can bind to a target SBP member in the sample, and at a later stage label attached to the target SBP member is detected.

### Nucleic Acid SBPs, and Labelling Methodology Modifications

RNA is another biological molecule which the methods and apparatus disclosed herein are capable of detecting in a specific, sensitive and if desired quantitative manner. In the same manner as described above for the analysis of proteins, RNAs can be detected by the use of a SBP member labelled with an elemental tag that specifically binds to the RNA (e.g. an poly nucleotide or oligonucleotide of complementary sequence as discussed above, including a locked nucleic acid (LNA) molecule of complementary sequence, a peptide nucleic acid (PNA) molecule of complementary sequence, a plasmid DNA of complementary sequence, an amplified DNA of complementary sequence, a fragment of RNA of complementary sequence and a fragment of genomic DNA of complementary sequence). RNAs include not only the mature mRNA, but also the RNA processing intermediates and nascent pre-mRNA transcripts.

In certain embodiments, both RNA and protein are detected using methods of the claimed invention.

To detect RNA, cells in biological samples as discussed herein may be prepared for analysis of RNA and protein content using the methods and apparatus described herein. In certain aspects, cells are fixed and permeabilized prior to the hybridization step. Cells may be provided as fixed and/or permeabilized. Cells may be fixed by a crosslinking fixative, such as formaldehyde, glutaraldehyde. Alternatively or in addition, cells may be fixed using a precipitating fixative, such as ethanol, methanol or acetone. Cells may be permeabilized by a detergent, such as surfactants comprising polyethylene glycol (e.g., Triton X-100, Tween-20), Saponin (a group of amphipathic glycosides), or chemicals such as methanol or acetone. In certain cases, fixation and permeabilization may be performed with the same reagent or set of reagents. Fixation and permeabilization techniques are discussed by Jamur et al. in “Permeabilization of Cell Membranes” (Methods Mol. Biol., 2010).

Detection of target nucleic acids in the cell, or “in-situ hybridization” (ISH), has previously been performed using fluorophore-tagged oligonucleotide probes. As discussed herein, mass-tagged oligonucleotides, coupled with ionization and mass spectrometry, can be used to detect target nucleic acids in the cell. Methods of in-situ hybridization are known in the art (see Zenobi et al. “Single-Cell Metabolomics: Analytical and Biological Perspectives,” Science vol. 342, no. 6163, 2013). Hybridization protocols are also described in U.S. Pat. No. 5,225,326 and US Pub. No. 2010/0092972 and 2013/0164750, which are incorporated herein by reference.

Prior to hybridization, cells present in suspension or immobilized on a solid support may be fixed and permeabilized as discussed earlier. Permeabilization may allow a cell to retain target nucleic acids while permitting target hybridization nucleotides, amplification oligonucleotides, and/or mass-tagged oligonucleotides to enter the cell. The cell may be washed after any hybridization step, for example, after hybridization of target hybridization oligonucleotides to nucleic acid targets, after hybridization of amplification oligonucleotides, and/or after hybridization of mass-tagged oligonucleotides.

Cells can be in suspension for all or most of the steps of the method, for ease of handling. However, the methods are also applicable to cells in solid tissue samples (e.g., tissue sections) and/or cells immobilized on a solid support (e.g., a slide or other surface). Thus, sometimes, cells can be in suspension in the sample and during the hybridization steps. Other times, the cells are immobilized on a solid support during hybridization.

Target nucleic acids include any nucleic acid of interest and of sufficient abundance in the cell to be detected by the subject methods. Target nucleic acids may be RNAs, of which a plurality of copies exist within the cell. For example, 10 or more, 20 or more, 50 or more, 100 or more, 200 or more, 500 or more, or 1000 or more copies of the target RNA may be present in the cell. A target RNA may be a messenger NA (mRNA), ribosomal RNA (rRNA), transfer RNA (tRNA), small nuclear RNA (snRNA), small interfering RNA (siRNA), long noncoding RNA (IncRNA), or any other type of RNA known in the art. The target RNA may be 20 nucleotides or longer, 30 nucleotides or longer, 40 nucleotides or longer, 50 nucleotides or longer, 100 nucleotides or longer, 200 nucleotides or longer, 500 nucleotides or longer, 1000 nucleotides or longer, between 20 and 1000 nucleotides, between 20 and 500 nucleotides in length, between 40 and 200 nucleotides in length, and so forth.

In certain embodiments, a mass-tagged oligonucleotide may be hybridized directly to the target nucleic acid sequence. However, hybridization of additional oligonucleotides may allow for improved specificity and/or signal amplification.

In certain embodiments, two or more target hybridization oligonucleotides may be hybridized to proximal regions on the target nucleic acid, and may together provide a site for hybridization of an additional oligonucleotides in the hybridization scheme.

In certain embodiments, the mass-tagged oligonucleotide may be hybridized directly to the two or more target hybridization oligonucleotides. In other embodiments, one or more amplification oligonucleotides may be added, simultaneously or in succession, so as to hybridize the two or more target hybridization oligonucleotides and provide multiple hybridization sites to which the mass-tagged oligonucleotide can bind. The one or more amplification oligonucleotides, with or without the mass-tagged oligonucleotide, may be provided as a multimer capable of hybridizing to the two or more target hybridization oligonucleotides.

While the use of two or more target hybridization oligonucleotides improves specificity, the use of amplification oligonucleotides increases signal. Two target hybridization oligonucleotides are hybridized to a target RNA in the cell. Together, the two target hybridization oligonucleotides provide a hybridization site to which an amplification oligonucleotide can bind. Hybridization and/or subsequent washing of the amplification oligonucleotide may be performed at a temperature that allows hybridization to two proximal target hybridization oligonucleotides, but is above the melting temperature of the hybridization of the amplification oligonucleotide to just one target hybridization oligonucleotide. The first amplification oligonucleotide provides multiple hybridization sites, to which second amplification oligonucleotides can be bound, forming a branched pattern. Mass-tagged oligonucleotides may bind to multiple hybridization sites provided by the second amplification nucleotides. Together, these amplification oligonucleotides (with or without mass-tagged oligonucleotides) are referred to herein as a “multimer”. Thus the term “amplification oligonucleotide” includes oligonucleotides that provides multiple copies of the same binding site to which further oligonucleotides can anneal. By increasing the number of binding sites for other oligonucleotides, the final number of labels that can be found to a target is increased. Thus, multiple labelled oligonucleotides are hybridized, indirectly, to a single target RNA. This is enables the detection of low copy number RNAs, by increasing the number of detectable atoms of the element used per RNA.

One particular method for performing this amplification comprises using the RNAscope® method from Advanced cell diagnostics, as discussed in more detail below. A further alternative is the use of a method that adapts the QuantiGene® FlowRNA method (Affymetrix eBioscience). The assay is based on oligonucleotide pair probe design with branched DNA (bDNA) signal amplification. There are more than 4,000 probes in the catalog or custom sets can be requested at no additional charge. In line with the previous paragraph, the method works by hybridization of target hybridization oligonucleotides to the target, followed by the formation of a branched structure comprising first amplification oligonucleotides (termed preamplification oligonucleotides in the QuantiGene® method) to form a stem to which multiple second amplification oligonucleotides can anneal (termed simply amplification oligonucleotides in the QuantiGene® method). Multiple mass-tagged oligonucleotides can then bind.

Another means of amplification of the RNA signal relies on the rolling circle means of amplification (RCA). There are various means by which this amplification system can be introduced into the amplification process. In a first instance, a first nucleic acid is used as the hybridisation nucleic acid wherein the first nucleic acid is circular. The first nucleic acid can be single stranded or may be double-stranded. It comprises as sequence complementary to the target RNA. Following hybridisation of the first nucleic acid to the target RNA, a primer complementary to the first nucleic acid is hybridised to the first nucleic acid, and used for primer extension using a polymerase and nucleic acids, typically exogenously added to the sample. In some instances, however, when the first nucleic acid is added to sample, it may already have the primer for extension hybridised to it. As a result of the first nucleic acid being circular, once the primer extension has completed a full round of replication, the polymerase can displace the primer and extension continues (i.e. without 5′43′ exonuclase activity), producing linked further and further chained copies of the complement of the first nucleic acid, thereby amplifying that nucleic acid sequence. Oligonucleotides comprising an elemental tag (RNA or DNA, or LNA or PNA and the like) as discussed above) may therefore be hybridised to the chained copies of the complement of the first nucleic acid. The degree of amplification of the RNA signal can therefore be controlled by the length of time allotted for the step of amplification of the circular nucleic acid.

In another application of RCA, rather than the first, e.g., oligonucleotide that hybridises to the target RNA being circular, it may be linear, and comprise a first portion with a sequence complementary to its target and a second portion which is user-chosen. A circular RCA template with sequence homologous to this second portion may then be hybridised to this the first oligonucleotide, and RCA amplification carried out as above. The use of a first, e.g., oligonucleotide having a target specific portion and user-chosen portion is that the user-chosen portion can be selected so as to be common between a variety of different probes. This is reagent-efficient because the same subsequent amplification reagents can be used in a series of reactions detecting different targets. However, as understood by the skilled person, when employing this strategy, for individual detection of specific RNAs in a multiplexed reaction, each first nucleic acid hybridising to the target RNA will need to have a unique second sequence and in turn each circular nucleic acid should contain unique sequence that can be hybridised by the labelled oligonucleotide. In this manner, signal from each target RNA can be specifically amplified and detected.

Other configurations to bring about RCA analysis will be known to the skilled person. In some instances, to prevent the first, e.g., oligonucleotide dissociating from the target during the following amplification and hybridisation steps, the first, e.g., oligonucleotide may be fixed following hybridisation (such as by formaldehyde).

Further, hybridisation chain reaction (HCR) may be used to amplify the RNA signal (see, e.g., Choi et al., 2010, Nat. Biotech, 28:1208-1210). Choi explains that an HCR amplifier consists of two nucleic acid hairpin species that do not polymerise in the absence of an initiator. Each HCR hairpin consists of an input domain with an exposed single-stranded toehold and an output domain with a single-stranded toehold hidden in the folded hairpin. Hybridization of the initiator to the input domain of one of the two hairpins opens the hairpin to expose its output domain. Hybridization of this (previously hidden) output domain to the input domain of the second hairpin opens that hairpin to expose an output domain identical in sequence to the initiator. Regeneration of the initiator sequence provides the basis for a chain reaction of alternating first and second hairpin polymerization steps leading to formation of a nicked double-stranded ‘polymer’. Either or both of the first and second hairpins can be labelled with an elemental tag in the application of the methods and apparatus disclosed herein. As the amplification procedure relies on output domains of specific sequence, various discrete amplification reactions using separate sets of hairpins can be performed independently in the same process. Thus this amplification also permits amplification in multiplex analyses of numerous RNA species. As Choi notes, HCR is an isothermal triggered self-assembly process. Hence, hairpins should penetrate the sample before undergoing triggered self-assembly in situ, suggesting the potential for deep sample penetration and high signal-to-background ratios

Hybridization may include contacting cells with one or more oligonucleotides, such as target hybridization oligonucleotides, amplification oligonucleotides, and/or mass-tagged oligonucleotides, and providing conditions under which hybridization can occur. Hybridization may be performed in a buffered solution, such as saline sodium-citrate (SCC) buffer, phosphate-buffered saline (PBS), saline-sodium phosphate-EDTA (SSPE) buffer, TNT buffer (having Tris-HCl, sodium chloride and Tween 20), or any other suitable buffer. Hybridization may be performed at a temperature around or below the melting temperature of the hybridization of the one or more oligonucleotides.

Specificity may be improved by performing one or more washes following hybridization, so as to remove unbound oligonucleotide. Increased stringency of the wash may improve specificity, but decrease overall signal. The stringency of a wash may be increased by increasing or decreasing the concentration of the wash buffer, increasing temperature, and/or increasing the duration of the wash. RNAse inhibitor may be used in any or all hybridization incubations and subsequent washes.

A first set of hybridization probes, including one or more target hybridizing oligonucleotides, amplification oligonucleotides and/or mass-tagged oligonucleotides, may be used to label a first target nucleic acid. Additional sets of hybridization probes may be used to label additional target nucleic acids. Each set of hybridization probes may be specific for a different target nucleic acid. The additional sets of hybridization probes may be designed, hybridized and washed so as to reduce or prevent hybridization between oligonucleotides of different sets. In addition, the mass-tagged oligonucleotide of each set may provide a unique signal. As such, multiple sets of oligonucleotides may be used to detect 2, 3, 5, 10, 15, 20 or more distinct nucleic acid targets.

Sometimes, the different nucleic acids detected are splice variants of a single gene. The mass-tagged oligonucleotide can be designed to hybridize (directly or indirectly through other oligonucleotides as explained below) within the sequence of the exon, to detect all transcripts containing that exon, or may be designed to bridge the splice junctions to detect specific variants (for example, if a gene had three exons, and two splice variants—exons 1-2-3 and exons 1-3—then the two could be distinguished: variant 1-2-3 could be detected specifically by hybridizing to exon 2, and variant 1-3 could be detected specifically by hybridizing across the exon 1-3 junction.

### Histochemical Stains

The histochemical stain reagents having one or more intrinsic metal atoms may be combined with other reagents and methods of use as described herein. For example, histochemical stains may be colocalized (e.g., at cellular or subcellular resolution) with metal containing drugs, metal-labelled antibodies, and/or accumulated heavy metals. In certain aspects, one or more histochemical stains may be used at lower concentrations (e.g., less than half, a quarter, a tenth, etc.) from what is used for other methods of imaging (e.g., fluorescence microscopy, light microscopy, or electron microscopy).

To visualize and identify structures, a broad spectrum of histological stains and indicators are available and well characterized. The metal-containing stains have a potential to influence the acceptance of the imaging mass cytometry by pathologists. Certain metal containing stains are well known to reveal cellular components, and are suitable for use in the subject invention. Additionally, well defined stains can be used in digital image analysis providing contrast for feature recognition algorithms. These features are strategically important for the development of imaging mass cytometry.

Often, morphological structure of a tissue section can be contrasted using affinity products such as antibodies. They are expensive and require additional labelling procedure using metal-containing tags, as compared to using histochemical stains. This approach was used in pioneering works on imaging mass cytometry using antibodies labelled with available lanthanide isotopes thus depleting mass (e.g. metal) tags for functional antibodies to answer a biological question.

The subject invention expands the catalog of available isotopes including such elements as Ag, Au, Ru, W, Mo, Hf, Zr (including compounds such as Ruthenium Red used to identify mucinous stroma, Trichrome stain for identification of collagen fibers, osmium tetroxide as cell counterstain). Silver staining is used in karyotyping. Silver nitrate stains the nucleolar organization region (NOR)-associated protein, producing a dark region wherein the silver is deposited and denoting the activity of rRNA genes within the NOR. Adaptation to IMC may require that the protocols (e.g., oxidation with potassium permanganate and a silver concentration of 1% during) be modified for use lower concentrations of silver solution, e.g., less than 0.5%, 0.01%, or 0.05% silver solution.

Autometallographic amplification techniques have evolved into an important tool in histochemistry. A number of endogenous and toxic heavy metals form sulfide or selenide nanocrystals that can be autocatalytically amplified by reaction with Ag ions. The larger Ag nanocluster can then be readily visualized by IMC. At present, robust protocols for the silver amplified detection of Zn—S/Se nanocrystals have been established as well as detection of selenium through formation of silver-selenium nanocrystals. In addition, commercially available quantum dots (detection of Cd) are also autocatalytically active and may be used as histochemical labels.

Aspects of the subject invention may include histochemical stains and their use in imaging by elemental mass spectrometry. Any histochemical stain resolvable by elemental mass spectrometry may be used in the subject invention. In certain aspects, the histochemical stain includes one or more atoms of mass greater than a lower mass cut-off of the detector of an mass spectrometer used to image the sample, such as greater than 60 amu, 80 amu, 100 amu, or 120 amu. For example, the histochemical stain may include a metal tag (e.g., metal atom) as described herein. The metal atom may be chelated to the histochemical stain, or covalently bound within the chemical structure of the histochemical stain. In certain aspects, the histochemical stain may be an organic molecule. Histochemical stains may be polar, hydrophobic (e.g., lipophilic), ionic or may comprise groups with different properties. In certain aspects, a histochemical stain may comprise more than one chemical.

Histochemical stains include small molecules of less than 2000, 1500, 1000, 800, 600, 400, or 200 amu. Histochemical stains may bind to the sample through covalent or non-covalent (e.g., ionic or hydrophobic) interactions. Histochemical stains may provide contrast to resolve the morphology of the biological sample, for example, to help identify individual cells, intracellular structures, and/or extracellular structures. Intracellular structures that may be resolved by histochemical stains include cell membrane, cytoplasm, nucleus, Golgi body, ER, mitochondria, and other cellular organelles. Histochemical stains may have an affinity for a type of biological molecule, such as nucleic acids, proteins, lipids, phospholipids or carbohydrates. In certain aspects, a histochemical stain may bind a molecule other than DNA. Suitable histochemical stains also include stains that bind extracellular structures (e.g., structures of the extracellular matrix), including stroma (e.g., mucosal stroma), basement membrane, interstitial stroma, proteins such as collage or elastin, proteoglycans, non-proteoglycan polysaccharides, extracellular vesicles, fibronectin, laminin, and so forth.

In certain aspects, histochemical stains and/or metabolic probes may indicate a state of a cell or tissue. For example, histochemical stains may include vital stains such as cisplatin, eosin, and propidium iodide. Other histochemical stains may stain for hypoxia, e.g., may only bind or deposit under hypoxic conditions. Probes such as Iododeoxyuridine (IdU) or a derivative thereof, may stain for cell proliferation. In certain aspects, the histochemical stain may not indicate the state of the cell or tissue. Probes that detect cell state (e.g., viability, hypoxia and/or cell proliferation) but are administered in-vivo (e.g., to a living animal or cell culture) be used in any of the subject methods but do not qualify as histochemical stains.

Histochemical stains may have an affinity for a type of biological molecule, such as nucleic acids (e.g., DNA and/or RNA), proteins, lipids, phospholipids, carbohydrates (e.g., sugars such as mono-saccharides or di-saccharides or polyols; oligosaccharides; and/or polysaccharides such as starch or glycogen), glycoproteins, and/or glycolipids. In certain aspects the histochemical stain may be a counterstain.

The following are examples of specific histochemical stains and their use in the subject methods:

Ruthenium Red stain as a metal-containing stain for mucinous stroma detection may be used as follows: Immunostained tissue (e.g., de-paraffinized FFPE or cryosection) may be treated with 0.0001-0.5%, 0.001-0.05%, less than 0.1%, less than 0.05%, or around 0.0025% Ruthenium Red (e.g., for at least 5 minutes, at least 10 minutes, at least 30 minutes, or around 30 min at 4-42° C., or around room temperature). The biological sample may be rinsed, for example with water or a buffered solution. Tissue may then be dried before imaging by elemental mass spectrometry.

Phosphotungstic Acid (e.g., as a Trichrome stain) may be used as a metal-containing stain for collagen fibers. Tissue sections on slides (de-paraffinized FFPE or cryosection) may be fixed in Bouin's fluid (e.g., for at least 5 minutes, at least 10 minutes, at least 30 minutes, or around 30 minutes at 4-42° C. or around room temperature). The sections may then be treated with 0.0001%-0.01%, 0.0005%-0.005%, or around 0.001% Phosphotangstic Acid for (e.g., for at least 5 minutes, at least 10 minutes, at least 30 minutes, or around 15 minutes at 4-42° C. or around room temperature). Sample may then be rinsed with water and/or buffered solution, and optionally dried, prior to imaging by elemental mass spectrometry. Trichrome stain may be used at a dilution (e.g., 5 fold, 10 fold, 20 fold, 50 fold or great dilution) compared to concentrations used for imaging by light (e.g., fluorescence) microscopy.

In some embodiments, the histochemical stain is an organic molecule. In some embodiments, the second metal is covalently bound. In some embodiments, the second metal is chelated. In some embodiments, the histochemical stain specifically binds cell membrane. In some embodiments, the histochemical stain is osmium tetroxide. In some embodiments, the histochemical stain is lipophilic. In some embodiments, the histochemical stain specifically binds an extracellular structure. In some embodiments, the histochemical stain specifically binds extracellular collagen. In some embodiments, the histochemical stain is a trichrome stain comprising phosphotungstic/phosphomolybdic acid. In some embodiments, trichrome stain is used after contacting the sample with the antibody, such as at a lower concentration than would be used for optical imaging, for instance wherein the concentration is a 50 fold dilution of trichrome stain or greater.

### Metal-Containing Drugs

Metals in medicine is a new and exciting field in pharmacology. Little is known about the cellular structures that are involved in transiently storing metal ions prior to their incorporation into metalloproteins, nucleic acid metal complexes or metal-containing drugs or the fate of metal ions upon protein or drug degradation. An important first step towards unravelling the regulatory mechanisms involved in trace metal transport, storage, and distribution represents the identification and quantitation of the metals, ideally in context of their native physiological environment in tissues, cells, or even at the level of individual organelles and subcellular compartments. Histological studies are typically carried out on thin sections of tissue or with cultured cells.

A number of metal-containing drugs are being used for treatment of various diseases, however not enough is known about their mechanism of action or biodistribution: cisplatin, ruthenium imidazole, metallocene-based anti-cancer agents with Mo, tungstenocenes with W, B-diketonate complexes with Hf or Zr, auranofin with Au, polyoxomolybdate drugs. Many metal complexes are used as MRI contrast agents (Gd(III) chelates). Characterization of the uptake and biodistribution of metal-based anti-cancer drugs is of critical importance for understanding and minimizing the underlying toxicity.

The atomic masses of certain metals present in drugs fall into the range of mass cytometry. Specifically, cisplatin and others with Pt complexes (iproplatin, lobplatin) are extensively used as a chemotherapeutic drug for treating a wide range of cancers. The nephrotoxicity and myelotoxicity of platinum-based anti-cancer drugs is well known. With the methods and reagents described herein, their subcellular localization within tissue sections, and colocalization with mass- (e.g. metal-) tagged antibodies and/or histochemical stains can now be examined. Chemotherepeutic drugs may be toxic to certain cells, such as proliferating cells, through direct DNA damage, inhibition of DNA damage repair pathways, radioactivity, and so forth. In certain aspects, chemotherapeutic drugs may be targeted to tumor through an antibody intermediate.

In certain aspects, the metal containing drug is a chemotherapeutic drug. Subject methods may include administering the metal containing drug to a living animal, such as an animal research model or human patient as previously described, prior to obtaining the biological sample. The biological sample may be, for example, a biopsy of cancerous tissue or primary cells. Alternatively, the metal containing drug may be added directly to the biological sample, which may be an immortalized cell line or primary cells. When the animal is a human patient, the subject methods may include adjusting a treatment regimen that includes the metal containing drug, based on detecting the distribution of the metal containing drug.

The method step of detecting the metal containing drug may include subcellular imaging of the metal containing drug by elemental mass spectrometry, and may include detecting the retention of the metal containing drug in an intracellular structure (such as membrane, cytoplasm, nucleus, Golgi body, ER, mitochondria, and other cellular organelles) and/or extracellular structure (such as including stroma, mucosal stroma, basement membrane, interstitial stroma, proteins such as collage or elastin, proteoglycans, non-proteoglycan polysaccharides, extracellular vesicles, fibronectin, laminin, and so forth).

A histochemical stain and/or mass- (e.g. metal-) tagged SBP that resolves (e.g., binds to) one or more of the above structures may be colocalized with the metal containing drug to detected retention of the drug at specific intracellular or extracellular structures. For example, a chemotherapeutic drug such as cisplatin may be colocalized with a structure such as collagen. Alternatively or in addition, the localization of the drug may be related to presence of a marker of cell viability, cell proliferation, hypoxia, DNA damage response, or immune response.

In some embodiments, the metal containing drug comprises a non-endogenous metal, such as wherein the non-endogenous metal is platinum, palladium, cerium, cadmium, silver or gold. In certain aspects, the metal containing drug is one of cisplatin, ruthenium imidazole, metallocene-based anti-cancer agents with Mo, tungstenocenes with W, B-diketonate complexes with Hf or Zr, auranofin with Au, polyoxomolybdate drugs, N-myristoyltransferase-1 inhibitor (Tris(dibenzylideneacetone) dipalladium) with Pd, or a derivative thereof. For example the drug may comprise Pt, and may be, for example, cisplatin, carboplatin, oxaliplatin, iproplatin, lobaplatin or a derivative thereof. The metal containing drug may include a non-endogenous metal, such as platinum (Pt), ruthenium (Ru), molybdenum (Mo), tungsten (W), hafnium (Hf), zirconium (Zr), gold (Au), gadolinium (Gd), palladium (Pd) or an isotope thereof. Gold compounds (Auranofin, for example) and gold nanoparticle bioconjugates for photothermal therapy against cancer can be identified in tissue sections.

### Accumulated Heavy Metals

Exposure to heavy metals can occur though injection of food or water, contact through skin, or aerosol intake. Heavy metals may accumulate in soft tissues of the body, such that prolonged exposure has serious health effects. In certain aspect, the heavy metal may be accumulated in vivo, either through controlled exposure in an animal research model or though environmental exposure in a human patient. The heavy metal may be a toxic heavy metal, such as Arsenic (As), Lead (Pb), Antimony (Sb), Bismuth (Bi), Cadmium (Cd), Osmium (Os), Thallium (TI), or Mercury (Hg).

The subject methods may be used to diagnose and/or characterize heavy metal poisoning in a human patient, determine a treatment regimen for a human patient, or characterize accumulation and/or treatment of heavy metals in an animal research model.

### Samples

Certain aspects of the disclosure provide a method of analysing a biological sample, such as imaging a biological sample. Such samples can comprise a plurality of cells, a plurality of these cells can be subjected to mass imaging, such as imaging mass cytometry (IMC) in order to provide an image of these cells in the sample. In general, the invention can be used to analyse tissue samples which are now studied by FACS or immunohistochemistry (IHC) techniques, but with the use of labelling atoms which are suitable for detection by mass spectrometry (MS) or optical emission spectrometry (OES).

Any suitable tissue sample can be used in the methods described herein. For example, the tissue can include tissue from one or more of epithelium, muscle, nerve, skin, intestine, pancreas, kidney, brain, liver, blood, bone marrow, buccal swipes, cervical swipes, or any other tissue. Other bodily fluids can be a sample too, such as ascites, lung fluid, spinal fluid, amniotic fluid, blood plasma, blood serum, extracellular fluid, exudate, faeces, urine. Cell lysates can also be analysed as can cell culture supernatants, bacterial culture and/or lysate, viral culture and or culture supernatant. The biological sample may be an immortalized cell line or primary cells obtained from a living subject. For diagnostic, prognostic or experimental (e.g., drug development) purposes the tissue can be from a tumor. In some embodiments, a sample may be from a known tissue, but it might be unknown whether the sample contains tumor cells. Imaging can reveal the presence of targets which indicate the presence of a tumor, thus facilitating diagnosis. Tissue from a tumor may comprise immune cells that are also characterized by the subject methods, and may provide insight into the tumor biology. The tissue sample may comprise formalin-fixed, paraffin-embedded (FFPE) tissue. The tissues can be obtained from any living multicellular organism, such as a mammal, an animal research model (e.g., of a particular disease, such as an immunodeficient rodent with a human tumor xenograft), or a human patient.

The sample may also consist or comprise analytes which have been immobilised to the sample carrier from solution (e.g. in array form). Accordingly, the sample may comprise a cell, a population of cells, a protein solution, a peptide solution, a nucleic acid solution, and carbohydrate solution, a solution comprising multiple macromolecule types, for example a solution of proteins and nucleic acids and a solution of proteins, nucleic acids and carbohydrates, and a solution comprising multiple macromolecule types and cells, for example a solution of proteins, nucleic acids and cells, and a solution of proteins, nucleic acids, carbohydrates and cells. Thus, the sample may be a tissue homogenate, tissue fluid, bodily fluid, ascites, lung fluid, spinal fluid, amniotic fluid, bone marrow aspirate, blood plasma, blood serum, exudate, faeces, urine, cell lysate, cell culture supernatant, extracellular fluid, bacterial lysate, viral supernatant, any combination thereof or other biological fluids.

The tissue sample may be a section e.g. having a thickness within the range of 2-10 μm, such as between 4-6 μm. Techniques for preparing such sections are well known from the field of IHC e.g. using microtomes, including dehydration steps, fixation, embedding, permeabilization, sectioning etc. Thus, a tissue may be chemically fixed and then sections can be prepared in the desired plane. Cryosectioning or laser capture microdissection can also be used for preparing tissue samples. Samples may be permeabilised e.g. to permit of reagents for labelling of intracellular targets (see above).

The size of a tissue sample to be analysed will be similar to current IHC methods, although the maximum size will be dictated by the laser ablation apparatus, and in particular by the size of sample which can fit into its sample chamber. A size of up to 5 mm×5 mm is typical, but smaller samples (e.g. 1 mm×1 mm) are also useful (these dimensions refer to the size of the section, not its thickness).

In addition to being useful for imaging tissue samples, the disclosure can instead be used for imaging of cellular samples such as monolayers of adherent cells or of cells which are immobilised on a solid surface (as in conventional immunocytochemistry). These embodiments are particularly useful for the analysis of adherent cells that cannot be easily solubilized for cell-suspension mass cytometry. Thus, as well as being useful for enhancing current immunohistochemical analysis, the disclosure can be used to enhance immunocytochemistry.

Antibodies and/or histochemical stains, as described above, may allow monitoring of tissue state, such as cell proliferation (e.g., using the target Ki-67 or marker IdU), DNA damage response (e.g., using a marker such as γH2AX), hypoxia (e.g., using the tracer EF5, either as a metal-containing derivative or coupled to a metal-tagged EF5 specific antibody). As described below, the tissue state may be correlated with the presence and/or distribution of metal containing drugs or accumulated heavy metals.

When detecting metal-containing drugs and/or accumulated heavy metals as described below, the biological sample may be obtained from an animal subject. Specifically, the animal subject may be mammalian (e.g., rodent or human), such as an animal research model or a human patient.

Animal research models include any animal genetically engineered and/or put under conditions (e.g., xenograft of a human tumor, exposure to a carcinogen, or exposure to a toxic heavy metal) to induce a diseased state, such as cancer or heavy metal poisoning. In other embodiments, the biological sample is obtained from a human patient, such as a person having or being tested for a cancer or toxic exposure to heavy metal. In either case, the animal subject may be exposed to a chemotherapeutic drug or heavy metal prior to the biological sample being obtained from the animal subject.

Multiplexed detection of metal tags, as described herein, may be used in pulse chase type experiments. Specifically, exposing a living animal or biological sample to metal containing drugs or toxic heavy metals comprising different metal isotope of the same element at different timepoints can be used to monitor the progression of metal retention and/or clearance. In certain aspects, a treatment or change in exposure may coincide with one or more timepoints.

### Labelling of the Tissue Sample

The disclosure produces samples which have been labelled with labelling atoms, for example a plurality of different labelling atoms, wherein the labelling atoms are detected by an apparatus capable of sampling specific, preferably subcellular, areas of a sample (the labelling atoms therefore represent an elemental tag). The reference to a plurality of different atoms means that more than one atomic species is used to label the sample. These atomic species can be distinguished using a mass detector (e.g. they have different m/Q ratios), such that the presence of two different labelling atoms within a plume gives rise to two different MS signals. The atomic species can also be distinguished using an optical spectrometer (e.g. different atoms have different emission spectra), such that the presence of two different labelling atoms within a plume gives rise to two different emission spectral signals.

The methods herein are suitable for the simultaneous detection of many more than two different labelling atoms, permitting multiplex label detection e.g. at least 3, 4, 5, 10, 20, 30, 32, 40, 50 or even 100 different labelling atoms. Labelling atoms can also be used in a combinatorial manner to even further increase the number of distinguishable labels, if a combination of labelling atoms can be individually resolved. Giesen et al. 2014 demonstrates the use of 32 different labelling atoms in an imaging method, but laser ablation inductively coupled plasma mass spectrometry (LA-ICP-MS) is intrinsically suitable for parallel detection of higher numbers of different atoms e.g. even over 100 different atomic species, as are the other techniques discussed herein. By labelling different targets with different labelling atoms it is possible to determine the cellular location of multiple targets in a single image.

Labelling the tissue sample generally requires that the labelling atoms are attached to one member of a specific binding pair (SBP). This labelled SBP is contacted with a tissue sample such that it can interact with the other member of the SBP (the target SBP member) if it is present, thereby localising the labelling atom to a specific location in the sample. The method of the disclosure then detects the presence of the labelling atom at this specific location and translates this information into an image in which the target SBP member is present at that location. Rare earth metals and other labelling atoms can be conjugated to SBP members by known techniques e.g. Bruckner et al. (2013; Anal. Chem. 86:585-91) describes the attachment of lanthanide atoms to oligonucleotide probes for ICP-MS detection, Gao & Yu (2007; Biosensor Bioelectronics 22:933-40) describes the use of ruthenium to label oligonucleotides, and Fluidigm Canada sells the MaxPar™ metal labelling kits which can be used to conjugate over 30 different labelling atoms to proteins (e.g., antibodies including fragments thereof).

As mentioned above, a mass tag comprising one or more labelling atoms is attached to a SBP member, and this mass-tagged SBP member is contacted with the tissue sample where it can find the target SBP member (if present), thereby forming a labelled target SBP (aka a labelled analyte). The target member can comprise any chemical structure that is suitable for attaching to a labelling atom and then for imaging according to the disclosure.

In general terms, methods of the disclosure can be based on any SBP which is already known for use in determining the location of target molecules in tissue samples (e.g. as used in IHC or fluorescence in situ hybridisation, FISH), but the SBP member which is contacted with the sample will carry a labelling atom which is detectable by a detector system as described above. Thus the disclosure can readily be implemented by using available IHC and FISH reagents, merely by modifying the labels which have previously been used e.g. to modify a FISH probe to carry a label which can be detected.

The common structure of the mass-tagged SBPs resulting from the commonality of the reaction chemistries used to conjugate the SBPs and mass tags can also have advantages in terms of ensuring that the mass tags are ionised comparably to generate elemental ions when different mass-tagged SBPs are deployed together in a multiplexed reaction. Use of a common conjugation chemistry benefits the highly multiplexed analysis uniquely offered by imaging mass cytometry, as different labelling atoms can be more easily attached to different types of SBPs, allowing for a more customizable and flexible assay design. Accordingly, the invention enables the production of labelled samples in which two or more of the mass-tagged SBP reagents have the same linkage between the mass tag and SBP components of the reagent. Accordingly, the invention provides a labelled samples in which two or more of the mass-tagged SBP reagents have the same linkage between the mass tag and SBP components of the reagent. Sometimes, at least 3, at least 4, at least 5, at least 6, at least 7, at least 8, at least 9, at least 10, at least 20, at least 30, at least 40, at least 50, or at least 100 of the mass-tagged SBPs used to stain the stained sample have the same linkage between the mass tag and SBP components of the reagents.

### Target Elements and Detecting the Distribution of Mass (e.g Metal) Tags

Methods may include detecting the distribution of mass (e.g. metal) tags as described herein. In certain aspects, detecting may include constructing an image (as described further herein) that renders the spatial distribution of the mass (e.g. metal) tags.

Certain methods, kits and/or biological samples may include a plurality of mass (e.g. metal) tags, such as 3 or more, 5 or more, 10 or more, 20 or more, 30 or more, 40 or more, or 50 or more metal tags.

In summary of the above in imaging mass spectrometry, the distribution of one or more target elements (i.e., elements or elemental isotopes) may be of interest. In certain aspects, target elements are labelling atoms as described herein. On other instances, the target element may be an atom that is naturally present in the sample, e.g. the target element may be a metal that is naturally coordinated in the active site of certain enzymes. A labelling atom may be directly added to the sample alone or covalently bound to or within a biologically active molecule. In certain embodiments, labelling atoms (e.g., metal tags) may be conjugated to a member of a specific binding pair (SBP), such as an antibody (that binds to its cognate antigen), aptamer or oligonucleotide for hybridizing to a DNA or RNA target, as describe herein. Labelling atoms may be attached to an SBP by any method known in the art. In some embodiments, the labelling atoms are a metal element, such as a lanthanide or transition element or another metal tag as described herein. The metal element may have a mass greater than 60 amu, greater than 80 amu, greater than 100 amu, or greater than 120 amu. Mass spectrometers described herein may deplete elemental ions below the masses of the metal elements, so that abundant lighter elements do not create space-charge effects and/or overwhelm the mass detector.

### Multiplexed Analysis

One feature of the disclosure is its ability to detect multiple (e.g. 10 or more, 20 or more, 30 or more, 40 or more or 50 or more, and even up to 100 or more) different target SBP members in a sample e.g. to detect multiple different proteins and/or multiple different nucleic acid sequences. To permit differential detection of these target SBP members their respective SBP members should carry different labelling atoms such that their signals can be distinguished. For instance, where ten different proteins are being detected, ten different antibodies (each specific for a different target protein) can be used, each of which carries a unique label, such that signals from the different antibodies can be distinguished. In some embodiments, it is desirable to use multiple different antibodies against a single target e.g. which recognise different epitopes on the same protein. Thus, a method may use more antibodies than targets due to redundancy of this type. In general, however, the disclosure will use a plurality of different labelling atoms to detect a plurality of different targets.

If more than one labelled antibody is used with the disclosure, it is preferable that the antibodies should have similar affinities for their respective antigens, as this helps to ensure that the relationship between the quantity of labelling atoms detected and the abundance of the target antigen in the tissue sample will be more consistent across different SBPs (particularly at high scanning frequencies). Similarly, it is preferable if the labelling of the various antibodies has the same efficiency, so that the antibodies each carry a comparable quantity of the labelling atom.

In some instances, the SBP may carry a fluorescent label as well as an elemental tag. Fluorescence of the sample may then be used to determine regions of the sample, e.g. a tissue section, comprising material of interest which can then be sampled for detection of labelling atoms. E.g. a fluorescent label may be conjugated to an antibody which binds to an antigen abundant on cancer cells, and any fluorescent cell may then be targeted to determine expression of other cellular proteins that are about by SBPs conjugated to labelling atoms. Where a SBP carries a fluorescent tag in addition to a mass tag, the fluorescent and mass tags may be conjugated to the SBP by different chemistries. For instance, the mass tag may be conjugated using a click chemistry reaction; and the fluorescent tag may be conjugated by the prior art maleimide chemistry to conjugate the fluorescent tag to a sulfhydryl on the SBP. Alternatively, both the fluorescent and mass tags may be conjugated to the SBP by click chemistry. If a target SBP member is located intracellularly, it will typically be necessary to permeabilize cell membranes before or during contacting of the sample with the labels. For example, when the target is a DNA sequence but the labelled SBP member cannot penetrate the membranes of live cells, the cells of the tissue sample can be fixed and permeabilised. The labelled SBP member can then enter the cell and form a SBP with the target SBP member. In this respect, known protocols for use with IHC and FISH can be utilised.

A method may be used to detect at least one intracellular target and at least one cell surface target. In some embodiments, however, the disclosure can be used to detect a plurality of cell surface targets while ignoring intracellular targets. Overall, the choice of targets will be determined by the information which is desired from the method, as the disclosure will provide an image of the locations of the chosen targets in the sample.

As described further herein, specific binding partners (i.e., affinity reagents) comprising labelling atoms may be used to stain (contact) a biological sample. Suitable specific binging partners include antibodies (including antibody fragments). Labelling atoms may be distinguishable by mass spectrometry (i.e., may have different masses). Labelling atoms may be referred to herein as mass (e.g. metal) tags when they include one or more metal atoms. Mass (e.g. metal) tags may include a polymer with a carbon backbone and a plurality of pendant groups that each bind a metal atom (i.e. metal-chelating groups loaded with a metal atom). Alternatively, or in addition, metal tags may include a metal nanoparticle. Antibodies may be tagged with a mass (e.g. metal) tag by a covalent or non-covalent interaction.

Antibody stains may be used to image proteins at cellular or subcellular resolution. Aspects of the invention include contacting the sample with one or more antibodies that specifically bind a protein expressed by cells of the biological sample, wherein the antibody is tagged with a first mass (e.g. metal) tag. For example, the sample may be contacted with 5 or more, 10 or more, 15 or more, 20 or more, 30 or more, 40 or more, 50 or more antibodies, each with a distinguishable mass (e.g. metal) tag. The sample may further be contacted with one or more histochemical stains before, during (e.g., for ease of workflow), or after (e.g., to avoid altering antigen targets of antibodies) staining the sample with antibodies. The sample may further comprise one or more metal containing drugs and/or accumulated heavy metals as described herein.

Mass- (e.g. metal-) tagged antibodies for use in the subject inventions may specifically bind a metabolic probe that does not comprise a metal (e.g., EF5). Other mass- (e.g. metal-) tagged antibodies may specifically bind a target (e.g., of epithelial tissue, stromal tissue, nucleus, etc.) of traditional stains used in fluorescence and light microscopy. Such antibodies include anti-cadherin, anti-collagen, anti-keratin, anti-EFS, anti-Histone H3 antibodies, and a number of other antibodies known in the art.

Alternatively or in addition, detecting the distribution of mass (e.g. metal) tags may include measuring the extent of colocalization of two or more mass (e.g. metal) tags (e.g., assigning a value to the degree to which mass (e.g. metal) tags occupy the same or similar location). Such analysis can be useful for identifying subcellular structures at which mass (e.g. metal) tags are accumulated, which may inform understanding of the biology of exposure to the mass (e.g. metal) tags (or chemicals containing the mass (e.g. metal) tags). In some embodiments, the detection of the spatial distribution of mass (e.g. metal) tags may be at subcellular resolution. In some embodiments, some or all of the mass (e.g. metal) tags may not be endogenous to the biological sample.

### Single Cell Analysis

Methods of the disclosure include laser ablation of multiple cells in a sample, and thus plumes from multiple cells are analysed and their contents are mapped to specific locations in the sample to provide an image. In most cases a user of the method will need to localise the signals to specific cells within the sample, rather than to the sample as a whole. To achieve this, the boundaries of cells (e.g. the plasma membrane, or in some cases the cell wall) in the sample can be demarcated.

Demarcation of cellular boundaries can be achieved in various ways. For instance, a sample can be studied using conventional techniques which can demarcate cellular boundaries, such as microscopy as discussed above. When performing these methods, therefore, an analysis system comprising a camera as discussed above is particularly useful. An image of this sample can then be prepared using a method of the disclosure, and this image can be superimposed on the earlier results, thereby permitting the detected signals to be localised to specific cells. Indeed, as discussed above, in some cases the laser ablation may be directed only to a subset of cells in the sample as determined to be of interest by the use of microscopy based techniques.

To avoid the need to use multiple techniques, however, it is possible to demarcate cellular boundaries as part of the imaging method of the disclosure. Such boundary demarcation strategies are familiar from IHC and immunocytochemistry, and these approaches can be adapted by using labels which can be detected. For instance, the method can involve labelling of target molecule(s) which are known to be located at cellular boundaries, and signal from these labels can then be used for boundary demarcation. Suitable target molecules include abundant or universal markers of cell boundaries, such as members of adhesion complexes (e.g. β-catenin or E-cadherin). Some embodiments can label more than one membrane protein in order to enhance demarcation.

In addition to demarcating cell boundaries by including suitable labels, it is also possible to demarcate specific organelles in this way. For instance, antigens such as histones (e.g. H3) can be used to identify the nucleus, and it is also possible to label mitochondrial-specific antigens, cytoskeleton-specific antigens, Golgi-specific antigens, ribosome-specific antigens, etc., thereby permitting cellular ultrastructure to be analysed by methods of the disclosure.

Signals which demarcate the boundary of a cell (or an organelle) can be assessed by eye, or can be analysed by computer using image processing. Such techniques are known in the art for other imaging techniques e.g. Arce et al. (2013; Scientific Reports 3, article 2266) describes a segmentation scheme that uses spatial filtering to determine cell boundaries from fluorescence images, reference Ali et al. (2011; Mach Vis Appl 23:607-21) discloses an algorithm which determines boundaries from brightfield microscopy images, reference Pound et al. (2012; The Plant Cell 24:1353-61) discloses the CelISeT method to extract cell geometry from confocal microscope images, and reference Hodneland et al. (2013; Source Code for Biology and Medicine 8:16) discloses the CellSegm MATLAB toolbox for fluorescence microscope images. A method which is useful with the disclosure uses watershed transformation and Gaussian blurring. These image processing techniques can be used on their own, or they can be used and then checked by eye.

Once cellular boundaries have been demarcated it is possible to allocate signal from specific target molecules to individual cells. It can also be possible to quantify the amount of a target analyte(s) in an individual cell e.g. by calibrating the methods against quantitative standards.

### Sample Carrier

In certain embodiments, the sample may be immobilized on a solid support (i.e. a sample carrier), to position it for imaging mass spectrometry. The sample carrier may be optically transparent, for example made of glass or plastic. Where the sample carrier is optically transparent, it enables ablation of the sample material through the support. For example, the solid support may include a tissue slide. Sometimes, the sample carrier will comprise features that act as reference points for use with the apparatus and methods described herein, for instance to allow the calculation of the relative position of features/regions of interest that are to be ablated or desorbed and analysed.

Apparatus and Techniques for Use with the Invention

In general terms, the mass imaging apparatus disclosed herein comprises two broadly characterised systems for performing imaging elemental mass spectrometry.

The first is a sampling and ionisation system. This system contains a sample chamber, which is the component in which the sample is placed when it is subjected to analysis. The sample chamber comprises a stage, which holds the sample (typically the sample is on a sample carrier, such as a microscope slide, e.g. a tissue section, a monolayer of cells or individual cells, such as where a cell suspension has been dropped onto the microscope slide, and the slide is placed on the stage). The sampling and ionisation system acts to remove material from the sample in the sample chamber (the removed material being called sample material herein) which is converted into ions, either as part of the process that causes the removal of the material from the sample or via a separate ionisation system, downstream of the sampling system.

The ionised material is then analysed by the second system which is the detector system. The detector system can take different forms depending upon the particular characteristic of the ionised sample material being determined, for example a mass detector or an optical emission detector in mass spectrometry-based and optical spectrometer-based mass imaging apparatus, respectively.

Thus, in operation, the sample is taken into the apparatus, is sampled to generate ionised material (sampling may generate vaporous/particular material, which is subsequently ionised by the ionisation system), and the ions of the sample material are passed into the detector system. Although the detector system can detect many ions, most of these will be ions of the atoms that naturally make up the sample. In some applications, for example analysis of minerals, such as in geological or archaeological applications, this may be sufficient.

In some cases, for example when analysing biological samples, the native element composition of the sample may not be suitably informative. This is because, typically, all proteins and nucleic acids are comprised of the same main constituent atoms, and so while it is possible to tell regions which contain protein/nucleic acid from those that do not contain such proteinaceous or nucleic acid material, it is not possible to differentiate a particular protein from all other proteins. However, by labelling the sample with atoms not present in the material being analysed under normal conditions, or at least not present in significant amounts (for example certain transition metal atoms, such as rare earth metals; see section on labelling below for further detail), specific characteristics of the sample can be determined. In common with IHC and FISH, the detectable labels can be attached to specific targets on or in the sample (such as fixed cells or a tissue sample on a slide), inter alia through the use of SBPs such as antibodies nucleic acids or lectins, etc. targeting molecules on or in the sample. In order to detect the ionised label, the detector system is used, as it would be to detect ions from atoms naturally present in the sample. By linking the detected signals to the known positions of the sampling of the sample which gave rise to those signals it is possible to generate an image of the atoms present at each position, both the native elemental composition and any labelling atoms. In aspects where native elemental composition of the sample is depleted prior to detection, the image may only be of labelling atoms. The technique allows the analysis of many labels in parallel (also termed multiplexing), which is a great advantage in the analysis of biological samples.

Thus various types of mass imaging apparatus can be used in practicing the disclosure, a number of which are discussed in detail below.

### Mass Imaging Apparatus Based on Mass-Detection

### 1. Sampling and Ionisation Systems

a. Laser Ablation Sampling and Ionising System

A laser ablation based analyser typically comprises three components. The first is a laser ablation sampling system for the generation of plumes of vaporous and particulate material from the sample for analysis. Before the atoms in the plumes of ablated sample material (including any detectable labelling atoms as discussed below) can be detected by the detector system—a mass spectrometer component (MS component; the third component), the sample must be ionised (and atomised). Accordingly, the apparatus comprises a second component which is an ionisation system that ionises the atoms to form elemental ions to enable their detection by the MS component based on mass/charge ratio (some ionisation of the sample material may occur at the point of ablation, but space charge effects result in the almost immediate neutralisation of the charges). The laser ablation sampling system is connected to the ionisation system by a transfer conduit.

### Laser Ablation Sampling System

In brief summary, the components of a laser ablation sampling system include a laser source that emits a beam of laser radiation that is directed upon a sample. The sample is positioned on a stage within a chamber in the laser ablation sampling system (the sample chamber). The stage is usually a translation stage, so that the sample can be moved relative to the beam of laser radiation whereby different locations on the sample can be sampled for analysis. As discussed below in more detail, gas is flowed through the sample chamber, and the flow of gas carries away the plumes of aerosolised material generated when the laser source ablates the sample, for analysis and construction of an image of the sample based on its elemental composition (including labelling atoms such as labelling atoms from elemental tags). As explained further below, in an alternative mode of action, the laser system of the laser ablation sampling system can also be used to desorb material from the sample.

For biological samples (cells, tissues sections etc.) in particular, the sample is often heterogeneous (although heterogeneous samples are known in other fields of application of the disclosure, i.e. samples of a non-biological nature). A heterogeneous sample is a sample containing regions composed of different materials, and so some regions of the sample can ablate at lower threshold fluence at a given wavelength than the others. The factors that affect ablation thresholds are the absorbance coefficient of the material and mechanical strength of material. For biological tissues, the absorbance coefficient will have a dominant effect as it can vary with the laser radiation wavelength by several orders of magnitude. For instance, in a biological sample, when utilising nanosecond laser pulses a region that contains proteinaceous material will absorb more readily in the 200-230 nm wavelength range, while a region containing predominantly DNA will absorb more readily in the 260-280 nm wavelength range.

It is possible to conduct laser ablation at a fluence near the ablation threshold of the sample material. Ablating in this manner often improves aerosol formation which in turn can help improve the quality of the data following analysis. Often to obtain the smallest crater, to maximise the resolution of the resulting image, a Gaussian beam is employed. A cross section across a Gaussian beam records an energy density profile that has a Gaussian distribution. In that case, the fluence of the beam changes with the distance from the centre. As a result, the diameter of the ablation spot size is a function of two parameters: (i) the Gaussian beam waist (1/e²), and (ii) the ratio between the fluence applied and the threshold fluence.

Thus, in order to ensure consistent removal of a reproducible quantity of material with each ablative laser pulse, and thus maximise the quality of the imaging data, it is useful to maintain a consistent ablation diameter which in turn means adjusting the ratio of the energy supplied by the laser pulse to the target to the ablation threshold energy of the material being ablated. This requirement represents a problem when ablating a heterogeneous sample where the threshold ablation energy varies across the sample, such as a biological tissue where the ratio of DNA and protein material varies, or in a geological sample, where it varies with the particular composition of the mineral in the region of the sample. To address this, more than one wavelength of laser radiation can be focused onto the same ablation location on a sample, to more effectively ablate the sample based on the composition of the sample at that location.

### Lasers

Generally, the choice of wavelength and power of the laser used for ablation of the sample can follow normal usage in cellular analysis. The laser must have sufficient fluence to cause ablation to a desired depth, without substantially ablating the sample carrier. A laser fluence of between 0.1-5 J/cm² is typically suitable e.g. from 3-4 J/cm² or about 3.5 J/cm², and the laser will ideally be able to generate a pulse with this fluence at a rate of 200 Hz or greater. In some instances, a single laser pulse from such a laser should be sufficient to ablate cellular material for analysis, such that the laser pulse frequency matches the frequency with which ablation plumes are generated. In general, to be a laser useful for imaging biological samples, the laser should produce a pulse with duration below 100 ns (preferably below 1 ns) which can be focused to, for example, the specific spot sizes discussed below. In some embodiments of the present invention, the ablation rate (i.e. the rate at which the laser ablates a spot on the surface of the sample) is 200 Hz or greater, such as 500 Hz or greater, 750 Hz or greater, 1 kHz or greater, 1.5 kHz or greater, 2 kHz or greater, 2.5 kHz or greater, 3 kHz or greater, 3.5 kHz or greater, 4 kHz or greater, 4.5 kHz or greater, 5 kHz or greater, 10 kHz or greater, 100 kHz or greater, 1 MHz or greater, 10 MHz or greater, or 100 MHz or greater. Many lasers have a repetition rate in excess of the laser ablation frequency, and so appropriate components, such as pulse pickers etc. can be employed to control the rate of ablation as appropriate. Accordingly, in some embodiments, the laser repetition rate is at least 1 kHz, such as at least 10 kHz, at least 100 kHz, at least 1 MHz, at least 10 MHz, around 80 MHz, or at least 100 MHz, optionally wherein the sampling system further comprises a pulse picker, such as wherein the pulse picker is controlled by the control module that also controls the movement of the sample stage. In other instances, multiple closely spaced pulse bursts (for example a train of 3 closely spaced pulses) can be used to ablate one single spot. As an example a 10×10 μm area may be ablated by using 100 bursts of 3 closely spaced pulses in each spot; this can be useful for lasers which have limited ablation depth, for example femtosecond lasers, and can generate a continuous plume of ablated cellular material without losing resolution.

For instance, the frequency of ablation by the laser system is within the range 200 Hz-100 MHz, 200 Hz-10 MHz, 200 Hz-1 MHz, 200 Hz-100 kHz, within the range 500-50 kHz, or within the range 1 kHz-10 kHz.

At these frequencies the instrumentation must be able to analyse the ablated material rapidly enough to avoid substantial signal overlap between consecutive ablations, if it is desired to resolve each ablated plume individually (which as set out below may not necessarily be desired when firing a burst of pulses at a sample). It is preferred that the overlap between signals originating from consecutive plumes is <10% in intensity, more preferably <5%, and ideally <2%. The time required for analysis of a plume will depend on the washout time of the sample chamber (see sample chamber section below), the transit time of the plume aerosol to and through the laser ionisation system, and the time taken to analyse the ionised material. Each laser pulse can be correlated to a pixel on the image of the sample that is subsequently built up, as discussed in more detail below.

In some embodiments, the laser source comprises a laser with a nanosecond pulse duration or an ultrafast laser (pulse duration of 1 ps (10⁻¹² s) or quicker, such as a femtosecond laser. Ultrafast pulse durations provide a number of advantages, because they limit heat diffusion from the ablated zone, and thereby provide more precise and reliable ablation craters, as well as minimising scattering of debris from each ablation event.

In some instances a femtosecond laser is used as the laser source. A femtosecond laser is a laser which emits optical pulses with a duration below 1 ps. The generation of such short pulses often employs the technique of passive mode locking. Femtosecond lasers can be generated using a number of types of laser. Typical durations between 30 fs and 30 ps can be achieved using passively mode-locked solid-state bulk lasers. Similarly, various diode-pumped lasers, e.g. based on neodymium-doped or ytterbium-doped gain media, operate in this regime. Titanium-sapphire lasers with advanced dispersion compensation are even suitable for pulse durations below 10 fs, in extreme cases down to approximately 5 fs. The pulse repetition rate is in most cases between 10 MHz and 500 MHz, though there are low repetition rate versions with repetition rates of a few megahertz for higher pulse energies (available from e.g. Lumentum (CA, USA), Radiantis (Spain), Coherent (CA, USA)). This type of laser can come with an amplifier system which increases the pulse energy

There are also various types of ultrafast fiber lasers, which are also in most cases passively mode-locked, typically offering pulse durations between 50 and 500 fs, and repetition rates between 10 and 100 MHz. Such lasers are commercially available from e.g. NKT Photonics (Denmark; formerly Fianium), Amplitude Systems (France), Laser-Femto (CA, USA). The pulse energy of this type of laser can also be increased by an amplifier, often in the form of an integrated fiber amplifier.

Some mode-locked diode lasers can generate pulses with femtosecond durations. Directly at the laser output, the pulse duration is usually around several hundred femtoseconds (available e.g. from Coherent (CA, USA)).

In some instances, a picosecond laser is used. Many of the types of lasers already discussed in the preceding paragraphs can also be adapted to produce pulses of picosecond range duration. The most common sources are actively or passively mode-locked solid-state bulk lasers, for example a passively mode-locked Nd-doped YAG, glass or vanadate laser. Likewise, picosecond mode-locked lasers and laser diodes are commercially available (e.g. NKT Photonics (Denmark), EKSPLA (Lithuania)).

Nanosecond pulse duration lasers (gain switched and Q switched) can also find utility in particular apparatus set ups (Coherent (CA, USA), Thorlabs (NJ, USA)),

Alternatively, a continuous wave laser may be used, externally modulated to produce nanosecond or shorter duration pulses.

Typically, the laser beam used for ablation in the laser systems discussed herein has a spot size, i.e., at the sampling location, of 100 μm or less, such as 50 μm or less, 25 μm or less, 20 μm or less, 15 μm or less, or 10 μm or less, such as about 3 μm or less, about 2 μm or less, about 1 μm or less, about 500 nm or less, about 250 nm or less. The distance referred to as spot size corresponds to the longest internal dimension of the beam, e.g. for a circular beam it is the beam diameter, for a square beam it corresponds to the length of the diagonal between opposed corners, for a quadrilateral it is the length of the longest diagonal etc. (as noted above, the diameter of a circular beam with a Gaussian distribution is defined as the distance between the points at which the fluence has decreased to 1/e² times the peak fluence). As an alternative to the Gaussian beam, beam shaping and beam masking can be employed to provide the desired ablation spot. For example, in some applications, a square ablation spot with a top hat energy distribution can be useful (i.e. a beam with near uniform fluence as opposed to a Gaussian energy distribution). This arrangement reduces the dependence of the ablation spot size on the ratio between the fluence at the peak of the Gaussian energy distribution and the threshold fluence. Ablation at close to the threshold fluence provides more reliable ablation crater generation and controls debris generation. Accordingly, the laser system may comprise beam masking and/or beam shaping components, such as a diffractive optical element, arranged in a Gaussian beam to re-shame the beam and produce a laser focal spot of uniform or near-uniform fluence, such as a fluence that varies across the beam by less than ±25%, such as less than ±20%, ±15%, ±10% or less than ±5%. Sometimes, the laser beam has a square cross-sectional shape. Sometimes, the beam has a top hat energy distribution.

When used for analysis of biological samples, in order to analyse individual cells the spot size of laser beam used will depend on the size and spacing of the cells. For example, where the cells are tightly packed against one another (such as in a tissue section) one or more laser sources in the laser system can have a spot size which is no larger than these cells. This size will depend on the particular cells in a sample, but in general the laser spot will have a diameter of less than 4 μm e.g. about 3 μm or less, about 2 μm or less, about 1 μm or less, about 500 nm or less, about 250 nm or less, or between 300 nm and 1 μm. In order to analyse given cells at a subcellular resolution the system uses a laser spot size which is no larger than these cells, and more specifically uses a laser spot size which can ablate material with a subcellular resolution. Sometimes, single cell analysis can be performed using a spot size larger than the size of the cell, for example where cells are spread out on the slide, with space between the cells. Here, a larger spot size can be used and single cell characterisation achieved, because the additional ablated area around the cell of interest does not comprise additional cells. The particular spot size used can therefore be selected appropriately dependent upon the size of the cells being analysed. In biological samples, the cells will rarely all be of the same size, and so if subcellular resolution imaging is desired, the ablation spot size should be smaller than the smallest cell, if constant spot size is maintained throughout the ablation procedure. Small spot sizes can be achieved using focusing of laser beams. A laser spot diameter of 1 μm corresponds to a laser focus point (i.e. the diameter of the laser beam at the focal point of the beam) of 1 μm, but the laser focus point can vary by +20% or more due to spatial distribution of energy on the target (for instance, Gaussian beam shape) and variation in total laser energy with respect to the ablation threshold energy. Suitable objectives for focusing a laser beam include a reflecting objective, such as an objective of a Schwarzschild Cassegrain design (reverse Cassegrain). Refracting objectives can also be used, as can combination reflecting-refracting objectives. A single aspheric lens can also be used to achieve the required focusing. A solid-immersion lens or diffractive optic can also be used to focus the laser beam. Another means for controlling the spot size of the laser, which can be used alone or in combination with the above objectives is to pass the beam through an aperture prior to focusing. Different beam diameters can be achieved by passing the beam through apertures of different diameter from an array of diameters. In some instances, there is a single aperture of variable size, for example when the aperture is a diaphragm aperture. Sometimes, the diaphragm aperture is an iris diaphragm. Variation of the spot size can also be achieved through dithering of the optics. The one or more lenses and one or more apertures are positioned between the laser and the sample stage.

For completeness, the standard lasers for LA at sub-cellular resolution, as known in the art (e.g. [5]), are excimer or exciplex lasers. Suitable results can be obtained using an argon fluoride laser (λ=193 nm). Pulse durations of 10-15 ns with these lasers can achieve adequate ablation.

Overall, the laser pulse frequency and strength are selected in combination with the response characteristics of the MS detector to permit distinct detection of individual laser ablation plumes. In combination with using a small laser spot and a sample chamber having a short washout time, rapid and high resolution imaging is now feasible.

### Laser Ablation Focal Point

To maximise the efficiency of a laser to ablate material from a sample, the sample should be at a suitable position with regard to the laser's focal point, for example at the focal point, as the focal point is where the laser beam will have the smallest diameter and so most concentrated energy. This can be achieved in a number of ways. A first way is that the sample can be moved in the axis of the laser light directed upon it (i.e. up and down the path of the laser light/towards and away from the laser source) to the desired point at which the light is of sufficient intensity to effect the desired ablation. Alternatively, or additionally, lenses can be used to move the focal point of the laser light and so its effective ability to ablate material at the location of the sample, for example by demagnification. The one or more lenses are positioned between the laser and the sample stage. A third way, which can be used alone or in combination with either or both of the two preceding ways, is to alter the position of the laser.

To assist the user of the system in placing the sample at the most suitable location for ablation of material from it, a camera can be directed at the stage holding the sample (discussed in more detail below). Accordingly, the disclosure provides a laser ablation sampling system comprising a camera directed on the sample stage. The image detected by the camera can be focussed to the same point at which the laser is focussed. This can be accomplished by using the same objective lens for both laser ablation and optical imaging. By bringing the focal point of two into accordance, the user can be sure that laser ablation will be most effective when the optical image is in focus. Precise movement of the stage to bring the sample into focus can be effected by use of piezo activators, as available from Physik Instrumente, Cedrat-technologies, Thorlabs and other suppliers.

In a further mode of operation, the laser ablation is directed to the sample through the sample carrier. In this instance, the sample support should be chosen so that it is transparent (at least partially) to the frequency of laser radiation being employed to ablate the sample. Ablation through the sample can have advantages in particular situations, because this mode of ablation can impart additional kinetic energy to the plume of material ablated from the sample, driving the ablated material further away from the surface of the sample, so facilitating the ablated material's being transported away from the sample for analysis in the detector. Likewise, desorption based methods which remove slugs of sample material can also be mediated by laser radiation which passes through the carrier. The additional kinetic energy provided to the slug of material being desorbed can assist in catapulting the slug away from the sample carrier, and so facilitating the slug's being entrained in the carrier gas being flowed through the sample chamber.

In order to achieve 3D-imaging of the sample, the sample, or a defined area thereof, can be ablated to a first depth, which is not completely through the sample. Following this, the same area can be ablated again to a second depth, and so on to third, fourth, etc. depths. This way a 3D image of the sample can be built up. In some instances, it may be preferred to ablate all of the area for ablation to a first depth before proceeding to ablate at the second depth. Alternatively, repeated ablation at the same spot may be performed to ablate through different depths before proceeding onto the next location in the area for ablation. In both instances, deconvolution of the resulting signals at the MS to locations and depths of the sample can be performed by the imaging software.

### Laser System Optics for Multiple Modes of Operation

As a matter of routine arrangement, optical components can be used to direct laser radiation, optionally of different wavelengths, to different relative locations. Optical components can also be arranged in order to direct laser radiation, optionally of different wavelengths, onto the sample from different directions. For example one or more wavelengths can be directed onto the sample from above, and one or more wavelengths of laser radiation (optionally different wavelengths) can be directed from below (i.e. through the substrate, such as a microscope slide, which carries the sample, also termed the sample carrier), or indeed the same wavelength can be directed from above and/or below. This enables multiple modes of operation for the same apparatus. Accordingly, the laser system can comprise an arrangement of optical components, arranged to direct laser radiation, optionally of different wavelengths, onto the sample from different directions. Thus optical components may be arranged such that the arrangement directs laser radiation, optionally of different wavelengths, onto the sample from opposite directions. “Opposite” directions in this context is not limited to laser radiation directed perpendicularly onto the sample from above and below (which would be 180° opposite), but includes arrangements which direct laser radiation onto the sample at angles other than perpendicular to the sample. There is no requirement for the laser radiation directed onto the sample from different directions to be parallel. Sometimes, when the sample is on a sample carrier, the reflector arrangement can be arranged to direct laser radiation of a first wavelength directly onto the sample and to direct laser radiation of a second wavelength to the sample through the sample carrier.

Directing laser radiation through the sample carrier to the sample can be used to ablate the sample. In some systems, however, directing the laser radiation through the carrier can be used for “LIFTing” modes of operation, as discussed below in more detail in relation to desorption based sampling systems (although as will be appreciated by one of skill in the art, ablation and LIFTing can be performed by the same apparatus, and so what is termed herein a laser ablation sampling system can also act as a desorption based sampling system). The NA (numerical aperture) of the lens used to focus the laser radiation onto the sample from the first direction may be different from the NA of the lens used to focus the laser radiation (optionally at a different wavelength) onto the sample from the second direction. The lifting operation (e.g. where laser radiation is directed through the sample carrier) often employs a spot size of greater diameter than when ablation is being performed.

### Sample Chamber of the Laser Ablation Sampling System

The sample is placed in the sample chamber when it is subjected to laser ablation. The sample chamber comprises a stage, which holds the sample (typically the sample is on a sample carrier). When ablated, the material in the sample forms plumes, and the flow of gas passed through the sample chamber from a gas inlet to a gas outlet carries away the plumes of aerosolised material, including any labelling atoms that were at the ablated location. The gas carries the material to the ionisation system, which ionises the material to enable detection by the detector. The atoms, including the labelling atoms, in the sample can be distinguished by the detector and so their detection reveals the presence or absence of multiple targets in a plume and so a determination of what targets were present at the ablated locus on the sample. Accordingly, the sample chamber plays a dual role in hosting the solid sample that is analysed, but also in being the starting point of the transfer of aerosolised material to the ionisation and detection systems. This means that the gas flow through the chamber can affect how spread out the ablated plume of material becomes as it passes through the system. A measure of how spread out the ablated plume becomes is the washout time of the sample chamber. This value is a measure of how long it takes material ablated from the sample to be carried out of the sample chamber by the gas flowing through it.

The spatial resolution of the signals generated from laser ablation (i.e. when ablation is used for imaging rather than exclusively for clearing, as discussed below) in this way depends on factors including: (i) the spot size of the laser, as signal is integrated over the total area which is ablated; and the speed with which plumes are generated versus the movement of the sample relative to the laser, and (ii) the speed at which a plume can be analysed, relative to the speed at which plumes are being generated, to avoid overlap of signal from consecutive plumes as mentioned above. Accordingly, being able to analyse a plume in the shortest time possible minimises the likelihood of plume overlap (and so in turn enables plumes to be generated more frequently), if individual analysis of plumes is desired.

Accordingly, a sample chamber with a short washout time (e.g. 100 ms or less) is advantageous for use with the apparatus and methods disclosed herein. A sample chamber with a long washout time will either limit the speed at which an image can be generated or will lead to overlap between signals originating from consecutive sample spots (e.g. Kindness et al. (2003; Clin Chem 49:1916-23), which had signal duration of over 10 seconds). Therefore aerosol washout time is a key limiting factor for achieving high resolution without increasing total scan time. Sample chambers with washout times of ≤100 ms are known in the art. For example, Gurevich & Hergenröder (2007; J. Anal. At. Spectrom., 22:1043-1050) discloses a sample chamber with a washout time below 100 ms. A sample chamber was disclosed in Wang et al. (2013; Anal. Chem. 85:10107-16) (see also WO 2014/146724) which has a washout time of 30 ms or less, thereby permitting a high ablation frequency (e.g. above 20 Hz) and thus rapid analysis. Another such sample chamber is disclosed in WO 2014/127034. The sample chamber in WO 2014/127034 comprises a sample capture cell configured to be arranged operably proximate to the target, the sample capture cell including: a capture cavity having an opening formed in a surface of the capture cell, wherein the capture cavity is configured to receive, through the opening, target material ejected or generated from the laser ablation site and a guide wall exposed within the capture cavity and configured to direct a flow of the carrier gas within the capture cavity from an inlet to an outlet such that at least a portion of the target material received within the capture cavity is transferrable into the outlet as a sample. The volume of the capture cavity in the sample chamber of WO 2014/127034 is less than 1 cm³ and can be below 0.005 cm³. Sometimes the sample chamber has a washout time of 25 ms or less, such as 20 ms, 10 ms or less, 5 ms or less, 2 ms or less, 1 ms, less or 500 μs or less, 200 μs or less, 100 μs or less, 50 μs or less, or 25 μs or less. For example, the sample chamber may have a washout time of 10 μs or more. Typically, the sample chamber has a washout time of 5 ms or less.

For completeness, sometimes the plumes from the sample can be generated more frequently than the washout time of the sample chamber, and the resulting images will smear accordingly (e.g. if the highest possible resolution is not deemed necessary for the particular analysis being undertaken).

The sample chamber typically comprises a translation stage which holds the sample (and sample carrier) and moves the sample relative to the beams of laser radiation. When a mode of operation is used which requires the direction of laser radiation through the sample carrier to the sample, e.g. as in the lifting methods discussed herein, the stage holding the sample carrier should also be transparent to the laser radiation used.

Thus, the sample may be positioned on the side of the sample carrier (e.g., glass slide) facing the laser radiation as it is directed onto the sample, such that ablation plumes are released on, and captured from, the same side as that from which the laser radiation is directed onto the sample. Alternatively, the sample may be positioned on the side of the sample carrier opposite to the laser radiation as it is directed onto the sample (i.e. the laser radiation passes through the sample carrier before reaching the sample), and ablation plumes are released on, and captured from, the opposite side to the laser radiation.

One feature of a sample chamber, which is of particular use where specific portions in various discrete areas of sample are ablated, is a wide range of movement in which the sample can be moved in the x and y (i.e. horizontal) axes in relation to the laser (where the laser beam is directed onto the sample in the z axis), with the x and y axes being perpendicular to one another. More reliable and accurate relative positions are achieved by moving the stage within the sample chamber and keeping the laser's position fixed in the laser ablation sampling system of the apparatus. The greater the range of movement, the more distant the discrete ablated areas can be from one another. The sample is moved in relation to the laser by moving the stage on which the sample is placed. Accordingly, the sample stage can have a range of movement within the sample chamber of at least 10 mm in the x and y axes, such as 20 mm in the x and y axes, 30 mm in the x and y axes, 40 mm in the x and y axes, 50 mm in the x and y axes, such as 75 mm in the x and y axes. Sometimes, the range of movement is such that it permits the entire surface of a standard 25 mm by 75 mm microscope slide to be analysed within the chamber. Of course, to enable subcellular ablation to be achieved, in addition to a wide range of movement, the movement should be precise. Accordingly, the stage can be configured to move the sample in the x and y axes in increments of less than 10 μm, such as less than 5 μm, less than 4 μm, less than 3 μm, less than 2 μm, 1 μm, or less than 1 μm, such as less than 500 nm, less than 200 nm, less than 100 nm. For example, the stage may be configured to move the sample in increments of at least 50 nm. Precise stage movements can be in increments of about 1 μm, such as 1 μm±0.1 μm. Commercially available microscope stages can be used, for example as available from Thorlabs, Prior Scientific, and Applied Scientific Instrumentation. Alternatively, the motorised stage can be built from components, based on positioners providing the desired range of movement and suitably fine precision movement, such as the SLC-24 positioners from Smaract. The movement speed of the sample stage can also affect the speed of the analysis. Accordingly, the sample stage has an operating speed of greater than 1 mm/s, such as 10 mm/s, 50 mm/s or 100 mm/s.

Naturally, when a sample stage in a sample chamber has a wide range of movement, the sample must be sized appropriately to accommodate the movements of the stage. Sizing of the sample chamber is therefore dependent on size of the sample to be involved, which in turn determines the size of the mobile sample stage. Exemplary sizes of sample chamber have an internal chamber of 10×10 cm, 15×15 cm or 20×20 cm. The depth of the chamber may be 3 cm, 4 cm or 5 cm. The skilled person will be able to select appropriate dimensions following the teaching herein. The internal dimensions of the sample chamber for analysing biological samples using a laser ablation sampler must be bigger than the range of movement of the sample stage, for example at least 5 mm, such as at least 10 mm. This is because if the walls of the chamber are too close to the edge of the stage, the flow of the carrier gas passing through the chamber which takes the ablated plumes of material away from the sample and into the ionisation system can become turbulent. Turbulent flow disturbs the ablated plumes, and so instead of remaining as a tight cloud of ablated material, the plume of material begins to spread out after it has been ablated and carried away to the ionisation system of the apparatus. A broader peak of the ablated material has negative effects on the data produced by the ionisation and detection systems because it leads to interference due to peak overlap, and so ultimately, less spatially resolved data, unless the rate of ablation is slowed down to such a rate that it is no longer experimentally of interest.

As noted above, the sample chamber comprises a gas inlet and a gas outlet that takes material to the ionisation system. However, it may contain further ports acting as inlets or outlets to direct the flow of gas in the chamber and/or provide a mix of gases to the chamber, as determined to be appropriate by the skilled artisan for the particular ablative process being undertaken.

### Camera

In addition to identifying the most effective positioning of the sample for laser ablation, the inclusion of a camera (such as a charged coupled device image sensor based (CCD) camera or an active pixel sensor based camera), or any other light detecting means in a laser ablation sampling system enables various further analyses and techniques. A CCD is a means for detecting light and converting it into digital information that can be used to generate an image. In a CCD image sensor, there are a series of capacitors that detect light, and each capacitor represents a pixel on the determined image. These capacitors allow the conversion of incoming photons into electrical charges. The CCD is then used to read out these charges, and the recorded charges can be converted into an image. An active-pixel sensor (APS) is an image sensor consisting of an integrated circuit containing an array of pixel sensors, each pixel containing a photodetector and an active amplifier, e.g. a CMOS sensor.

A camera can be incorporated into any laser ablation sampling system discussed herein, or laser desorption ionization system. The camera can be used to scan the sample to identify cells of particular interest or regions of particular interest (for example cells of a particular morphology), or for fluorescent probes specific for an antigen, or an intracellular or structure. In certain embodiments, the fluorescent probes are histochemical stains or antibodies that also comprise a detectable mass tag. Once such cells have been identified, then laser pulses can be directed at these particular cells to ablate material for analysis, for example in an automated (where the system both identifies and ablates the features/regions(s), such as cells(s), of interest) or semi-automated process (where the user of the system, for example a clinical pathologist, identifies the features/regions(s) of interest, which the system then ablates in an automated fashion). This enables a significant increase in the speed at which analyses can be conducted, because instead of needing to ablate the entire sample to analyse particular cells, the cells of interest can be specifically ablated. This leads to efficiencies in methods of analysing biological samples in terms of the time taken to perform the ablation, but in particular in the time taken to interpret the data from the ablation, in terms of constructing images from it. Constructing images from the data is one of the more time-consuming parts of the imaging procedure, and therefore by minimising the data collected to the data from relevant parts of the sample, the overall speed of analysis is increased.

The camera may record the image from a confocal microscope. Confocal microscopy is a form of optical microscopy that offers a number of advantages, including the ability to reduce interference from background information (light) away from the focal plane. This happens by elimination of out-of-focus light or glare. Confocal microscopy can be used to assess unstained samples for the morphology of the cells, or whether a cell is a discrete cell or part of a clump of cells. Often, the sample is specifically labelled with fluorescent markers (such as by labelled antibodies or by labelled nucleic acids). These fluorescent makers can be used to stain specific cell populations (e.g. expressing certain genes and/or proteins) or specific morphological features on cells (such as the nucleus, or mitochondria) and when illuminated with an appropriate wavelength of light, these regions of the sample are specifically identifiable. Some systems described herein therefore can comprise a laser for exciting fluorophores in the labels used to label the sample. Alternatively, an LED light source can be used for exciting the fluorophores. Non-confocal (e.g. wide field) fluorescent microscopy can also be used to identify certain regions of the biological sample, but with lower resolution than confocal microscopy.

An alternative imaging technique is two-photon excitation microscopy (also referred to as non-linear or multiphoton microscopy). The technique commonly employs near-IR light to excite fluorophores. Two photons of IR light are absorbed for each excitation event. Scattering in the tissue is minimized by IR. Further, due to the multiphoton absorption, the background signal is strongly suppressed. The most commonly used fluorophores have excitation spectra in the 400-500 nm range, whereas the laser used to excite the two-photon fluorescence lies in near-IR range. If the fluorophore absorbs two infrared photons simultaneously, it will absorb enough energy to be raised into the excited state. The fluorophore will then emit a single photon with a wavelength that depends on the type of fluorophore used that can then be detected.

When a laser is used to excite fluorophores for fluorescence microscopy, sometimes this laser is the same laser that generates the laser light used to ablate material from the biological sample, but used at a power that is not sufficient to cause ablation of material from the sample. Sometimes the fluorophores are excited by the wavelength of light that the laser then ablates the sample with. In others, a different wavelength may be used, for example by generating different harmonics of the laser to obtain light of different wavelengths, or exploiting different harmonics generated in a harmonic generation system, discussed above, apart from the harmonics which are used to ablate the sample. For example, if the fourth and/or fifth harmonic of a Nd:YAG laser are used, the fundamental harmonic, or the second to third harmonics, could be used for fluorescence microscopy.

As an example technique combining fluorescence and laser ablation, it is possible to label the nuclei of cells in the biological sample with an antibody or nucleic acid conjugated to a fluorescent moiety. Accordingly, by exciting the fluorescent label and then observing and recording the positions of the fluorescence using a camera, it is possible to direct the ablating laser specifically to the nuclei, or to areas not including nuclear material. The division of the sample into nuclei and cytoplasmic regions will find particular application in field of cytochemistry. By using an image sensor (such as a CCD detector or an active pixel sensor, e.g. a CMOS sensor), it is possible to entirely automate the process of identifying features/regions of interest and then ablating them, by using a control module (such as a computer or a programmed chip) which correlates the location of the fluorescence with the x,y coordinates of the sample and then directs the ablation laser to that location. As part of this process the first image taken by the image sensor may have a low objective lens magnification (low numerical aperture), which permits a large area of the sample to be surveyed. Following this, a switch to an objective with a higher magnification can be used to home in on the particular features of interest that have been determined to fluoresce by higher magnification optical imaging. These features recorded to fluoresce may then be ablated by a laser. Using a lower numerical aperture lens first has the further advantage that the depth of field is increased, thus meaning features buried within the sample may be detected with greater sensitivity than screening with a higher numerical aperture lens from the outset.

In methods and systems in which fluorescent imaging is used, the emission path of fluorescent light from the sample to the camera may include one or more lenses and/or one or more optical filters. By including an optical filter adapted to pass a selected spectral bandwidth from one or more of the fluorescent labels, the system is adapted to handle chromatic aberrations associated with emissions from the fluorescent labels. Chromatic aberrations are the result of the failure of lenses to focus light of different wavelengths to the same focal point. Accordingly, by including an optical filter, the background in the optical system is reduced, and the resulting optical image is of higher resolution. A further way to minimise the amount of emitted light of undesired wavelengths that reaches the camera is to exploit chromatic aberration of lenses deliberately by using a series of lenses designed for the transmission and focus of light at the wavelength transmitted by the optical filter, akin to the system explained in WO 2005/121864.

A higher resolution optical image is advantageous in this coupling of optical techniques and laser ablation sampling, because the accuracy of the optical image then determines the precision with which the ablating laser can be directed to ablate the sample.

Accordingly, in some embodiments disclosed herein, the apparatus of the invention comprises a camera. This camera can be used on-line to identify features/areas of the sample, e.g. specific cells, which can then be ablated (or desorbed by LIFTing—see below)

In a further mode of operation combining both fluorescence analysis and laser ablation sampling, instead of analysing the entire slide for fluorescence before targeting laser ablation to those locations, it is possible to fire a pulse from the laser at a spot on the sample (at low energy so as only to excite the fluorescent moieties in the sample rather than ablate the sample) and if a fluorescent emission of expected wavelength is detected, then the sample at the spot can be ablated by firing the laser at that spot at full energy, and the resulting plume analysed by a detector as described below. This has the advantage that the rastering mode of analysis is maintained, but the speed is increased, because it is possible to pulse and test for fluorescence and obtain results immediately from the fluorescence (rather than the time taken to analyse and interpret ion data from the detector to determine if the region was of interest), again enabling only the loci of importance to be targeted for analysis. Accordingly, applying this strategy in imaging a biological sample comprising a plurality of cells, the following steps can be performed: (i) labelling a plurality of different target molecules in the sample with one or more different labelling atoms and one or more fluorescent labels, to provide a labelled sample; (ii) illuminating a known location of the sample with light to excite the one or more fluorescent labels; (iii) observing and recording whether there is fluorescence at the location; (iv) if there is fluorescence, directing laser ablation at the location, to form a plume; (v) subjecting the plume to inductively coupled plasma mass spectrometry, and (vi) repeating steps (ii)-(v) for one or more further known locations on the sample, whereby detection of labelling atoms in the plumes permits construction of an image of the sample of the areas which have been ablated.

In some instances, the sample, or the sample carrier, may be modified so as to contain optically detectable (e.g., by optical or fluorescent microscopy) moieties at specific locations. The fluorescent locations can then be used to positionally orient the sample in the apparatus. The use of such marker locations finds utility, for example, where the sample may have been examined visually “offline”—i.e. in a piece of apparatus other than the apparatus of the invention. Such an optical image can be marked with feature(s)/region(s) of interest, corresponding to particular cells by, say, a physician, before the optical image with the feature(s)/region(s) of interest highlighted and the sample are transferred to an apparatus according to the invention. Here, by reference to the marker locations in the annotated optical image, the apparatus of the invention can identify the corresponding fluorescent positions by use of the camera and calculate an ablative and/or desorptive (LIFTing) plan for the positions of the laser pulses accordingly. Accordingly, in some embodiments, the invention comprises an orientation controller module capable of performing the above steps.

In some instances, selection of the features/regions of interest may performed using the apparatus of the invention, based on an image of the sample taken by the camera of the apparatus of the invention.

### Transfer Conduit

The transfer conduit forms a link between the laser ablation sampling system and the ionisation system, and allows the transportation of plumes of sample material, generated by the laser ablation of the sample, from the laser ablation sampling system to the ionisation system. Part (or all) of the transfer conduit may be formed, for example, by drilling through a suitable material to produce a lumen (e.g., a lumen with a circular, rectangular or other cross-section) for transit of the plume. The transfer conduit sometimes has an inner diameter in the range 0.2 mm to 3 mm. Sometimes, the internal diameter of the transfer conduit can be varied along its length. For example, the transfer conduit may be tapered at an end. A transfer conduit sometimes has a length in the range of 1 centimeter to 100 centimeters. Sometimes the length is no more than 10 centimeters (e.g., 1-10 centimeters), no more than 5 centimeters (e.g., 1-5 centimeters), or no more than 3 cm (e.g., 0.1-3 centimeters). Sometimes the transfer conduit lumen is straight along the entire distance, or nearly the entire distance, from the ablation system to the ionisation system. Other times the transfer conduit lumen is not straight for the entire distance and changes orientation. For example, the transfer conduit may make a gradual 90 degree turn. This configuration allows for the plume generated by ablation of a sample in the laser ablation sampling system to move in a vertical plane initially while the axis at the transfer conduit inlet will be pointing straight up, and move horizontally as it approaches the ionisation system (e.g. an ICP torch which is commonly oriented horizontally to take advantage of convectional cooling). The transfer conduit can be straight for a distance of least 0.1 centimeters, at least 0.5 centimeters or at least 1 centimeter from the inlet aperture though which the plume enters or is formed. In general terms, typically, the transfer conduit is adapted to minimize the time it takes to transfer material from the laser ablation sampling system to the ionisation system.

### Transfer Conduit Inlet, Including Sample Cone

The transfer conduit comprises an inlet in the laser ablation sampling system (in particular within the sample chamber of the laser ablation sampling system; it therefore also represents the principal gas outlet of the sample chamber). The inlet of the transfer conduit receives sample material ablated from a sample in the laser ablation sampling system, and transfers it to the ionisation system. In some instances, the laser ablation sampling system inlet is the source of all gas flow along the transfer conduit to the ionisation system. In some instances, the laser ablation sampling system inlet that receives material from the laser ablation sampling system is an aperture in the wall of a conduit along which a second “transfer” gas is flowed (as disclosed, for example in WO2014146724 and WO2014147260) from a separate transfer flow inlet. In this instance, the transfer gas forms a significant proportion, and in many instances the majority of the gas flow to the ionisation system. The sample chamber of the laser ablation sampling system contains a gas inlet. Flowing gas into the chamber through this inlet creates a flow of gas out of the chamber though the inlet of the transfer conduit. This flow of gas captures plumes of ablated material, and entrains it as it into the transfer conduit (typically the laser ablation sampling system inlet of the transfer conduit is in the shape of a cone, termed herein the sample cone) and out of the sample chamber into the conduit passing above the chamber. This conduit also has gas flowing into it from the separate transfer flow inlet. The component comprising the transfer flow inlet, laser ablation sampling system inlet and which begins the transfer conduit which carries the ablated sample material towards the ionisation system can also termed a flow cell (as it is in WO2014146724 and WO2014147260).

The transfer flow fulfils at least three roles: it flushes the plume entering the transfer conduit in the direction of the ionisation system, and prevents the plume material from contacting the side walls of the transfer conduit; it forms a “protection region” above the sample surface and ensures that the ablation is carried out under a controlled atmosphere; and it increases the flow speed in the transfer conduit. Usually, the viscosity of the capture gas is lower than the viscosity of the primary transfer gas. This helps to confine the plume of sample material in the capture gas in the center of the transfer conduit and to minimize the diffusion of the plume of sample material downstream of the laser ablation sampling system (because in the center of the flow, the transport rate is more constant and nearly flat). The gas(es) may be, for example, and without limitation, argon, xenon, helium, nitrogen, or mixtures of these. A common transfer gas is argon. Argon is particularly well-suited for stopping the diffusion of the plume before it reaches the walls of the transfer conduit (and it also assists improved instrumental sensitivity in apparatus where the ionisation system is an argon gas-based ICP). The capture gas is preferably helium. However, the capture gas may be replaced by or contain other gases, e.g., hydrogen, nitrogen, or water vapor. At 25° C., argon has a viscosity of 22.6 μPas, whereas helium has a viscosity of 19.8 μPas. Sometimes, the capture gas is helium and the transfer gas is argon.

As described in WO2014169394, the use of a sample cone minimizes the distance between the target and the laser ablation sampling system inlet of the transfer conduit. Because of the reduced distance between the sample and the point of the cone through which the capture gas can flow cone, this leads to improved capture of sample material with less turbulence, and so reduced spreading of the plumes of ablated sample material. The inlet of the transfer conduit is therefore the aperture at the tip of the sample cone. The cone projects into the sample chamber.

An optional modification of the sample cone is to make it asymmetrical. When the cone is symmetrical, then right at the center the gas flow from all directions neutralizes, so the overall flow of gas is zero along the surface of the sample at the axis of the sample cone. By making the cone asymmetrical, a non-zero velocity along the sample surface is created, which assists in the washout of plume materials from the sample chamber of the laser ablation sampling system.

In practice, any modification of the sample cone that causes a non-zero vector gas flow along the surface of the sample at the axis of the cone may be employed. For instance, the asymmetric cone may comprise a notch or a series of notches, adapted to generate non-zero vector gas flow along the surface of the sample at the axis of the cone. The asymmetric cone may comprise an orifice in the side of the cone, adapted to generate non-zero vector gas flow along the surface of the sample at the axis of the cone. This orifice will imbalance gas flows around the cone, thereby again generating a non-zero vector gas flow along the surface of the sample at the axis of the cone at the target. The side of the cone may comprise more than one orifice and may include both one or more notches and one or more orifices. The edges of the notch(es) and/or orifice(s) are typically smoothed, rounded or chamfered in order to prevent or minimize turbulence.

Different orientations of the asymmetry of the cone will be appropriate for different situations, dependent on the choice of capture and transfer gas and flow rates thereof, and it is within the abilities of the skilled person to appropriately identify the combinations of gas and flow rate for each orientation.

All of the above adaptations may be present in a single asymmetric sample cone as use in the invention. For example, the cone may be asymmetrically truncated and formed from two different elliptical cone halves, the cone may be asymmetrically truncated and comprise one of more orifices and so on.

The sample cone is therefore adapted to capture a plume of material ablated from a sample in the laser ablation sampling system. In use, the sample cone is positioned operably proximate to the sample, e.g. by manoeuvring the sample within the laser ablation sampling system on a movable sample carrier tray, as described already above. As noted above, plumes of ablated sample material enter the transfer conduit through an aperture at the narrow end of the sample cone. The diameter of the aperture can be a) adjustable; b) sized to prevent perturbation to the ablated plume as it passes into the transfer conduit; and/or c) about the equal to the cross-sectional diameter of the ablated plume.

### Tapered Conduits

In tubes with a smaller internal diameter, the same flow rate of gas moves at a higher speed. Accordingly, by using a tube with a smaller internal diameter, a plume of ablated sample material carried in the gas flow can be transported across a defined distance more rapidly at a given flow rate (e.g. from the laser ablation sampling system to the ionisation system in the transfer conduit). One of the key factors in how quickly an individual plume can be analysed is how much the plume has diffused during the time from its generation by ablation through to the time its component ions are detected at the mass spectrometer component of the apparatus (the transience time at the detector). Accordingly, by using a narrow transfer conduit, the time between ablation and detection is reduced, thereby meaning diffusion is decreased because there is less time in which it can occur, with the ultimate result that the transience time of each ablation plume at the detector is reduced. Lower transience times mean that more plumes can be generated and analyzed per unit time, thus producing images of higher quality and/or faster.

The taper may comprise a gradual change in the internal diameter of the transfer conduit along said portion of the length of the transfer conduit (i.e. the internal diameter of the tube were a cross section taken through it decreases along the portion from the end of the portion towards the inlet (at the laser ablation sampling system end) to the outlet (at the ionisation system end). Usually, the region of the conduit near where ablation occurs has a relatively wide internal diameter. The larger volume of the conduit before the taper facilitates the confinement of the materials generated by ablation. When the ablated particles fly off from the ablated spot they travel at high velocities. The friction in the gas slows these particles down but the plume can still spread on a sub-millimeter to a millimeter scale. Allowing for sufficient distances to the walls helps with the containment of the plume near the center of the flow.

Because the wide internal diameter section is only short (of the order of 1-2 mm), it does not contribute significantly to the overall transience time providing the plume spends more time in the longer portion of the transfer conduit with a narrower internal diameter. Thus, a larger internal diameter portion is used to capture the ablation product and a smaller internal diameter conduit is used to transport these particles rapidly to the ionisation system.

The diameter of the narrow internal diameter section is limited by the diameter corresponding to the onset of turbulence. A Reynolds number can be calculated for a round tube and a known flow. In general a Reynolds number above 4000 will indicate a turbulent flow, and thus should be avoided. A Reynolds number above 2000 will indicate a transitional flow (between non-turbulent and turbulent flow), and thus may also be desired to be avoided. For a given mass flow of gas the Reynolds number is inversely proportional to the diameter of the conduit. The internal diameter of the narrow internal diameter section of the transfer conduit commonly is narrower than 2 mm, for example narrower than 1.5 mm, narrower than 1.25 mm, narrower than 1 mm, but greater than the diameter at which a flow of helium at 4 liters per minute in the conduit has a Reynolds number greater than 4000.

Rough or even angular edges in the transitions between the constant diameter portions of the transfer conduit and the taper may cause turbulence in the gas flow, and typically are avoided.

### Sacrificial Flow

At higher flows, the risk of turbulence occurring in the conduit increases. This is particularly the case where the transfer conduit has a small internal diameter (e.g. 1 mm). However, it is possible to achieve high speed transfer (up to and in excess of 300 m/s) in transfer conduits with a small internal diameter if a light gas, such as helium or hydrogen, is used instead of argon which is traditionally used as the transfer flow of gas.

High speed transfer presents problems insofar as it may cause the plumes of ablated sample material to be passed through the ionisation system without an acceptable level of ionisation occurring. The level of ionisation can drop because the increased flow of cool gas reduces the temperature of the plasma at the end of the torch. If a plume of sample material is not ionised to a suitable level, information is lost from the ablated sample material—because its components (including any labelling atoms/elemental tags) cannot be detected by the mass spectrometer. For example, the sample may pass so quickly through the plasma at the end of the torch in an ICP ionisation system that the plasma ions do not have sufficient time to act on the sample material to ionise it. This problem, caused by high flow, high speed transfer in narrow internal diameter transfer conduits can be solved by the introduction of a flow sacrificing system at the outlet of the transfer conduit. The flow sacrificing system is adapted to receive the flow of gas from the transfer conduit, and pass only a portion of that flow (the central portion of the flow comprising any plumes of ablated sample material) onwards into the injector that leads to the ionisation system. To facilitate dispersion of gas from the transfer conduit in the flow sacrificing system, the transfer conduit outlet can be flared out.

The flow sacrificing system is positioned close to the ionisation system, so that the length of the tube (e.g. injector) that leads from the flow sacrificing system to the ionisation system is short (e.g. ˜1 cm long; compared to the length of the transfer conduit which is usually of a length of the order of tens of cm, such as ˜50 cm). Thus the lower gas velocity within the tube leading from the flow sacrificing system to the ionisation system does not significantly affect the total transfer time, as the relatively slower portion of the overall transport system is much shorter.

In most arrangements, it is not desirable, or in some cases possible, to significantly increase the diameter of the tube (e.g. the injector) which passes from the flow sacrificing system to the ionisation system as a way of reducing the speed of the gas at a volumetric flow rate. For example, where the ionisation system is an ICP, the conduit from the flow sacrificing system forms the injector tube in the center of the ICP torch. When a wider internal diameter injector is used, there is a reduction in signal quality, because the plumes of ablated sample material cannot be injected so precisely into the center of the plasma (which is the hottest and so the most efficiently ionising part of the plasma). The strong preference is for injectors of 1 mm internal diameter, or even narrower (e.g. an internal diameter of 800 μm or less, such as 600 μm or less, 500 μm or less or 400 μm or less). Other ionisation techniques rely on the material to be ionised within a relatively small volume in three dimensional space (because the necessary energy density for ionisation can only be achieved in a small volume), and so a conduit with a wider internal diameter means that much of the sample material passing through the conduit is outside of the zone in which energy density is sufficient to ionise the sample material. Thus narrow diameter tubes from the flow sacrificing system into the ionisation system are also employed in apparatus with non-ICP ionisation systems. As noted above, if a plume of sample material is not ionised to a suitable level, information is lost from the ablated sample material—because its components (including any labelling atoms/elemental tags) cannot be detected by the mass spectrometer.

Pumping can be used to help ensure a desired split ratio between the sacrificial flow and the flow passing into the inlet of the ionisation system. Accordingly, sometimes, the flow sacrificing system comprises a pump attached to the sacrificial flow outlet. A controlled restrictor can be added to the pump to control the sacrificial flow. Sometimes, the flow sacrificing system also comprises a mass flow controller, adapted to control the restrictor.

Where expensive gases are used, the gas pumped out of the sacrificial flow outlet can be cleaned up and recycled back into the same system using known methods of gas purification. Helium is particularly suited as a transport gas as noted above, but it is expensive; thus, it is advantageous to reduce the loss of helium in the system (i.e. when it is passed into the ionisation system and ionised). Accordingly, sometimes a gas purification system is connected to the sacrificial flow outlet of the flow sacrificing system.

### Ionisation System

In order to generate elemental ions, it is necessary to use a hard ionisation technique that is capable of vaporising, atomising and ionising the atomised sample.

### Inductively Coupled Plasma Torch

Commonly, an inductively coupled plasma is used to ionise the material to be analysed before it is passed to the mass detector for analysis. It is a plasma source in which the energy is supplied by electric currents produced by electromagnetic induction. The inductively coupled plasma is sustained in a torch that consists of three concentric tubes, the innermost tube being known as the injector.

The induction coil that provides the electromagnetic energy that maintains the plasma is located around the output end of the torch. The alternating electromagnetic field reverses polarity many millions of times per second. Argon gas is supplied between the two outermost concentric tubes. Free electrons are introduced through an electrical discharge and are then accelerated in the alternating electromagnetic field whereupon they collide with the argon atoms and ionise them. At steady state, the plasma consists of mostly of argon atoms with a small fraction of free electrons and argon ions.

The ICP can be retained in the torch because the flow of gas between the two outermost tubes keeps the plasma away from the walls of the torch. A second flow of argon introduced between the injector (the central tube) and the intermediate tube keeps the plasma clear of the injector. A third flow of gas is introduced into the injector in the centre of the torch. Samples to be analysed are introduced through the injector into the plasma.

The ICP can comprise an injector with an internal diameter of less than 2 mm and more than 250 μm for introducing material from the sample into the plasma. The diameter of the injector refers to the internal diameter of the injector at the end proximal to the plasma. Extending away from the plasma, the injector may be of a different diameter, for example a wider diameter, wherein the difference in diameter is achieved through a stepped increase in diameter or because the injector is tapered along its length. For instance, the internal diameter of the injector can be between 1.75 mm and 250 μm, such as between 1.5 mm and 300 μm in diameter, between 1.25 mm and 300 μm in diameter, between 1 mm and 300 μm in diameter, between 900 μm and 300 μm in diameter, between 900 μm and 400 μm in diameter, for example around 850 μm in diameter. The use of an injector with an internal diameter less than 2 mm provides significant advantages over injectors with a larger diameter. One advantage of this feature is that the transience of the signal detected in the mass detector when a plume of sample material is introduced into the plasma is reduced with a narrower injector (the plume of sample material being the cloud of particular and vaporous material removed from the sample by the laser ablation sampling system). Accordingly, the time taken to analyse a plume of sample material from its introduction into the ICP for ionisation until the detection of the resulting ions in the mass detector is reduced. This decrease in time taken to analyse a plume of sample material enables more plumes of sample material to be detected in any given time period. Also, an injector with a smaller internal diameter results in the more accurate introduction of sample material into the centre of the induction coupled plasma, where more efficient ionisation occurs (in contrast to a larger diameter injector which could introduce sample material more towards the fringe of the plasma, where ionisation is not as efficient).

ICP torches (Agilent, Varian, Nu Instruments, Spectro, Leeman Labs, PerkinElmer, Thermo Fisher etc.) and injectors (for example from Elemental Scientific and Meinhard) are available.

### Other Ionisation Techniques

### Electron Ionisation

Electron ionisation involves bombarding a gas-phase sample with a beam of electrons. An electron ionisation chamber includes a source of electrons and an electron trap. A typical source of the beam of electrons is a rhenium or tungsten wire, usually operated at 70 electron volts energy. Electron beam sources for electron ionisation are available from Markes International. The beam of electrons is directed towards the electron trap, and a magnetic field applied parallel to the direction of the electrons travel causes the electrons to travel in a helical path. The gas-phase sample is directed through the electron ionisation chamber and interacts with the beam of electrons to form ions. Electron ionisation is considered a hard method of ionisation since the process typically causes the sample molecules to fragment. Examples of commercially available electron ionisation systems include the Advanced Markus Electron Ionisation Chamber.

### Optional Further Components of the Laser Ablation Based Sampling and Ionisation System

### Ion Deflector

Mass spectrometers detect ions when they hit a surface of their detector. The collision of an ion with the detector causes the release of electrons from the detector surface. These electrons are multiplied as they pass through the detector (the first released electron knocks out further electrons in the detector, these electrons then hit secondary plates which further amplify the number of electrons). The number of electrons hitting the anode of the detector generates a current. The number of electrons hitting the anode can be controlled by altering the voltage applied to the secondary plates. The current is an analog signal that can then be converted into a count of the ions hitting the detector by an analog-digital converter. When the detector is operating in its linear range, the current can be directly correlated to the number of ions. The quantity of ions that can be detected at once has a limit (which can be expressed as the number of ions detectable per second). Above this point, the number electrons released by ions hitting the detector is no longer correlated to the number of ions. This therefore places an upper limit on the quantitative capabilities of the detector.

When ions hit the detector, its surface becomes damaged by contamination. Over time, this irreversible contamination damage results in fewer electrons being released by the detector surface when an ion hits the detector, with the ultimate result that the detector needs replacing. This is termed “detector aging”, and is a well-known phenomenon in MS.

Detector life can therefore be lengthened by avoiding the introduction of overloading quantities of ions into the MS. As noted above, when the total number of ions hitting the MS detector exceeds the upper limit of detection, the signal is not as informative as when the number of ions is below the upper limit because it is no longer quantitative. It is therefore desirable to avoid exceeding the upper limit of detection as it results in accelerated detector aging without generating useful data.

Analysis of large packets of ions by mass spectrometry involves a particular set of challenges not found in normal mass spectrometry. In particular, typical MS techniques involve introducing a low and constant level of material into the detector, which should not approach the upper detection limit or cause accelerated aging of the detector. On the other hand, laser ablation- and desorption-based techniques analyse a relatively large amount of material in a very short time window in the MS: e.g. the ions from a cell-sized patch of a tissue sample which is much larger than the small packets of ions typically analysed in MS. In effect, it is a deliberate almost overloading of the detector with analysed packed of ions resulting from ablation or lifting. In between the analysis events the signal is at baseline (a signal that is close to zero because no ions from labelling atoms are deliberately being entering into the MS from the sampling and ionisation system; some ions will inevitably be detected because the MS is not a complete vacuum).

Thus in apparatus described herein, there is an elevated risk of accelerated detector aging, because the ions from packets of ionised sample material labelled with a large number of detectable atoms can exceed the upper limit of detection and damage the detector without providing useful data.

To address these issues, the apparatus can comprise an ion deflector positioned between the sampling and ionisation system and the detector system (a mass spectrometer), operable to control the entry of ions into the mass spectrometer. In one arrangement, when the ion deflector is on, the ions received from the sampling and ionisation system are deflected (i.e. the path of the ions is changed and so they do not reach the detector), but when the deflector is off the ions are not deflected and reach the detector. How the ion deflector is deployed will depend on the arrangement of the sampling and ionisation system and MS of the apparatus. E.g. if the portal through which the ions enter the MS is not directly in line with the path of ions exiting the sampling and ionisation system, then by default the appropriately arranged ion deflector will be on, in order to direct ions from the sampling and ionisation system into the MS. When an event resulting from the ionisation a packet of ionised sample material considered likely to overload the MS is detected (see below), the ion deflector is switched off, so that the rest of the ionised material from the event is not deflected into the MS and can instead simply hit an internal surface of the system, thereby preserving the life of the MS detector. The ion deflector is returned to its original state after the ions from the damaging event have been prevented from entering the MS, thereby allowing the ions from subsequent packets of ionised sample material to enter the MS and be detected.

Alternatively, in arrangements where (under normal operating conditions) there is no change in the direction of the ions emerging from the sampling and ionisation system before they enter the MS the ion deflector will be off, and the ions from the sampling and ionisation system will pass through it to be analysed in the MS. To prevent damage when a potential overload of the detector is detected, in this configuration the ion deflector is turned on, and so diverts ions so that they do not enter the detector in order to prevent damage to the detector.

The ions entering the MS from ionisation of sample material (such as a plume of material generated by laser ablation or desorption) do not enter the MS all at the same time, but instead enter as a peak with a frequency that follows a probability distribution curve about a maximum frequency: from baseline, at first a small number of ions enters the MS and are detected, and then the frequency of ions increases to a maximum before the number decreases again and trails off to baseline. An event likely to damage the detector can be identified because instead of a slow increase in the frequency of ions at the leading edge of the peak, there is a very quick increase in counts of ions hitting the detector.

The flow of ions hitting the detector of a TOF MS, a particular type of detector as discussed below, is not continual during the analysis of the ions in a packet of ionised sample material. The TOF comprises a pulser which releases the ions periodically into the flight chamber of the TOF MS in pulsed groups. By releasing the ions all at the known same time, the time of flight mass determination is enabled. The time between the releases of pulses of ions for time of flight mass determination is known as an extraction or push of the TOF MS. The push is in the order of microseconds. The signal from one or more packets of ions from the sampling and ionisation system therefore covers a number of pushes.

Accordingly, when the ion count reading jumps from the baseline to a very high count within one push (i.e. the first portion of the ions from a particular packet of ionised sample material) then it can be predicted that the main body of ions resulting from ionisation of the packet of sample material will be even greater, and so exceed the upper detection limit. It is at this point that an ion deflector can be operated to ensure that the damaging bulk of the ions are directed away from the detector (by being activated or deactivated, depending on the arrangement of the system, as discussed above). Suitable ion deflectors based on quadrupoles are available in the art (e.g. from Colutron Research Corporation and Dreebit GmbH).

b. Desorption Based Sampling and Ionising System

A desorption based analyser typically comprises three components. The first is a desorption system for the generation of slugs of sample material from the sample for analysis. Before the atoms in the slugs of desorbed sample material (including any detectable labelling atoms as discussed below) can be detected, the sample must be ionised (and atomised). Accordingly, the apparatus comprises a second component which is an ionisation system that ionises the atoms to form elemental ions to enable their detection by the MS detector component (third component) based on mass/charge ratio. The desorption based sampling system and the ionisation system are connected by a transfer conduit. In many instances the desorption based sampling system is also a laser ablation based sampling system.

### Desorption Sampling System

In some instances, rather than laser ablation being used to generate a particulate and/or vaporised plume of sample material, a bulk mass of sample material is desorbed from the sample carrier on which it is located without substantial disintegration of the sample and its conversion into small particles and/or vaporisation (see e.g. FIG. 8 of WO2016109825, and the accompanying description, which are herein incorporated by reference). Herein, the term slug is used to refer to this desorbed material (one particular form of a packet of sample material discussed herein). The slug can have dimensions from 10 nm to 10 μm, from 100 nm to 10 μm, and in certain instances from 1 μm to 100 μm. This process can be termed sample catapulting. Commonly, the slug represents a single cell (in which case the process can be termed cell catapulting).

The slug of sample material released from the sample can be a portion of the sample which has been cut into individual slugs for desorption prior to the desorption step, optionally in a process prior to the sample being inserted into the apparatus. A sample divided into discrete slugs prior to analysis is called a structured sample. Each of these individual slugs therefore represents a discrete portion of the sample that can be desorbed, ionised and analysed in the apparatus. By analysis of slugs from the discrete sites, an image can be built up with each slug representing a pixel of the image, in the same way that each location of a sample ablated by the laser ablation sampling system described above.

A structured sample may be prepared by various methods. For instance, a sample carrier comprising topographic features configured to cut a biological sample may be used. Here, a biological sample is applied onto the surface of the carrier, which causes the topographic features to cut and section the sample, in turn causing the sections of biological material to be retained by the plurality of discrete sites between the features to provide a structured biological sample. Alternatively, the sample carrier may not comprise such topographical features (in effect, a flat surface like a microscope slide, optionally functionalised as discussed below), in which case the sample may be applied to the sample carrier and the sample may be sectioned to define slugs of sample that can be desorbed for ionisation and analysis. The sectioning of the sample can be accomplished by mechanical tools such as blades or stamps, if the sample is a tissue section. Alternatively, the material around the sections of the sample to be desorbed can be removed by laser ablation in the same or a separate sample preparation setup. In certain techniques, the material can be removed by a setup employing a focused electron or ion beam. The focused electron or ion beam can lead to particularly narrow cuts (potentially on the 10 nm scale) between subsections leading to a pixel size on the order of 1 μm or in certain instances, 100 nm.

The slugs of sample material can be released from the carrier and each discrete portion of sample material sequentially introduced into the detector for analysis as a discrete event (generating a pixel of an image by the techniques discussed below). The benefits of sequential introduction of discrete material as opposed to random introduction of biological samples as in conventional mass cytometry or mass spectrometry include a higher sample processing rate. This is because the slug is transported from the sample chamber to the ionisation system as preferably a single piece of matter, and so cannot spread out as a plume of ablated material would in a flow of gas (in particular a gas flow in which there is some turbulence).

### Desorption for Sampling

Sample material can be desorbed from the sample by thermal energy, mechanical energy, kinetic energy, and a combination of any of the foregoing. This kind of sampling is useful in particular in analysing biological samples.

In certain instances, sample material may be released from the sample by thermal mechanisms. For example, the surface of sample carrier becomes sufficiently hot to desorb a slug of sample material. The sample carrier may be coated to facilitate the bulk desorption process, for example with polyethylene naphthalate (PEN) polymer or PMMA polymer film. Heat can be provided by a radiative source such as a laser (such as the laser of a laser ablation sampling system discussed above). The energy applied to the surface should be sufficient to desorb the biological material, preferably without altering the sample material if it is from a biological sample. Any suitable radiation wavelength can be used, which can depend in part on the absorptive properties of the sample carrier. A surface or layer of the sample carrier may be coated with or include an absorber that absorbs laser radiation for conversion to heat. The radiation may be delivered to a surface of the carrier other than the surface on which the sample is located, or it may be delivered to the surface carrying the sample, such as through the thickness of the carrier. The heated surface may be a surface layer or may be an inner layer of a multilayer structure of the sample carrier. One example of the use of laser radiation energy is in a technique called lifting (laser induced forward transfer; see e.g. Doraiswamy et al., 2006, Applied Surface Science, 52: 4743-4747; Fernandez-Pradas, 2004, Thin Solid Films 453-454: 27-30; Kyrkis et al., in Recent Advances in Laser Processing of Materials, Eds. Perriere et al., 2006, Elsivier), in which the sample carrier may comprise a desorption film layer. The desorption film can absorb the radiation to cause release of the desorption film and/or the biological sample (e.g. in some instances the sample film desorbs from the sample carrier together with the biological sample, in other instances, the film remains attached to the sample carrier, and the biological sample desorbs from the desorption film).

Desorption by heating can take place on a nanosecond, picosecond or femtosecond time scale, depending on the laser used for desorption.

The sample can be desorbed by the action of a layer of an electrical conductor that heats up upon the application of a current. In such the sites from which sample material is desorbed are electrically connected to electrodes and the sites are individually addressable.

A sample may be attached to the sample carrier by a cleavable photoreactive moiety. Upon irradiating the cleavable photoreactive moiety with radiation (e.g. from a laser in the laser system of the laser ablation sampling system), the photoreactive moiety can cleave to release sample material. The sample carrier may comprise (i) a cleavable photoreactive moiety that couples the sample to the sample carrier and (ii) a desorption film as discussed above. In this situation, a first laser radiation pulse may be used to cause cleavage of the photoreactive moiety and a second laser radiation pulse may be used to target the desorption film to cause separation of the sample from the sample carrier by lifting (or a thermal energy pulse introduced by other means may be used to heat the desorption film and so cause separation of sample material from the sample carrier). The first and second pulses may be of different wavelengths. Thus in some methods (e.g. comprising both ablation and desorption), separation of the sample from the sample carrier may involve multiple laser pulses of different wavelengths. In some instances, cleavage of the photoreactive moiety and lifting may be accomplished by the same laser pulse.

The sample carrier may include a coating or layer of a chemically reactive species that imparts kinetic energy to the sample to release the sample from the surface. For example, a chemically reactive species may release a gas such as, for example, H₂, CO₂, N₂ or hydrochlorofluorocarbons. Examples of such compounds include blowing and foaming agents, which release gas upon heating. Generation of gas can be used to impart kinetic energy to desorbing sample material that can improve the reproducibility and direction of release of the material.

A sample carrier may comprise photoinitiated chemical reactants that undergo an exothermic reaction to generate heat for desorbing sample material. The coating of the carrier, or indeed particular chemical linkages in that carrier, discussed in the above paragraphs (that is irradiated by the laser to release the slug of sample material from the carrier) is an example of a material that can be targeted by a wavelength of laser radiation.

The sites on the sample carrier from which slugs of sample material are to be desorbed may be mounted and/or coupled to MEMS devices configured to facilitate release of a biological material from the discrete sites on a carrier.

A slug of the sample can be released or desorbed from a discrete site using nano-heaters, bubble jets, piezoelectrics, ultrasonics, electrostatics, or a combination of any of the foregoing.

Each, or a combination, of these techniques permits ordered detachment of a slug of sample material from the sample carrier. However, often, the location on the sample that is of interest does not represent a discrete entity, such as a lone cell, at a discrete site which can be easily desorbed in isolation. Instead, the cell of interest may be surrounded by other cells or material, of which analysis is not required or desired. Trying to perform desorption (e.g. lifting) of the feature/region of interest may therefore desorb both the cell of interest and surrounding material together. Atoms, such as labelling atoms which are used in elemental tags (see discussion below), from the surrounding area of the sample (e.g. from other cells which have been labelled) that are carried in a desorbed slug of material in addition to the specific feature/region (e.g. cell) of interest could therefore contaminate the reading for the location of interest.

The techniques of ablation and desorption (such as by lifting) can be combined in a single method. For example, to perform precise desorption of a feature/region (e.g. cell) of interest on a biological sample, e.g. a tissue section sample or cell suspension dispersion, on the sample carrier, laser ablation can be used to ablate the area around the cell of interest to clear it of other material. After clearing the surrounding area by ablation, the feature/region of interest can then be desorbed from the sample carrier, and then ionised and analyzed by mass spectrometry in line with standard mass cytometry or mass spectrometry procedures. In line with the above discussion, thermal, photolytic, chemical, or physical techniques can be used to desorb material from a feature/region of interest, optionally after ablation has been used to clear the area surrounding the location that will be desorbed. Often, lifting will be employed, to separate the slug of material from the sample carrier (e.g. a sample carrier which has been coated with a desorption film to assist the lifting procedure, as discussed above with regard to desorption of discrete slugs of sample material).

The feature/region of interest can be identified by another technique before the laser ablation and desorption (e.g. by lifting) is performed. The inclusion of a camera (such as a charged coupled device image sensor based (CCD) camera or a CMOS camera or an active pixel sensor based camera), or any other light detecting means as described in the preceding sections is one way of enabling these techniques, for both online and offline analyses. The camera can be used to scan the sample to identify cells of particular interest or features/regions of particular interest (for example cells of a particular morphology). Once such locations have been identified, the locations can be lifted after laser pulses have been directed at the area around the feature/region of interest to clear other material by ablation before the location (e.g. cell) is lifted. This process may be automated (where the system both identifies, ablates and lifts the feature(s)/region(s) of interest) or semi-automated process (where the user of the system, for example a clinical pathologist, identifies the feature(s)/region(s) of interest, following which the system then performs ablation and lifting in an automated fashion). This enables a significant increase in the speed at which analyses can be conducted, because instead of needing to ablate the entire sample to analyze particular cells, the cells of interest can be specifically ablated.

The camera can record the image from a microscope (e.g. a confocal microscope). The identification may be by light microscopy, for example by examining cell morphology or cell size, or on whether the cell is a discrete single cell (in contrast to a member of a clump of cells). Sometimes, the sample can be specifically labelled to identify the feature(s) (e.g. cell(s)) of interest. Often, fluorescent markers are used to specifically stain the cells of interest (such as by using labelled antibodies or labelled nucleic acids), as discussed above in relation to methods of ablating visually-identified features/regions of interest; that section is not repeated here in full in the interests of brevity, but one of skill in the art will immediately appreciate that the features of those methods can be applied to desorption based methods and that this is within the technical teaching of this document. A high resolution optical image is advantageous in this coupling of optical techniques and lifting, because the accuracy of the optical image then determines the precision with which the ablating laser source can be directed to ablate the area surrounding the cell of interest which can subsequently be ablated.

Sometimes, no data are recorded from the ablation performed to clear the area around the location to be desorbed (e.g. the cell of interest). Sometimes, data is recorded from the ablation of the surrounding area. Useful information that can be obtained from the surrounding area includes what target molecules, such as proteins and RNA transcripts, are present in the surrounding cells and intercellular milieu. This may be of particular interest when imaging solid tissue samples, where direct cell-cell interactions are common, and what proteins etc. are expressed in the surrounding cells may be informative on the state of the cell of interest.

### Camera

The camera used in the desorption based sampling system can be as described above for the laser ablation based sampling system, and the discussion for the camera of the laser ablation based sampling system should be read in here.

### Sample Chamber

The sample chamber used in the desorption based sampling system can be as described above for the laser ablation based sampling system. In instances where sampling of large slugs of sample material is being undertaken, the skilled practitioner will appreciate that gas flow volumes may need to be increased to ensure that the slug of material is entrained in the flow of gas and carried into the transfer conduit for transport to the ionisation system.

### Transfer Conduit

The sample chamber used in the desorption based sampling system can be as described above for the laser ablation based sampling system. In instances where sampling of large slugs of sample material is being undertaken, the skilled practitioner will appreciate that the diameter of the lumen of the conduit will need to be appropriately sized to accommodate any slugs without the slug contacting the side of the lumen (because any contact may lead to fragmentation of the slug, and to the overlapping of signals—where atoms from the slug resulting the nth desorption event are spread into the detection window for the n+1th or subsequent slugs).

### Ionisation System of the Desorption Based System

In many instances, the lifting techniques discussed above involve the removal of relatively large slugs of sample material (10 nm to 10 μm, from 100 nm to 10 μm, and in certain instances from 1 μm to 100 μm) which have not been converted into particulate and vaporous material. Accordingly, an ionisation technique which is capable of vaporising and atomising this relatively large quantity of material is required.

### Inductively Coupled Plasma Torch

One such suitable ionisation system is an inductively coupled plasma, as already discussed above in the section beginning on page 95 in relation to laser ablation based sampling and ionisation systems.

### Optional Further Components of the Desorption Based Sampling and Ionisation System

### Ion Deflector

The ion deflector used in the desorption based sampling system can be as described above for the laser ablation based sampling system. Given the potential for desorption based sampling to remove intact large slugs of sample material, ion deflectors can be particularly useful in this kind of system for protecting the detector.

c. Laser Desorption/Ionisation Systems

A laser desorption/ionisation based analyser typically comprises two components. The first is a system for the generation of ions from the sample for analysis. In this apparatus, this is achieved by directing a laser beam onto the sample to generate ions; herein it is called a laser desorption ion generation system. These ejected sample ions (including any detectable ions from labelling atoms as discussed below) can be detected by a detector system (the second component) for instance a mass spectrometer (detectors are discussed in more detail below). This technique is known as laser desorption/ionisation mass spectrometry (LDI-MS). LDI is different from the desorption based sampling systems discussed in more detail below, because in the desorption based sampling system the sample material is desorbed as charge neutral slugs of material which are subsequently ionised to form elemental ions. On the contrary, here, ions are produced directly as a result of irradiation of the sample by the laser and no separate ionisation system is required.

The laser desorption ion generation system comprises: a laser; a sample chamber for housing the sample onto which radiation from the laser is directed; and ion optics that take ions generated from the sample and direct them to the detector for analysis. Accordingly, the invention provides an apparatus for analysing a sample comprising: a. a sample chamber to house the sample; b. a laser, adapted to desorb and ionize material from the sample, forming ions; c. ion optics, arranged to sample the ions formed by desorption ionisation, and to direct them away from sample towards the detector; and d. a detector to receive ions from said ion optics and to analyse said ions. In some embodiments, the apparatus comprises a laser adapted to desorb and ionize material from the sample, forming elemental ions, and wherein the detector receives the elemental ions from said sampling and ionisation system and analyses said elemental ions.

In this process some molecules reach an energy level at which they desorb from the sample and become ionised. The ions may arise as primary ions directly as a result of the laser irradiation or as secondary ions, formed by collision of charge neutral species with the primary ions (e.g. proton transfer, cationization and electron capture). In some instances, ionisation is assisted by compounds (e.g. a matrix) added to the sample as the sample is being prepared, as discussed below.

It will be apparent to those skilled in the art that laser desorption ionisation may be used to sample the fused reference particles, in addition to sampling mass tags when imaging a sample. However, it will also be apparent that the laser intensity will need to be varied depending on the material being sampled. Thus, as the fused reference particles are significantly thicker than the layer of sample material being sampled, a much higher laser intensity during laser desorption ionisation may be required to desorb and ionise all the material of a reference particle in order to be able to obtain an integral signal intensity corresponding to all the material in the whole fused particle. Alternatively, multiple lower energy laser shots can be fired at the same spot in succession to ablate through the fused particle.

### 2. Mass Detector System

Exemplary types of mass detector system include quadrupole, time of flight (TOF), magnetic sector, high resolution, single or multicollector based mass spectrometers.

The time taken to analyse the ionised material will depend on the type of mass analyser which is used for detection of ions. For example, instruments which use Faraday cups are generally too slow for analysing rapid signals. Overall, the desired imaging speed, resolution and degree of multiplexing will dictate the type(s) of mass analyser which should be used (or, conversely, the choice of mass analyser will determine the speed, resolution and multiplexing which can be achieved).

Mass spectrometry instruments that detect ions at only one mass-to-charge ratio (m/Q, commonly referred to as m/z in MS) at a time, for example using a point ion detector, will give poor results in imaging detecting. Firstly, the time taken to switch between mass-to-charge ratios limits the speed at which multiple signals can be determined, and secondly, if ions are at low abundance then signals can be missed when the instrument is focused on other mass-to-charge ratios. Thus it is preferred to use a technique which offers substantially simultaneous detection of ions having different m/Q values.

### Detector Types

### Quadrupole Detector

Quadrupole mass analysers comprise four parallel rods with a detector at one end. An alternating RF potential and fixed DC offset potential is applied between one pair of rods and the other so that one pair of rods (each of the rods opposite each other) has an opposite alternative potential to the other pair of rods. The ionised sample is passed through the middle of the rods, in a direction parallel to the rods and towards the detector. The applied potentials affect the trajectory of the ions such that only ions of a certain mass-charge ratio will have a stable trajectory and so reach the detector. Ions of other mass-charge ratios will collide with the rods.

### Magnetic Sector Detector

In magnetic sector mass spectrometry, the ionised sample is passed through a curved flight tube towards an ion detector. A magnetic field applied across the flight tube causes the ions to deflect from their path. The amount of deflection of each ion is based on the mass to charge ratio of each ion and so only some of the ions will collide with the detector—the other ions will be deflected away from the detector. In multicollector sector field instruments, an array of detectors is be used to detect ions of different masses. In some instruments, such as the ThermoScientific Neptune Plus, and Nu Plasma II, the magnetic sector is combined with an electrostatic sector to provide a double-focussing magnetic sector instrument that analyses ions by kinetic energy, in addition to mass to charge ratio. In particular those multidetectors having a Mattauch-Herzog geometry can be used (e.g. the SPECTRO MS, which can simultaneously record all elements from lithium to uranium in a single measurement using a semiconductor direct charge detector). These instruments can measure multiple m/Q signals substantially simultaneously. Their sensitivity can be increased by including electron multipliers in the detectors. Array sector instruments are always applicable, however, because, although they are useful for detecting increasing signals, they are less useful when signal levels are decreasing, and so they are not well suited in situations where labels are present at particularly highly variable concentrations.

### Time of Flight (TOF) Detector

A time of flight mass spectrometer comprises a sample inlet, an acceleration chamber with a strong electric field applied across it, and an ion detector. A packet of ionised sample molecules is introduced through the sample inlet and into the acceleration chamber. Initially, each of the ionised sample molecules has the same kinetic energy but as the ionised sample molecules are accelerated through the acceleration chamber, they are separated by their masses, with the lighter ionised sample molecules travelling faster than heaver ions. The detector then detects all the ions as they arrive. The time taking for each particle to reach the detector depends on the mass to charge ratio of the particle.

Thus a TOF detector can quasi-simultaneously register multiple masses in a single sample. In theory TOF techniques are not ideally suited to ICP ion sources because of their space charge characteristics, but TOF instruments can in fact analyse an ICP ion aerosol rapidly enough and sensitively enough to permit feasible single-cell imaging. Whereas TOF mass analyzers are normally unpopular for atomic analysis because of the compromises required to deal with the effects of space charge in the TOF accelerator and flight tube, tissue imaging according to the subject disclosure can be effective by detecting only the labelling atoms, and so other atoms (e.g. those having an atomic mass below 100) can be removed. This results in a less dense ion beam, enriched in the masses in (for example) the 100-250 dalton region, which can be manipulated and focused more efficiently, thereby facilitating TOF detection and taking advantage of the high spectral scan rate of TOF. Thus rapid imaging can be achieved by combining TOF detection with choosing labelling atoms that are uncommon in the sample and ideally having masses above the masses seen in an unlabelled sample e.g. by using the higher mass transition elements. Using a narrower window of label masses thus means that TOF detection to be used for efficient imaging.

Suitable TOF instruments are available from Tofwerk, GBC Scientific Equipment (e.g. the Optimass 9500 ICP-TOFMS), and Fluidigm Canada (e.g. the CyTOF™ and CyTOF™2 instruments). These CyTOF™ instruments have greater sensitivity than the Tofwerk and GBC instruments and are known for use in mass cytometry because they can rapidly and sensitively detect ions in the mass range of rare earth metals (particularly in the m/Q range of 100-200; see Bandura et al. (2009; Anal. Chem., 81:6813-22)). Thus these are preferred instruments for use with the disclosure, and they can be used for imaging with the instrument settings already known in the art e.g. Bendall et al. (2011; Science 332, 687-696) & Bodenmiller et al. (2012; Nat. Biotechnol. 30:858-867). Their mass analysers can detect a large number of markers quasi-simultaneously at a high mass-spectrum acquisition frequency on the timescale of high-frequency laser ablation or sample desorption. They can measure the abundance of labelling atoms with a detection limit of about 100 per cell, permitting sensitive construction of an image of the tissue sample. Because of these features, mass cytometry can now be used to meet the sensitivity and multiplexing needs for tissue imaging at subcellular resolution. By combining the mass cytometry instrument with a high-resolution laser ablation sampling system and a rapid-transit low-dispersion sample chamber it has been possible to permit construction of an image of the tissue sample with high multiplexing on a practical timescale.

The TOF may be coupled with a mass-assignment corrector. The vast majority of ionisation events generate M⁺ ions, where a single electron has been knocked out of the atom. Because of the mode of operation of the TOF MS there is sometimes some bleeding (or cross-talk) of the ions of one mass (M) into the channels for neighbouring masses (M±1), in particular where a large number of ions of mass M are entering the detector (i.e. ion counts which are high, but not so high that an ion deflector positioned between the sampling ionisation system and MS would prevent them from entering the MS, if the apparatus were to comprise such an ion deflector). As the arrival time of each M⁺ ion at the detector follows a probability distribution about a mean (which is known for each M), when the number of ions at mass M⁺ is high, then some will arrive at times that would normally be associated with the M−1⁺ or M+1⁺ ions. However, as each ion has a known distribution curve upon entering the TOF MS, based on the peak in the mass M channel it is possible to determine, the overlap of ions of mass M into the M±1 channels (by comparison to the known peak shape). The calculation is particularly applicable for TOF MS, because the peak of ions detected in a TOF MS is asymmetrical. Accordingly it is therefore possible to correct the readings for the M−1, M and M+1 channels to appropriately assign all of the detected ions to the M channel. Such corrections have particular use in correcting imaging data due to the nature of the large packets of ions produced by sampling and ionisation systems such as those disclosed herein involving laser ablation (or desorption as discussed below) as the techniques for removing material from the sample. Programs and methods for improving the quality of data by de-convoluting the data from TOF MS are discussed in WO2011/098834, U.S. Pat. No. 8,723,108 and WO2014/091243.

### Dead-Time Corrector

As noted above, signals in the MS are detected on the basis of collisions between ions and the detector, and the release of electrons from the surface of the detector hit by the ions. When a high count of ions is detected by the MS resulting in the release of a large number of electrons, the detector of the MS can become temporarily fatigued, with the result that the analog signal output from the detector is temporarily depressed for one or more of the subsequent packets of ions. In other words, a particularly high count of ions in a packet of ionised sample material causes a lot of electrons to be released from the detector surface and secondary multiplier in the process of detecting the ions from that packet of ionised sample material, meaning that fewer electrons are available to be released when the ions in subsequent packets of ionised sample material hit the detector, until the electrons in the detector surface and secondary amplifier are replenished.

Based on a characterisation of the behaviour of the detector, it is possible to compensate for this dead-time phenomenon. A first step is to analyse the ion peak in the analog signal resulting from the detection of the nth packet of ionised sample material by the detector. The magnitude of the peak may be determined by the height of the peak, by the area of the peak, or by a combination of peak height and peak area.

The magnitude of the peak is then compared to see if it exceeds a predetermined threshold. If the magnitude is below this threshold, then no correction is necessary. If the magnitude is above the threshold, then correction of the digital signal from at least one subsequent packet of ionised sample material will be performed (at least the (n+1)th packet of ionised sample material, but possibly further packets of ionised sample material, such as (n+2)th, (n+3)th, (n+4)th etc.) to compensate for the temporary depression of the analog signal from these packets of ionised sample material resulting from the fatiguing of the detector caused by the nth packet of ionised sample material. The greater the magnitude of the peak of the nth packet of ionised sample material, the more peaks from subsequent packets of ionised sample material will need to be corrected and the magnitude of correction will need to be greater. Methods for correcting such phenomena are discussed in Stephan et al. (1994; Vac. Sci. Technol. 12:405), Tyler and Peterson (2013; Surf Interface Anal. 45:475-478), Tyler (2014; Surf Interface Anal. 46:581-590), WO2006/090138 and U.S. Pat. No. 6,229,142, and these methods can be applied by the dead-time corrector to the data, as described herein.

### Mass Imaging Based on Optical Emission Spectra Detection

1. Sampling and Ionisation Systems

a. Laser Ablation Based Sampling and Ionising System

The laser ablation sampling system sampling system described above in relation to mass-based analysers can be employed in an OES detector-based system. For detection of atomic emission spectra, most preferably, an ICP is used to ionise the sample material removed from the sample, but any hard ionisation technique that can produce elemental ions can be used.

As appreciated by one of skill in the art, certain optional further components of the laser ablation based sampling and ionising system above, described in relation to avoiding overload of the mass-based detector, may not be applicable to all OES detector-based systems, and would not be incorporated, if inappropriate, by the skilled artisan. Furthermore, the skilled artisan will appreciate that while OES can detect elements, it cannot distinguish between isotopes of the element. Accordingly, where target SBPs/analytes are to be distinctively analysed, OES should be conducted with reagents labelled with different elements, rather isotopes of the same element.

b. Desorption Based Sampling and Ionising System

The desorption-based sampling system described above in relation to mass-based analysers can be employed in an OES detector-based system. For detection of atomic emission spectra, most preferably, an ICP is used to ionise the sample material removed from the sample, but any hard ionisation technique that can produce elemental ions can be used.

As appreciated by one of skill in the art, certain optional further components of the desorption based sampling and ionising system above, described in relation to avoiding overload of the mass-based detector, may not be applicable to all OES detector-based systems, and would not be incorporated, if inappropriate, by the skilled artisan.

2. Photodetectors

Exemplary types of photodetectors include photomultipliers and charged-coupled devices (CCDs). Photodetetors may be used to image the sample and/or identify a region of interest prior to imaging by elemental mass spectrometry.

Photomultipliers comprise a vacuum chamber comprising a photocathode, several dynodes, and an anode. A photon incident on the photocathode causes the photocathode to emit an electron as a consequence of the photoelectric effect. The electron is multiplied by the dynodes due to the process of secondary emission to produce a multiplied electron current, and then the multiplied electron current is detected by the anode to provide a measure of detection of electromagnetic radiation incident on the photocathode. Photomultipliers are available from, for example, ThorLabs.

A CCD comprises a silicon chip containing an array of light-sensitive pixels. During exposure to light, each pixel generates an electric charge in proportion to the intensity of light incident on the pixel.

After the exposure, a control circuit causes a sequence of transfers of electric charge to produce a sequence of voltages. These voltages can then be analysed to produce an image. Suitable CCDs are available from, for example, Cell Biosciences.

### Constructing an Image

The apparatus above can provide signals for multiple atoms in packets of ionised sample material removed from the sample (be that by ablation, ion bombardment or any other technique). Detection of an atom in a packet of sample material reveals its presence at the position of ablation, be that because the atom is naturally present in the sample or because the atom has been localised to that location by a labelling reagent. By generating a series of packets of ionised sample material from known spatial locations on the sample's surface the detector signals reveal the location of the atoms on the sample, and so the signals can be used to construct an image of the sample. By labelling multiple targets with distinguishable labels it is possible to associate the location of labelling atoms with the location of cognate targets, so the method can build complex images, reaching levels of multiplexing which far exceed those achievable using existing techniques. The images generated by the methods can reproduce the staining patterns and the proportion of cells expressing a given marker as determined by IFM, thereby confirming the method's suitability for imaging.

Assembly of signals into an image will use a computer and can be achieved using known techniques and software packages. For instance, the GRAPHIS package from Kylebank Software may be used, or other packages such as TERAPLOT can also be used. Imaging using MS data from techniques such as MALDI-MSI is known in the art e.g. Robichaud et al. (2013; J Am Soc Mass Spectrom 24 5:718-21) discloses the ‘MSiReader’ interface to view and analyze MS imaging files on a Matlab platform, and Klinkert et al. (2014; Int J Mass Spectrom http://dx.doi.org/10.1016/j.ijms.2013.12.012) discloses two software instruments for rapid data exploration and visualization of both 2D and 3D MSI data sets in full spatial and spectral resolution e.g. the ‘Datacube Explorer’ program.

Images obtained using the methods disclosed herein can be further analysed e.g. in the same way that IHC results are analysed. For instance, the images can be used for delineating cell sub-populations within a sample, and can provide information useful for clinical diagnosis. Similarly, SPADE analysis can be used to extract a cellular hierarchy from the high-dimensional cytometry data which methods of the disclosure provide (Qiu et al. (2011; Nat. Biotechnol. 29:886-91)).

### Computer Control of Methods Disclosed Herein

The methods disclosed herein may also be provided as a computer program product including a non-transitory, machine-readable medium having stored thereon instructions that may be used to program a computer (or other electronic device) to perform the processes described herein. The machine-readable medium may include, but is not limited to, hard drives, floppy diskettes, optical disks, CD-ROMs, DVD-ROMs, ROMs, RAMs, EPROMs, EEPROMs, magnetic or optical cards, solid-state memory devices, or other types of media/computer-readable medium suitable for storing electronic instructions. Accordingly, the invention also provides a machine-readable medium comprising instructions for performing a method as disclosed herein.

### Definitions

The term “comprising” encompasses “including” as well as “consisting” e.g. a composition “comprising” X may consist exclusively of X or may include something additional e.g. X+Y.

The term “about” in relation to a numerical value x is optional and means, for example, x+10%.

The word “substantially” does not exclude “completely” e.g. a composition which is “substantially free” from Y may be completely free from Y. Where necessary, the word “substantially” may be omitted from the definition of the disclosure.

Molecular weights (Mₙ) and polymer dispersity indexes (PDI=M_{w}/Mₙ) were obtained by aqueous gel permeation chromatography (GPC), performed at room temperature, with 0.2 M NaNO₃ as the eluent. Molecular weights are referenced to polyethylene glycol (PEG) standards.

### EXAMPLES

### Example 1—Immobilising and Sampling Fused Reference Particles Prior to Imaging of a Sample

An imaging sample carrier of the invention was prepared according to the following method. Firstly, a sample of spleen was prepared on the sample carrier using methods known in the art. The spleen sample was labelled with SBP-mass tags (the targets of the SBPs were alphaSMA, VIM, CD11b, CD45, CD4, CD68, CD20, CD8a, Collagen1, CD3, HistoneH3, DNA1, DNA3—available from Fluidigm Canada). The sample was then inserted into an HTI45 imaging mass cytometer and an area of 500×500 μm was ablated. The sample carrier was removed from the imaging mass cytometer. In a separate 1.5 mL centrifuge tube, 2 μL of a concentrated suspension of EQ4 beads (1.3×10¹⁰ particles per ml PN: F00306 Lot: X13H2302) was diluted in 300 μL DIW and vortexed. Then, 2 μL of the diluted EQ4 bead solution was pipetted onto an area of the slide separate from spleen sample. The pipette tip was used to smear the sample to facilitate evaporation of the aqueous solvent. The slide was then inspected under a microscope to confirm that the beads were isolated individually on the slide. The sample carrier was then placed on a hotplate set at 200° C. with a crystallization dish placed over top of slide. The slide was then heated for ten minutes at 200° C. The sample carrier was then removed from the hotplate and allowed to cool. The reference particles were then inspected again using a optical microscope to confirm that the beads had fused to the slide (indicated through an increase in the diameter of the reference particle).

### Example 2—Effect of the Fusing the Reference Particle on the Sample Carrier on Biological Samples

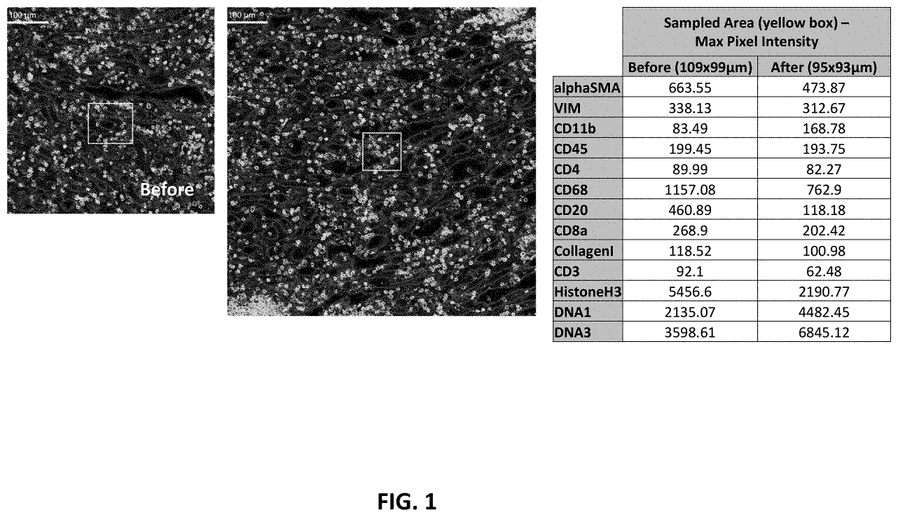

Experiments were conducted to assess whether the process of fusing reference particles onto the sample carrier would affect a biological sample prepared onto a sample carrier slide. Thus, an imaging mass calibrator comprising a sample was prepared according to the method detailed in example 1. The sample was then loaded into an imaging mass cytometer and imaged. The image of the sample was acquired, and a maximum signal intensity detected for each of the labelling atoms in the mass tags labelling the sample. The sample carrier was removed from the instrument and a portion of the sample carrier not comprising the sample was contacted with a suspension of reference particles, following the method disclosed in Example 1. The reference particles were then fused to the sample carrier also using the method disclosed in example 1. Following fusion of the reference particles to the sample carrier, the spleen sample was reintroduced into the imaging mass cytometer and imaged again. As shown in FIG. 1, the max signal intensity detected from each of the mass tags was observed to remain substantially constant following heating of the sample carrier. This indicates that the method of fusing the reference particles to the sample carrier does not significantly affect the sample, such that the imaging mass calibrators made according to the method of the invention can provide accurate images of samples isolated from tissues, and provide absolute quantitation of the copy number of the analytes present in the sample.

### Example 3—Assessment of Imaging Mass Cytometer Stability During Imaging Using an Imaging Mass Cytometer Calibrator

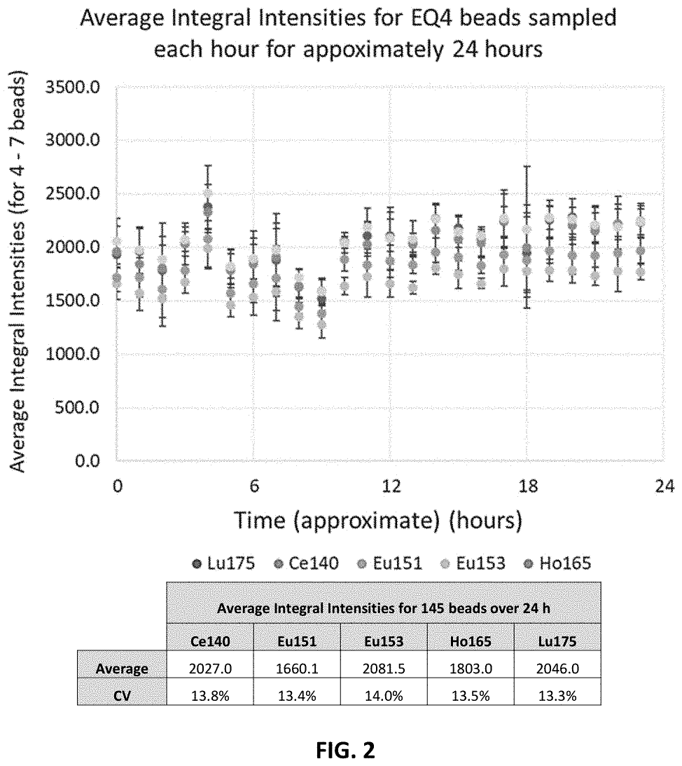

The stability of an imaging mass cytometer was assessed using the imaging mass calibrator of the invention. After preparing an imaging mass calibrator in accordance with the method detailed in example 1, the sample carrier was inserted into an imaging mass cytometer and six complete beads were ablated and an average integral signal intensity per fused bead calculated. A 750 μm×750 μm area of the sample was then imaged requiring approximately one hour of imaging time. After the area had been imaged, another six beads were ablated and an average integral intensity per fused bead calculated at this time point. This process was then continued, with further 750 μm×750 μm areas of sample being imaged, followed by the ablation of another six beads. Thus, six beads were ablated between each area of the sample imaged, until 23 tissue areas and 24 sets of six beads had been ablated, requiring approximately 24 hours in imaging time in total. As shown in FIG. 2, no significant variation in the average integral signal intensities was observed for each of the labelling atoms detected in the mass channels of the detector during the imaging of the sample. The results demonstrate how the imaging mass calibrator of the invention can be used to monitor the performance of an imaging mass cytometer, in this instance observing no variation in instrument sensitivity over 24 hours. Furthermore, the standard deviation of the average integral signal intensities detected for each set of beads sampled over the 24 hour period were less than 15%, further demonstrating good instrument stability during the run. The average integral signal intensities and their standard deviations are detected during the course of the imaging run are given in Table 1.

TABLE 1
Average integral signal intensities perfused reference particle (6 beads) and the
standard deviation detected for each set of beads sampled during the imaging run.

| | Ce140 Integral | | Eu151 Integral | | Eu153 Integral | | Ho165 Integral | | Lu175 Integral | |
|---|---|---|---|---|---|---|---|---|---|---|
| | Intensities | | Intensities | | Intensities | | Intensities | | Intensities | |
| Time (h) | Average | CV | Average | CV | Average | CV | Average | CV | Average | CV |
| 0 | 1961.3 | 12.1% | 1659.5 | 9.0% | 2058.9 | 10.3% | 1718.6 | 7.5% | 1930.7 | 6.1% |
| 1 | 1843.9 | 6.6% | 1573.0 | 10.6% | 1973.6 | 11.0% | 1717.6 | 10.7% | 1961.6 | 10.9% |
| 2 | 1806.3 | 16.4% | 1518.2 | 16.7% | 1885.5 | 18.2% | 1606.4 | 16.5% | 1785.2 | 13.3% |
| 3 | 2032.1 | 5.7% | 1678.3 | 6.4% | 2074.9 | 5.6% | 1787.4 | 7.3% | 2035.4 | 9.6% |
| 4 | 2333.6 | 8.2% | 1994.3 | 9.1% | 2507.0 | 10.3% | 2077.5 | 13.2% | 2378.3 | 8.9% |
| 5 | 1782.2 | 8.8% | 1460.2 | 7.3% | 1819.6 | 9.0% | 1574.3 | 8.9% | 1817.4 | 8.8% |
| 6 | 1841.1 | 10.1% | 1533.6 | 10.9% | 1895.9 | 13.5% | 1659.1 | 10.7% | 1879.5 | 11.1% |
| 7 | 1908.3 | 14.2% | 1586.0 | 17.4% | 1973.7 | 17.5% | 1713.8 | 17.5% | 1883.6 | 18.2% |
| 8 | 1639.5 | 9.4% | 1349.2 | 7.9% | 1716.1 | 4.7% | 1448.7 | 4.8% | 1629.0 | 5.9% |
| 9 | 1591.3 | 7.7% | 1280.3 | 10.0% | 1588.9 | 6.8% | 1382.8 | 6.5% | 1519.7 | 4.5% |
| 10 | 2042.6 | 4.8% | 1635.7 | 4.9% | 2045.1 | 2.7% | 1885.6 | 5.5% | 2056.8 | 2.2% |
| 11 | 2027.3 | 9.8% | 1725.9 | 10.9% | 2191.4 | 8.0% | 1840.0 | 5.9% | 2112.6 | 6.1% |
| 12 | 2085.1 | 14.0% | 1657.4 | 7.6% | 2088.1 | 11.7% | 1871.5 | 8.1% | 2112.5 | 7.1% |
| 13 | 2030.7 | 4.8% | 1623.4 | 3.4% | 2080.2 | 8.0% | 1835.8 | 4.4% | 2043.1 | 4.4% |
| 14 | 2164.2 | 3.7% | 1804.9 | 3.1% | 2264.0 | 5.8% | 1955.3 | 6.6% | 2272.4 | 6.0% |
| 15 | 2082.5 | 10.0% | 1750.8 | 7.7% | 2147.1 | 7.0% | 1912.3 | 6.8% | 2179.4 | 4.9% |
| 16 | 2043.1 | 6.3% | 1663.5 | 3.0% | 2111.3 | 3.7% | 1828.2 | 4.1% | 2078.4 | 3.2% |
| 17 | 2245.9 | 11.5% | 1797.9 | 8.9% | 2269.0 | 11.6% | 1931.8 | 7.9% | 2240.5 | 6.3% |
| 18 | 1998.7 | 18.6% | 1779.5 | 13.6% | 2170.2 | 27.2% | 1878.8 | 23.8% | 1945.1 | 16.6% |
| 19 | 2277.7 | 4.9% | 1787.7 | 5.8% | 2276.3 | 4.7% | 1972.4 | 6.1% | 2258.8 | 7.9% |
| 20 | 2203.3 | 7.8% | 1783.9 | 6.7% | 2263.8 | 8.6% | 1922.7 | 9.0% | 2287.1 | 7.3% |
| 21 | 2157.2 | 10.7% | 1731.2 | 5.0% | 2214.0 | 7.3% | 1922.5 | 5.7% | 2187.4 | 6.1% |
| 22 | 2222.9 | 11.6% | 1776.5 | 10.5% | 2193.0 | 9.5% | 1945.2 | 8.0% | 2216.6 | 6.6% |
| 23 | 2251.1 | 7.0% | 1770.7 | 4.1% | 2244.1 | 6.3% | 1970.7 | 6.0% | 2236.5 | 5.4% |

### Example 4—Controllably Dispersing EQ Beads onto a Sample Carrier

Investigations were conducting into potential solvents and conditions suitable for contacting metal doped beads to a sample carrier to ensure that the majority beads of the beads are localised discretely on the sample. Thus, two dilutions of a concentrated EQ4 solution (PN: F00306 Lot: X13H2302) were made in two different solvents. Thus, 2 μL of the concentrated EQ4 bead solution was added to 200 μL deionised water (DIW): Reference for suspension=DIW-1. In addition, 2 μL of the concentrated EQ4 bead solution was added to 200 μL ethanol (EtOH): Reference for suspension=EtOH-1. Further, 100 μL of the DIW-1 suspension was added to 100 μL of DIW. Reference for solution=DIW-2. Finally, 100 μL of the EtOH-1 suspension was added to 100 μL of EtOH. Reference for solution=EtOH-2.

Then, 2 μL of each solution was pipetted onto a sample carrier (a glass slide).

For the DIW suspensions, a rectangular area (˜8 mm×˜14 mm) was marked out on the back of the sample carrier before 2 μL of either the DIW-1 or DIW-2 suspension was pipetted into the middle of rectangular area. The pipette tip was then used to smear the water droplet around the area of the rectangle to facilitate evaporation.

For the EtOH suspensions, a rectangular area (˜8 mm×˜14 mm) was marked out on the back of the sample carrier before 2 μL of either the EtOH-1 or EtOH-2 suspension was initially pipetted near to the bottom of the rectangle and the suspension slowly dispensed while moving the pipette tip to the top of the rectangle. The EtOH quickly evaporated.

Each slide prepared according to these methods was then inspected under a microscope to confirm that the sample has dried and that the beads were spread sufficiently uniformly across the slide, with the majority of beads located in discrete locations such that beads are separated from one another.

Both dilutions of the beads in both the solvents tested were found to provide a sufficiently uniform distribution of beads on the slides with a majority of the beads isolated as individual beads. Following fusing of these beads onto the slides, those located in isolation can therefore easily be ablated, allowing for an accurate quantitation of the average integral signal intensity per fused reference particle.

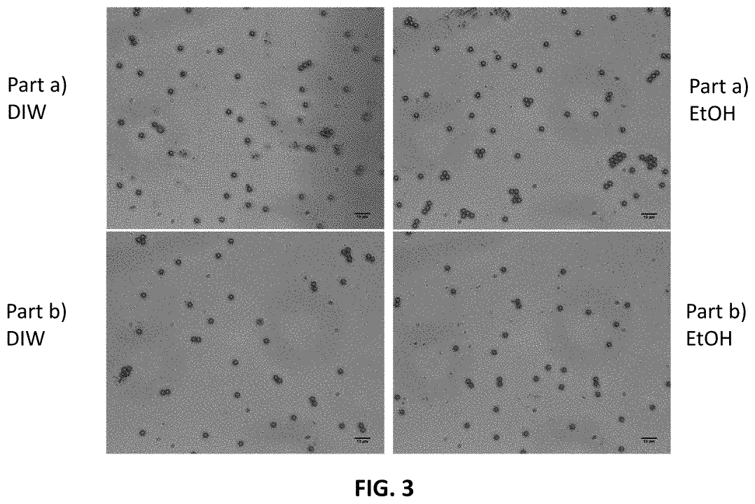

FIG. 3 is an optical microscope image of beads positioned on a sample carrier using the above method. The images were taken before the beads were fused to the sample carrier. An average bead diameter of 3.2 μm (volume=17.2 μm³) was calculated from 38 beads measured using the ImageJ Detect Circles plugin.

### Example 5—Conditions for Fusing EQ4 Reference Particles to a Sample Carrier

Imaging mass calibrators comprising a sample carrier were prepared according to the method recited in example 4, using the EtOH-2 suspension of beads. The sample carriers were then placed onto a hotplate set at a range of temperatures for 10-30 minutes. After heating, the sample carrier was removed from the hot plate and allowed to cool. The sample carriers were then inspected under an optical microscope to determine whether the reference particles were fused to the sample carrier. The results of this study into the effects of heating sample carriers comprising EQ4 beads at different temperatures are provided in Table 2.

TABLE 2
Effect of temperature on EQ4 reference
particles on a sample carrier

| Hotplate set | Slide Probe | Sufficient to fuse |
|---|---|---|
| Temp. (° C.) | Temp. (° C.) | reference particles: |
| 145 | ~126 | No |
| 175 | ~153 | No |
| 200 | ~175 | Yes |

### Example 6—Investigation into the Effect of Duration of Heating

Imaging mass calibrators comprising a sample carrier were prepared according to the method recited in example 4. The sample carriers were placed onto a hotplate set at 200 C for either 10, 20, or 30 minutes. After heading, the sample carriers were removed from the hotplate and allowed to cool.

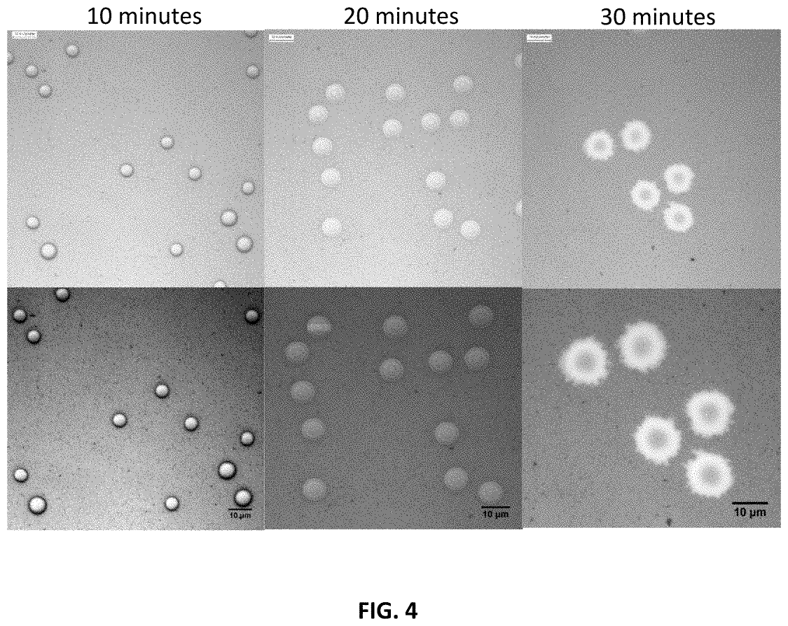

FIG. 4 depicts SEM/TEM images of the beads on the samples carriers prepared according to the method recited in Example 3, that have been heated for either 10, 20, or 30 minutes at 175° C. (hot plate set at 200° C.). The sample carriers were then inspected under an optical microscope to determine whether the reference particles were fused to the sample carrier. A reference particle was judged to be fused to the sample carrier when an increase in reference particle diameter was observed using the ImageJ Detect Circles plugin. The average diameter of the reference particles was calculated for each sample carrier. The reference particle diameter of the bead was found to increase from an average of 3.2 μm (volume=17.2 μm³) before heating, to a 5.3 μm average after heating for 10 minutes, an 8.4 μm average after heating for 20 minutes, and an 12.4 μm average after heating for 30 minutes at 175° C.

Furthermore, the average integral signal intensity of the beads was determined for the sample carriers following heating. The average integral signal intensity was found to decrease with increasing heating time. The results are provided in Table 3.

TABLE 3
effect of heating time on the average maximum and
integral signal intensities detected for the

[symbol]

| | | 10 minutes | | 20 minutes | | 30 minutes | |
|---|---|---|---|---|---|---|---|
| | | heating | | heating | | heating | |
| Mass Tag | Measure | Mean | CV | Mean | CV | Mean | CV |
| Ce140 | Max Pixel | 108.3 | 2227.2 | 86.3 | 2415.9 | 31.1 | 1738.7 |
| Intensity | | | | | | | |
| | Integral | 10.1% | 5.2% | 10.5% | 3.9% | 11.9% | 4.8% |
| Eu151 | Max Pixel | 97.8 | 1873.2 | 76.6 | 2116.0 | 27.5 | 1542.4 |
| Intensity | | | | | | | |
| | Integral | 10.9% | 4.7% | 13.8% | 3.2% | 16.9% | 6.1% |
| Eu153 | Max Pixel | 120.2 | 2339.3 | 91.0 | 2633.6 | 33.3 | 1928.2 |
| Intensity | | | | | | | |
| | Integral | 9.6% | 4.6% | 10.6% | 3.7% | 14.1% | 6.5% |
| Ho165 | Max Pixel | 107.1 | 2013.3 | 80.3 | 2230.0 | 29.2 | 1659.5 |
| Intensity | | | | | | | |
| | Integral | 9.9% | 4.8% | 11.4% | 3.8% | 17.7% | 6.9% |
| Lu175 | Max Pixel | 114.1 | 2260.2 | 87.7 | 2493.0 | 31.0 | 1824.9 |
| Intensity | | | | | | | |
| | Integral | 9.8% | 4.3% | 13.7% | 4.4% | 14.7% | 5.5% |

[symbol]

indicates data missing or illegible when filed

Comparison of the effect of heating on the bead diameter, the average integral signal intensity per fused bead (for the Lu175 mass channel), and average laser energy required to completely ablate a bead, is given in Table 4.

TABLE 4
Effect of heating duration on bead diameter, average
integral signal intensity per fused bead, and average
laser energy required to completely ablate a bead.

| | 10 min. | 20 min. | 30 min. |
|---|---|---|---|
| | heating | heating | heating |
| Approx. Bead Diameter (μm) | 5.3 | 8.4 | 12.4 |
| Average Max. Pixel Intensity | 114.1 | 87.7 | 31.0 |
| (Lu175) | | | |
| Laser Energy Required to | 7 | 5 | 1 |
| Completely Clear Bead (dB) | | | |

1. An imaging mass calibrator comprising a sample carrier with at least one reference particle fused to the sample carrier, and wherein the at least one reference particle comprises at least one reference atom.

2. The imaging mass calibrator of claim 1, wherein the sample carrier comprises at least 2, such as at least 3, at least 5, at least 10, at least 50, at least 100, at least 500, at least 1000, at least 2000, at least 5,000, or at least 10,000 fused reference particles.

3. The imaging mass calibrator of any preceding claim, comprising more than one set of fused reference particles, for example wherein the sample carrier comprises at least two, at least three, at least four, at least five, at least six, at least seven, at least eight, or at least ten sets of at least one fused reference particle.

4. The imaging mass calibrator of claim 3, wherein each set of at least one fused reference particle comprises a different reference atom of different atomic mass.

5. The imaging mass calibrator of claim 3, wherein each set of at least one fused reference particle comprises a different amount of the same reference atom.

6. The imaging mass calibrator of claim 1, wherein the at least one fused reference particle comprises more than one reference atom of different atomic mass, for example wherein the at least one reference particle comprises at least two, at least three, at least four, at least five, at least six, at least seven, at least eight, at least nine, or at least ten, different reference atoms of different atomic mass.

7. The imaging mass calibrator of claim 6, wherein each reference atom of different atomic mass is present at a different amount.

8. The imaging mass calibrator of claim 6 or 7, comprising more than one set of at least one fused reference particle, for example at least two, at least three, at least four, at least five, at least six, at least seven, at least eight, or at least ten sets of at least one fused reference particle; wherein each set of at least one fused reference particle comprises a different amount of each of the more than one reference atoms of different atomic mass.

9. The imaging mass calibrator of any preceding claim, wherein the fused reference particles comprise n×10⁻⁵-n×10⁵ of each type of reference atom, such as n×10⁻⁴-n×10⁴ of each type of reference atom, n×10⁻³-n×10³ of each type of reference atom, n×10⁻²-n×10² of each type of reference atom, or n×10⁻¹-n×10¹ of each type of reference atom; where n=10,000,000-30,000,000.

10. The imaging mass calibrator of any preceding claim, wherein the at least one reference particle comprises between 1,000-300,000,000, 2,000-200,000,000, 5,000-175,000,000, 50,000-150,000,000, 100,000-125,000,000, 200,000-110,000,000, 1,000,000-100,000,000, 10,000,000-95,000,000, 30,000,000-90,000,000, 40,000,000-80,000,000, or 50,000,000-70,000,000 reference atoms in total.

11. The imaging mass calibrator of any preceding claim, wherein the at least one fused reference particle comprises between 1,000-100,000,000, 5,000-50,000,000, 50,000-40,000,000, 100,000-30,000,000, 200,000-20,000,000, 1,000,000-20,000,000, 10,000,000-20,000,000 or 12,000,000-18,000,000 of each type of reference atom.

12. The imaging mass calibrator of claims 3 to 11, wherein the sample carrier comprises at least two discrete areas, such as at least 3, at least 4, at least 5, at least 6, at least 8, at least 10 discrete areas, wherein each discrete areas comprises a different set of at least one fused reference particle.

13. The imaging mass calibrator of any preceding claim, wherein under continuous operation the variation of the average integral signal intensity per fused reference particle is less than 15% over 24 hours, less than 12%, less than 10%, less than 8%, less than 5% over 24 hours.

14. The imaging mass calibrator of any preceding claim, wherein the fused reference particle has a glass transition temperature of at least 80° C., such as at least 100° C., at least 120° C., at least 140° C., at least 160° C., at least 180° C., or at least 200° C.

15. The imaging mass calibrator of any preceding claim, wherein the fused reference particle comprises a polyester, a polyether, a polyamide, a polyurethane, a polyaniline, a polyolefin, a polyimide, a polysiloxane, a polycarbonate, a polymethacrylate, a polyacrylate, a polymethacrylamide, also including but not limited to poly(cyclopentadiene), poly(vinylidene fluoride), nylon, poly(tetrafluoroethylene), poly(dimethylsiloxane), poly(methylmethacrylate), polyethylene terephthalate, polystyrene, poly(vinylpyridine), combinations thereof and the like.

16. The imaging mass calibrator of any preceding claim, wherein the fused reference particle comprises polystyrene.

17. The imaging mass calibrator of any preceding claim, wherein the fused reference particle comprises a bead.

18. The imaging mass calibrator of any preceding claim, wherein the at least one fused reference particle is a metal-doped bead, for example wherein the fused reference particle is a metal-doped polymer bead, optionally wherein the fused reference particle is a metal-doped polystyrene bead, optionally wherein the beads are EQ4 beads or DM7 beads.

19. The imaging mass calibrator claim 18, wherein the metal doped polymer bead is produced by dispersion or emulsion polymerisation.

20. The imaging mass calibrator of claims 1 to 17, wherein the at least one fused reference particle is a polymer-coated metal nanoparticle.

21. The imaging mass calibrator of claims 1 to 17, wherein the at least one fused reference particle comprises a polymer and the at least one reference atom is covalently attached to the backbone of the polymer.

22. The imaging mass calibrator of claims 1 to 17, wherein the at least one fused reference particle comprises a polymer comprising metal-chelating moieties.

23. The imaging mass calibrator of claim 22, wherein the metal chelating moiety is:
a. a polymer having a degree of polymerization of between approximately 1 and 10,000, such as 5-100, 10-250, 250-5,000, 500-2,500, or 500-1,000; and/or
b. a polymer comprising between approximately 1 and 10,000, such as 5-100, 10-250, 250-5,000, 500-2,500, or 500-1,000 metal-chelating groups, such as wherein each metal chelating group comprises at least four acetic acid groups, for example wherein the metal chelating groups are 1,4,7,10-tetraazacycloidodecane-1,4,7,10-tetraacetic acid (DOTA), diethylene triamine pentaacetic acid (DTPA), or combinations thereof, optionally wherein each metal chelating group is attached to a polymer subunit derived from either a substituted polyacrylate, polyacrylamide, polymethacrylate, or polymethacrylamide,
c. for example wherein the method further comprises the step of loading at least one metal reference atom onto the polymer, to produce a reference particle comprising a polymer comprising between approximately 1 and 10,000, such as 5-100, 10-250, 250-5,000, 500-2,500, or 500-1,000 chelated metal reference atoms.

24. The imaging mass calibrator of claims 22 to 23, wherein the polymer is polystyrene further comprising metal-chelating groups, for example, wherein the polymer is a polystyrene-polyacrylate copolymer, a polystyrene-polyacrylamide copolymer, a polystyrene-polymethacrylate copolymer, or a polystyrene-polymethacrylamide copolymer; wherein each metal chelating group is attached to a polymer subunit derived from the polyacrylamide, polymethacrylate, or polymethacrylamide.

25. The imaging mass calibrator of claims 21 to 24, wherein the metal-chelating moiety forms part of a surface on a particle.

26. The imaging mass calibrator of any preceding claim, wherein the sample carrier further comprises a sample.

27. The imaging mass calibrator of any preceding claim, wherein the at least one fused reference particle comprises a fluorescence tag.

28. The imaging mass calibrator of claim 27, wherein the fluorescent tag identifies the fused reference particle.

29. The imaging mass calibrator of any preceding claim, wherein the at least one reference atom has an atomic mass in the range of 80-250.

30. The imaging mass calibrator of any preceding claim, wherein the at least one reference atom is selected from the group consisting of lanthanum, cerium, praseodymium, neodymium, promethium, samarium, europium, gadolinium, terbium, dysprosium, holmium, erbium, thulium, ytterbium, lutetium.

31. The imaging mass calibrator claims 3 to 30, wherein each set of at least one reference particle comprises a different coding atom.

32. The imaging mass calibrator of any preceding claim, wherein the sample carrier substrate is selected from inorganic and organic materials, metals, noble metals, metal oxides, mica, silica, ceramics, glass, for example aluminium, cellulose, chitosan, Indium Tin Oxide (ITO), Aluminium oxide (Al2O3), Magnetite (Fe₃O₄), CuOx, Hematite (c-Fe₂O₃), Manganese spiral Ferrite (MnFe₂O₄), Magnesium hydroxide (Mg(OH)₂), Zinc oxide (ZnO), zirconium phosphonate, halloysite, montmorillonite, steel, sapphire, Cadmium selenide (CdSe), Cadmium sulphide (CdS), Gallium Arsenide (GaAs), mica, carbon black, diamond, single walled carbon nanotubes, multiwalled carbon nanotubes, or graphene.

33. The imaging mass calibrator of any preceding claim, wherein the sample carrier comprises a slide, such as a planar microscope slide.

34. The imaging mass calibrator of any preceding claim, wherein the fused reference particles have a diameter of at least 3 μm, at least 5 μm, at least 8 μm, or at least 10 μm.

35. The imaging mass calibrator of any preceding claim, wherein the fused reference particles have a diameter of less than 30 μm, less than 20 μm, less than 15 μm, or less than 10 μm.

36. A method for making an imaging mass cytometry calibrator, comprising the steps of
a. contacting a sample carrier with a suspension comprising at least one reference particle, wherein the at least one reference particle comprises at least one reference atom; and
b. fusing the at least one reference particle onto the sample carrier.

37. The method of claim 36, wherein step a) further comprises drying the sample.

38. The method of claims 36 to 37, wherein the at least one reference particle is suspended in water or ethanol, other alcohols and solvents, mixtures thereof and the like, optionally methanol, propanol, butanol, acetic acid, or acetone.

39. The method of claims 36 to 37, wherein the suspension comprises more than one set of reference particles, for example wherein the suspension comprises at least two, at least three, at least four, at least five, at least six, at least seven, at least eight, or at least ten sets of at least reference particle

40. The method of claim 39, wherein each set of at least one reference particle comprises a different reference atom of different atomic mass.

41. The method of claim 39, wherein each set of at least one reference particle comprises a different amount of the same reference atom.

42. The method of claims 36 to 39, wherein the at least one reference particle comprises more than one reference atom of different atomic mass, for example wherein the at least one reference particle comprises at least two, at least three, at least four, at least five, at least six, at least seven, at least eight, or at least ten different reference atoms of different atomic mass.

43. The method of claim 42, wherein the suspension comprises more than one set of at least one fused reference particle, for example at least two, at least three, at least four, at least five, at least six, at least seven, at least eight, or at least ten sets of at least one fused reference particle; wherein each set of at least one fused reference particle comprises a different amount of each of the more than one reference atoms of different atomic mass.

44. The method of claim 43, wherein each set of at least one reference atom comprises a different amount of each of the more than one different reference atom of different atomic mass.

45. The method of claims 36 to 44, wherein step a) further comprises contacting discrete areas of the sample carrier with at least one additional suspension, for example wherein step a) further comprises contacting discrete areas of the sample carrier with at least two, at least three, at least four, at least five, at least six, at least seven, at least eight, or at least ten, additional suspensions.

46. The method of claim 45, wherein each additional suspension comprises a set of at least one reference particle, wherein the different sets of at least one reference particle each comprise a different reference atom.

47. The method of claim 46, wherein each additional suspension comprises a set of at least one reference particle, wherein the different sets of at least one reference particle each comprise a different amount of the same reference atom.

48. The method of claim 46, wherein each suspension comprises a set of at least one reference particle comprising more than one reference atom of different atomic mass, for example wherein the at least one reference particle comprises two, three, four, five, six, seven, or eight different reference atoms of different atomic mass, wherein each set of at least one reference particle comprises a different amount of each of the more than one different reference atom of different atomic mass.

49. The method of claims 36 to 48, wherein an areas of the sample carrier at least 1 mm from the edge of the sample carrier is contacting with the suspension comprising at least one particle, for example at least 2 mm, at least 3 mm, at least 4 mm, at least 10 mm, from the edge of the slide.

50. The method of claims 36 to 49, wherein the step of fusing the at least one reference particle comprises heating the sample carrier.

51. The method of claims 36 to 49, wherein the step of fusing the at least one reference particle with the sample carrier comprises heating the reference particle at a temperature above the glass transition temperature of the reference particle and subsequently cooling the reference particle below the glass transition temperature of the reference particle.

52. The method of claim 51, wherein the step of heating the at least one reference particle comprises heating the sample carrier.

53. The method of claims 36 to 52, wherein the fusing of the at least one reference particle with the sample carrier is by vitrification.

54. The method of claims 36 to 48, wherein the at least one reference particle is crystalline and the reference particle is fused to the sample carrier by heating the sample carrier at a temperature above the melt temperature of the reference particle.

55. The method of claims 36 to 53, wherein the at least one reference particle is a polystyrene bead and the step of fusing the reference particle is performed by heating the sample carrier above the glass transition temperature of polystyrene.

56. The method of claims 36 to 55, wherein the step of fusing the reference particle is performed by heating sample carrier to a temperature of at least 100° C., at least 120° C., at least 140° C., at least 150° C., at least 160° C., at least 180° C., or at least 200° C.

57. The method of claims 36 to 56, wherein the step of fusing the reference particle is performed by heating the sample carrier to a temperature of at least 150° C., or at least 175° C.

58. The method of claims 51 to 57, wherein heating the sample carrier is performed until the particle has increased in diameter by at least 5%, at least 10%, at least 15%, at least 20%, at least 30%, at least 50%, at least 75%, at least 100%, or at least 150% relative to the reference particle before heating.

59. The method of claims 51 to 53, wherein the step of heating the sample carrier is performed at least 10° C. in excess of the T_{g} of the reference particle, such as at least 20° C., at least 30° C., at least 40° C., at least 50° C., at least 60° C., at least 80° C., or at least 100° C. in excess of the T_{g} of the reference particle.

60. The method of claims 36 to 48, wherein the step of fusing the at least one reference particle to the sample carrier comprises exposing the at least one reference particle to a solvent or mixture of solvents to partially solvate or swell the reference particles.

61. The method of claims 36 to 50, wherein the step of fusing the at least one reference particle to the sample carrier is performed by solvent annealing the reference particle with a solvent or mixture of solvents.

62. The method of claims 60 to 61, wherein the solvent or mixture of solvents is in the vapour phase.

63. The method of claims 60 to 62, wherein the solvent or mixture of solvents has a Hildebrand solubility parameter within at least 2 J^{1/2} m^{-3/2}, at least 1 J^{1/2}M^{-3/2}, at least 0.6 J^{1/2} m^{-3/2}, at least 0.4 J^{1/2} m^{-3/2}, at least 0.2 J^{1/2} m^{-3/2}, at least 0.1 J^{1/2} m^{-3/2}, or substantially the same Hildebrand solubility parameter.

64. The method of claims 36 to 63, wherein the method further comprises the step of preparing a sample on the sample carrier comprising:
i. loading a sample onto the sample carrier;
ii. labelling the sample with at least one mass tag, wherein the mass tag comprises one half of a specific binding pair and at least one labelling atom;
iii. washing the sample; and
iv. drying the sample.

65. The method of claim 64, wherein the sample carrier further comprises a sample such that step a) is performed after the sample has been prepared on the sample carrier.

66. The method of claim 64, wherein steps a) and b) are performed before the sample has been prepared on the sample carrier.

67. The method of claims 36 to 66, wherein the sample carrier comprises a slide, such as a planar microscope slide.

68. A method for monitoring the performance of an instrument; comprising:
a. providing an imaging mass cytometry calibrator comprising a sample carrier with at least one reference particle fused thereto, wherein the at least one fused reference particle comprises at least one labelling atom,
b. determining an average integral signal intensity per fused reference particle, and
c. monitoring the average integral signal intensity per fused reference particle by sampling and detecting elemental composition and amount.

69. The method of claim 68, wherein the integral signal intensity is determined by sampling and ionising sample the material from at least one fused reference particle; wherein sampling and ionising comprises laser ablation followed by separate ionisation of sample material, such as in an ICP, to form sample ions.

70. The method of claim 69, wherein the integral signal intensity per fused reference particle is determined by ablating the whole fused reference particle.

71. The method of claims 69 to 70, wherein calculating the average integral signal intensity per reference particle in steps b) and c) comprises ablating the at least one fused reference particle, for example wherein calculating the average integral signal intensity per fused reference particle comprises ablating at least two, at least three, at least four, at least five, at least six, at least seven, at least eight, at least ten, or at least twenty fused reference particles; and calculating an average integral intensity thereof.

72. The method of claims 69 to 71, wherein step c) comprises ablating at least one fused reference particle to obtain an average integral signal intensity per fused reference particle at least every 10 minutes, at least every 30 minutes, at least every 40 minutes, at least every 60 minutes, at least every 90 minutes, at least every 120 minutes, or at least every 300 minutes.

73. The method of claims 69 to 72, wherein steps b) and c) further comprise using a camera to identify the fused reference particle to be sampled and ionised.

74. The method of claims 69 to 73, wherein the spot size of the laser is smaller than the average longest diameter of the at least one fused reference particle, for example wherein the diameter of the spot size is less than 0.8, such as less than 0.5, less than 0.4, less than 0.3, less than 0.2, or less than 0.1 times the size of the average longest diameter of the bead.

75. The method of claims 69 to 74, wherein the sample ions are detected by a mass spectrometer, for example a quadrupole detector, a magnetic sector detector, a time of flight (TOF) detector, or a tandem mass spectrometer detector.

76. The method of claims 69 to 75, wherein the sample ions are detected by an optical emission spectrometer (OES).

77. The method of claims 68 to 76, wherein the sample carrier further comprises a sample comprising at least one mass tag and step b) is performed before data acquisition for the sample, for example wherein step b) is the initial average integral signal intensity per fused reference particle.

78. The method of claims 68 to 77, wherein the average integral signal intensity per fused reference particle is monitored throughout the imaging of the sample.

79. The method of claims 68 to 78, wherein reference particles are sampled at least two times during imaging of a sample, at least three times, at least four times, at least five times, at least ten times, or more than ten times during imaging of a sample.

80. The method of claims 68 to 79, wherein the imaging of a sample is performed for at least 5 hours, such as at least 10 hours, at least, 15 hours, at least 20 hours, or at least 24 hours, at least 48 hours, at least 72 hours, or at least 96 hours.

81. The method of claims 68 to 80, wherein an average integral signal intensity per fused reference particle is monitored for more than a day, for example, at least two days, at least three days, at least four days, at least five days, or at least one week.

82. The method of claims 68 to 81, wherein a detection of a variation in average integral signal intensity per fused reference particle indicates a flux in instrument sensitivity; for example wherein detection of a variation in average pixel intensity per fused reference particle of greater than 50%, greater than 40%, greater than 30%, greater than 20%, greater than 15%, or greater than 10%, indicates a flux in instrument sensitivity.

83. The method of claim 82, wherein the variation in average integral signal intensity per fused reference particle is measured
i. relatively, as a percentage of the initial average integral signal intensity per fuse reference particle; or
ii. absolutely, comprising comparison of the average integral signal intensity per fused reference particle to a calibration curve.

84. The method of claim 81, wherein the flux in instrument sensitivity indicate flux of the laser or the detector.

85. The method of claims 68 to 84, wherein the method further comprises normalising the signal intensity, comprising the steps of
d. calculating a ratio of the average pixel intensity per fused reference particle determined in step c) to the average pixel intensity per fused reference particle determined in step b), and
e. multiplying the ratio calculated in step d) by the detector intensity detected.

86. The method of claim 85, wherein normalisation of the signal intensity is performed
i. during the imaging of a single sample; and/or
ii. between imaging different samples

87. The method of claims 85 to 86, wherein the signal intensity is normalised for detection of a mass channel; for example wherein the detector intensity is normalised for the detection of a lanthanide, such as Cerium, Europium, Holmium, and/or Lutetium.

88. The method of claim 87, wherein the signal intensity is normalised for the mass channel closest in atomic mass to the at least one labelling atom in the mass tag being sampled and ionised from the sample, for example wherein the signal intensity is normalised for the same mass channel as the at least one labelling atom in the mass tag being sampled and ionised from the sample, or wherein the detector intensity is normalised for a mass channel less than 10 atomic units, within 20 atomic units, within 30 atomic units, or within 40 atomic units away from the atomic mass of the at least one labelling atom in the mass tag being sampled and ionised from the sample.

89. The method of claim 87, wherein the detector intensity is normalised for the mass channel closest in intensity to the at least one labelling atom being sampled and ionised from the sample.

90. Use of at least one reference particle in a method of making an imaging mass calibrator, comprising the steps of:
a. Providing at least one reference particle wherein the at least one reference particle comprises at least one reference atom,
b. Contacting the at least one reference particle with a sample carrier,
c. Fusing the at least one reference particle to the sample carrier.

91. The use of claim 90, wherein the at least one reference particle is fused to the sample carrier by vitrification.

92. The use of claim 90, wherein the at least one reference particle is fused to the sample carrier by solvent annealing.

93. The use of claims 90 to 92, wherein the at least one reference particle comprises n×10⁻⁵-n×10⁵ of each type of reference atom, such as n×10⁻⁴-n×10⁵ of each type of reference atom, n×10⁻³-n×10³ of each type of reference atom, n×10⁻²-n×10² of each type of reference atom, or n×10⁻¹-n×10¹ of each type of reference atom; where n=10,000,000-30,000,000.

94. At least one reference particle for use in a method of making an imaging mass calibrator, wherein the method comprises the steps of:
a. Providing at least one reference particle wherein the at least one reference particle comprises at least one reference atom,
b. Contacting the at least one reference particle with a sample carrier,
c. Fusing the at least one reference particle to the sample carrier.

95. The at least one reference particle of claim 94, wherein the at least one reference particle is fused to the sample carrier by vitrification.

96. The at least one reference particle of claim 94, wherein the at least one reference particle is fused to the sample carrier by solvent annealing.

97. The at least one reference particle of claims 94 to 96, wherein the at least one reference particle comprises n×10⁻⁵-n×10⁵ of each type of reference atom, such as n×10⁻⁴-n×10⁴ of each type of reference atom, n×10⁻³-n×10³ of each type of reference atom, n×10⁻²-n×10² of each type of reference atom, or n×10⁻¹-n×10¹ of each type of reference atom; where n=10,000,000-30,000,000.

98. A suspension of at least one reference particle in a solvent, wherein the at least one reference particle comprising at least one reference atom, wherein the at least one reference particle is capable of being fused to a sample carrier.

99. The suspension of claim 98, wherein the at least one reference particle is capable of being fused to the sample carrier by vitrification.

100. The suspension of claim 98, wherein the at least one reference particle is capable of being fused to the sample carrier by solvent annealing.

101. The suspension of claims 98 to 100, wherein the reference particles are present in a concentration between 1×10⁶ to 1×10¹⁵ particles per ml, for example from 1×10⁷ to 1×10¹³ particles per ml, 1×10⁸ to 1×10¹³ particles per ml, 1×10⁹ to 1×10¹² particles per ml, 1×10⁹ to 1×10¹¹ particles per ml, or about 1×10¹⁰ particles per ml.

102. The suspension of claims 98 to 100, wherein the reference particles are present in a concentration between 1×10⁶ to 1×10¹⁵ particles per ml, for example from 1×10⁷ to 1×10¹³ particles per ml, 1×10⁷ to 1×10¹² particles per ml, 1×10⁷ to 1×10¹⁰ particles per ml, 1×10⁷ to 1×10⁹ particles per ml, or about 1×10⁸ particles per ml.

103. The suspension of claims 98 to 102, wherein each reference particle comprises more than one different reference atom, such as at least two, at least three, at least four, at least five, at least six, at least seven, at least eight, or at least ten different types of reference atom.

104. The suspension of claims 98 to 103, comprising more than one set of reference particles for example at least two, at least three, at least four, at least five, at least six, at least seven, at least eight, or at least ten, sets of reference particles.

105. The suspension of claim 104, wherein each set of at least one reference particle comprises a different concentration of each type of reference atom.

106. A calibration series comprising the suspensions of claims 104 to 105.

107. The calibration series of claim 106, comprising at least one suspension of claims 98 to 103, such as at least two, at least three, at least four, at least five, at least six, at least seven, at least eight, or at least ten, suspension of claims 98 to 103.

108. The calibration series of claim 107, wherein each suspension comprises a set of reference atoms comprising a different number of each type of reference atom.

109. The calibration series of claim 108, comprising suspensions comprising sets of reference particles having between n×10⁻⁵-n×10⁻⁴ of each type of reference atom, n×10⁻⁴ n×10⁻³ of each type of reference atom, n×10⁻³-n×10⁻² of each type of reference atom, n×10⁻²-n×10⁻¹ of each type of reference atom, n×10⁻¹-n×10¹ of each type of reference atom, n×10¹-n×10² of each type of reference atom, n×10²-n×10³ of each type of reference atom, n×10³-n×10⁴ of each type of reference atom, and/or n×10⁴-n×10⁵ of each type of reference atom; where n=10,000,000-30,000,000.

110. Use of the calibration series of claims 106 to 109 in a method of making an imaging mass calibrator, the method comprising the steps of:
a. contacting a sample carrier with the calibration series of claims 106 to 109,
b. fusing the reference particles to the sample carrier.

111. A kit for preparing an imaging mass calibrator comprising at least one reference particle comprising at least one reference atom, wherein the at least one reference particle is capable of being fused to a sample carrier.

112. The kit of claim 111, wherein the at least one reference particle is suspended in a solvent.

113. The kit of comprising the suspensions of claims 104 to 105.

114. The kit comprising the calibration series of claims 106 to 109, wherein each suspension in the calibration series is separate from the other suspensions.

115. The kit of claims 111 to 114 further comprising instructions to fuse the at least one particle to a sample carrier.

116. A method for monitoring the performance of an instrument; comprising:
a. providing a first imaging mass cytometry calibrator comprising a sample carrier comprising a first sample and at least one fused reference particle,
b. samples at least one fused reference particle on the first imaging mass calibrator
c. determining an average integral signal intensity per fused particle for the at least one fused particle,
d. providing at least one additional imaging mass cytometry calibrator comprising a sample carrier comprising a second sample and the same at least one fused reference particle,
e. sampling at least one fused reference particle on the second imaging mass calibrator
f. determining an average integral signal intensity per fused particle for the at least one fused particle
g. comparing the absolute intensities of the average integral signal intensities detected in steps b) and d)
h. normalising the signal intensity

117. The method of claim 116, wherein between steps c) and d) the method further comprises the step of imaging the first sample and between steps f) and g) the method further comprises the step of imaging the second sample using imaging mass cytometry.

118. The method of claim 116, wherein the steps of sampling the at least one fused reference particle and imaging the sample are repeated until the whole sample is imaged.

119. A method for calibrating an imaging mass cytometer comprising the steps of:
a. providing an imaging mass cytometry calibrator comprising a sample carrier with at least one reference particle fused thereto, wherein the at least one reference particle comprises at least one labelling atom;
b. sampling at least one fused reference particle; and
c. determining an average integral signal intensity per fused reference particle.

120. The method of 119, wherein the average integral signal intensity is compared and standardised relative to an expected average integral signal intensity for the at least one fused reference particle.

121. The method of claim 120, wherein the at least one fused reference particle comprises more than one different reference atom, such as at least two, at least three, at least four, at least five, at least six, at least seven, at least eight, or at least ten different types of reference atom.

122. The method of claim 121, wherein the sample carrier comprises more than one fused reference particle, for example wherein the sample carrier comprises two, three, four, five, six, seven, or eight sets of at least one fused reference particle; wherein each set of at least one fused reference particle comprises a different number of each type of reference atom.

123. The method of claim 122, further comprising the steps of:
d. sampling at least one fused reference particle from each set of at least one fused reference particle;
e. determining an average integral signal intensity per fused reference particle for each set;
f. plotting a calibration curve.

124. The method of claims 121 to 123, wherein the different sets of at least one reference particle are located on discrete areas on the sample carrier, for example at least two, at least three, at least four, at least five, at least six, at least seven or at least eight discrete areas.

125. The method of claims 119 to 124, wherein the average pixel intensity per fused reference particle is determined by sampling and ionising the material of at least one fused reference particles; wherein sampling and ionising comprises laser ablation followed by separate ionisation of ablated material, such as in an ICP, to form sample ions.

126. The method of claim 125, wherein the sample ions are detected by a mass spectrometer, for example a quadrupole detector, a magnetic sector detector, a time of flight (TOF) detector, or a tandem mass spectrometer detector.

127. The method of claim 125, wherein the sample ions are detected by an optical emission spectrometer (OES).

128. The method of claims 122 to 127, wherein each set of reference particles comprises a different fluorescent tag; the method further comprising the steps of illuminating the fluorescent tag to cause it to fluoresce and identify the specific set of at least one reference particle on the basis of its fluorescence.

129. The method of claims 122 to 127, where at the at least one reference particle comprises at least one coding atom, wherein the coding atom identifies the reference particle, wherein the method further comprises the step of detecting the coding atom and identifying the set of at least one reference particle.

130. A method of imaging a sample comprising the steps of
a. providing an imaging mass cytometry calibrator, comprising a sample carrier with at least one reference particle fused thereto, wherein the at least one fused reference particle comprises at least one reference atom, and wherein the sample is on the sample carrier;
b. contacting the sample with a solution comprising at least one mass tag, wherein the at least one mass tag comprises at least one labelling atom
c. washing the sample
d. drying the sample
e. sampling at least one fused reference particle
f. determining an average integral signal intensity per fused reference particle
g. performing imaging mass cytometry on the sample to obtain an image.

131. The method of claim 130, wherein the step of performing imaging mass cytometry on the sample comprises sampling the sample to determine the level of the one or more labelling atoms, wherein the level of the one or more labelling atoms corresponds to the copy number of the one or more analytes in the sample.

132. A method of imaging a sample comprising the steps of
a. providing a sample on a sample carrier;
b. preparing an imaging mass cytometry calibrator, wherein the imaging mass cytometer calibrator comprises the sample on the sample carrier, wherein the sample carrier has at least one reference particle fused thereto, wherein the at least one fused reference particle comprises at least one reference atom;
c. contacting the sample with a solution comprising at least one mass tag, wherein the at least one mass tag comprises at least one labelling atom
d. washing the sample
e. drying the sample
f. sampling at least one fused reference particle
g. determining an average integral signal intensity per fused reference particle
h. performing imaging mass cytometry on the sample to obtain an image.

133. The method of claim 132, wherein the step of performing imaging mass cytometry on the sample comprises sampling the sample to determine the level of the one or more labelling atoms, wherein the level of the one or more labelling atoms corresponds to the copy number of the one or more analytes in the sample.

134. Use of the calibrator of claims 1 to 34 in a method of calibrating a mass cytometer, comprising the steps of:
a. providing the imaging mass cytometry calibrator of claims 1 to 34,
b. sampling at least one fused reference particle,
c. determining an average integral signal intensity per fused reference particle.

135. Use of the imaging mass cytometry calibrator of claims 1 to 34 in a method of imaging a sample, comprising the steps of:
a. providing the imaging mass cytometry calibrator of claims 1 to 34, wherein the calibrator further comprises a sample comprising at least one mass tag,
b. performing imaging mass cytometry on the sample to obtain an image.
